(19) United States
(12) Patent Application Publication
(10) Pub. No.: US 2007/0003514 A1
(43) Pub. Date: Jan. 4, 2007

(54) Mono-and bi-functional antibody conjugates as effective adjuvants of protein vaccination

(75) Inventors: Manuel L. Penichet, Los Angeles, CA (US); Gustavo F. Helguera, Los Angeles, CA (US); Sherie L. Morrison, Los Angeles, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 11/193,982

(22) Filed: Jul. 29, 2005

Related U.S. Application Data
(63) Continuation-in-part of application No. 10/118,473, filed on Apr. 5, 2002.
(63) Continuation-in-part of application No. PCT/US03/09136, filed on Mar. 21, 2003.
(60) Provisional application No. 60/692,059, filed on Jun. 16, 2005.

(51) Int. Cl. A61K 39/395; A61K 38/20; A61K 38/19; C07K 16/46; C07K 14/54; C07K 14/52

(52) U.S. Cl. 424/85.1; 424/85.2; 530/351; 530/391.1; 424/178.1

(57) ABSTRACT

The present invention provides methods of use of various antibody-immunostimulant fusion proteins as adjuvants of antigenic protein vaccinations to elicit humoral and/or cellular immune responses in vaccinated subjects. Compositions which include these fusion proteins and innate and/or exogenous antigenic proteins are also provided.

### STATEMENT AS TO RIGHTS TO INVENTIONS MADE UNDER FEDERALLY SPONSORED RESEARCH AND DEVELOPMENT

This invention was made with Government support under Grant Nos. CA86915, CA087990, CA107023, AI139187 and AI29470, awarded by the National Institutes of Health and Army. The Government of the United States of America has certain rights in this invention.

### CROSS-REFERENCE TO RELATED APPLICATIONS

[Not Applicable]

### FIELD OF THE INVENTION

This invention pertains to the field of immunology and vaccine development. In particular, this invention provides novel immunostimulatory agents that can direct an immune response to particular (e.g., disease-related) antigens.

### BACKGROUND OF THE INVENTION

Despite considerable advancement in the therapy of various tumors and cancers, residual disease is still a major problem in the clinical management of these conditions. Additionally, treatment and especially prevention of infectious diseases remains a continuing concern due to, e.g., spread of viral diseases such as HIV and emergence of treatment-resistant variants of more well known diseases such as tuberculosis, staphylococcus infection, etc.

In the case of tumor treatment, chemotherapeutic strategies are necessarily limited by severe toxicities, and are of limited efficacy against non-proliferating tumor cells. Therefore, new methods emphasizing non-chemotherapeutic approaches are desired. For example, treatment of patients with advanced HER2/neu expressing tumors (e.g., breast cancers) through use of a humanized anti-HER2/neu monoclonal antibody, Trastuzumab (previously known as rhuMAb HER2), directed at the extracellular domain of HER2/neu can lead to a measurable response in some patients with tumors that overexpress the HER2/neu oncoprotein. However, only a subset of patients treated with Trastuzumab show an objective response, and although a combination of Trastuzumab with chemotherapy enhances its anti-tumor activity, still not all patients respond positively. Furthermore, even more desirous than an effective treatment for such tumors would be an effective prevention of them (e.g., especially in individuals with a family history of particular cancers).

Previously, antibody-(IL-2) fusion proteins have been the best characterized and most broadly used in successful anti-tumor experiments using animal models (see, e.g., Penichet and Morrison, 2001, “Antibody-cytokine fusion proteins for the therapy of cancer” J Immunol Met 248:91-101). Numerous studies have explored various combinations of antibodies and, e.g., IL-2, as direct targeting agents of tumor cells. For example, a tumor specific antibody-(IL-2) fusion protein was previously developed by the inventors, and comprised a human IgG3 specific for the idiotype (Id) of the Ig expressed on the surface of the B cell lymphoma 38C13 with human IL-2 fused at the end of the C_{H}3 domain. See, Penichet et al., 1998 “An IgG3-IL-2 fusion protein recognizing a murine B cell lymphoma exhibits effective tumor imaging and antitumor activity” J Interferon Cytokine Res 18:597-607. That antibody fusion protein, IgG3-C_{H}3-(IL-2), was expressed in Sp2/0 and was properly assembled and secreted. Anti-Id IgG3-C_{H}3-(IL-2) has a half-life in mice of approximately 8 hours, which is 17-fold longer than the half-life reported for IL-2 (i.e., when not fused to another domain), and it showed a better localization of subcutaneous tumors in mice than the anti-Id IgG3 by itself. Most importantly, the anti-Id IgG3-C_{H}3-(IL-2) showed enhanced anti-tumor activity compared to the combination of antibody and IL-2 administered together. Again, see, Penichet et al., 1998, supra. Additionally, a chimeric anti-Id IgG1-(IL-2) fusion protein (chS5A8-IL-2) expressed in P3X63Ag8.653 has shown more effectiveness in the in vivo eradication of the 38C13 tumor than the combination of the anti-Id antibody and IL-2 or an antibody-(IL-2) fusion protein with an irrelevant specificity. See, Liu et al., 1998 “Treatment of B-cell lymphoma with chimeric IgG and single-chain Fv antibody-interleukin-2 fusion proteins” Blood 92:21030-12.

Another example of previous antibody fusion proteins in cancer treatment involved chimeric anti-GD₂ IgG1-(IL-2) fusion protein (ch14.18-IL-2) produced in Sp2/0 cells. See, Becker et al., 1996 “T cell-mediated eradication of murine metastatic melanoma induced by targeted interleukin 2 therapy” J Exp Med 183:2361-6; Becker et al., 1996 “An antibody-interleukin 2 fusion protein overcomes tumor heterogeneity by induction of a cellular immune response” Proc Natl Acad Sci USA 93:7826-31; and Becker et al., 1996 “Long-lived and transferable tumor immunity in mice after targeted interleukin-2 therapy” J Clin Invest 98:2801-4. The ch14.18-IL-2 treatment of mice which had pulmonary and hepatic metastases, as well as subcutaneous GD₂ expressing B16 melanoma, resulted in a specific and strong anti-tumor activity. This anti-tumor activity was significant compared to antibody (ch14.18) and IL-2 or irrelevant antibody-(IL-2) fusion proteins and resulted in the complete eradication of the tumor in a number of animals. See, Becker references, supra. Similar results have been obtained in mice bearing CT26-KSA hepatic and pulmonary metastases and treated with a humanized anti-KSA antibody-IL-2 fusion protein (huKS1/4-IL-2) produced in NS0. See, Xiang et al., 1997 “Elimination of established murine colon carcinoma metastases by antibody-interleukin 2 fusion protein therapy” Cancer Res 57:4948-55 and Xiang et al., 1999 “T cell memory against colon carcinoma is long-lived in the absence of antigen” J Immunol 163:3676-83.

Other examples of antibody fusion molecules include a chimeric anti-human MHC class II IgG1 fused to GMCSF (chCLL-1/GMCSF) expressed in NS0 (see, Hornick et al., 1997 “Chimeric CLL-1 antibody fusion proteins containing granulocyte-macrophage colony-stimulating factor or interleukin-2 with specificity for B-cell malignancies exhibit enhanced effector functions while retaining tumor targeting properties” Blood 89:4437-47) and a humanized anti-HER2/neu IgG3 fused to IL-12 (see, Peng et al., 1999, “A single-chain IL-12 IgG3 antibody fusion protein retains antibody specificity and IL-12 bioactivity and demonstrates antitumor activity” J Immunol 163:250-8), IL-2 (see, Penichet et al., 2001, “A recombinant IgG3-(IL-2) fusion protein for the treatment of human HER2/neu expressing tumors” Human Antibodies 10:43-49) and GMCSF expressed in P3X63Ag8.653 (see, Dela Cruz et al., 2000, “Recombinant anti-human HER2/neu IgG3-(GMCSF) fusion protein retains antigen specificity, cytokine function and demonstrates anti-tumor activity” J Immunol 165:5112-21).

In all of the above work, it is important to note that the antibody-cytokine fusion proteins containing IL-2, IL-12, or GMCSF, etc. have been used as direct antitumor agents which directly targeted tumors in animal models. The antibody fusion proteins bound to antigens on tumor surfaces, thus increasing the local concentration of, e.g., Il-2, etc. around the tumor. The increased, e.g., IL-2, thus lead to anti-tumor activity in some cases. See, e.g., Penichet, et al. 2001, supra.

Additionally, some prior work by the inventors described linking antigens to IL-2 via an IgG3-(IL-2) fusion protein with affinity for a convenient hapten antigen, dansyl (DNS). See, Harvill et al., 1996 “In vivo properties of an IgG3-Il-2 fusion protein. A general strategy for immune potentiation” J Immunol 147:3165-70. The antigen used in this work was highly artificial (bovine serum albumin) rather than a disease-related antigen. Using hapten-conjugated-bovine serum albumin (DNS-BSA) as a model antigen the inventors showed an antibody response elicited by anti-DNS-IgG3-(IL-2)-bound DNS-BSA injected into mice increased over that of DNS-BSA-Sepharose, anti-DNS-IgG3-bound DNS-BSA, or a non-specific IgG3-(IL-2)-bound DNS-BSA. Although, the binding of the antibody-(IL-2) fusion protein to the antigen (non-covalent physical linkage) was shown to enhance the immune response (see, Harvill et al., 1996, supra), only one antibody fusion protein (antibody-(IL-2) fusion protein was used and the study was restricted to the characterization of the humoral (antibody) immune response. Also, unfortunately, use of the dansyl group may create a low level of stability between the antigens and the antibodies. Such instability could be problematic in proper immune stimulation treatments in vivo. Additionally, the use of dansyl, entails the possibility that the dansyl groups could mask or alter specific epitopes on the antigen it is linked to, thus, interfering with proper immune response stimulation in subjects.

In the case of infectious diseases, numerous bacteria (such as Staphylococcus aureus), viruses, mycoplasms, fungi, parasites, etc. present a serious problem. For example, the bacteria Staphylococcus aureus is a common cause of hospital-acquired infections that result in high mortality. Staphylococcus aureus can cause, e.g., pneumonia, endocarditis, osteomyelitis, septic arthritis, postoperative wound infections, septicemia, toxic shock, etc. Unfortunately, many bacterium, including many strains of Staphylococcus aureus, are resistant to first-line drugs such as synthetic penicillins (e.g., methicillin). Other bacteria, including some strains of Staphylococcus aureus, are resistant to multiple drugs, including the so-called antibiotic of last resort, vancomycin. In the case of other infectious agents (e.g., viruses, fungi, etc.) no effective drug treatment may exist. See, e.g., Nickerson et al., 1995 “Mastitis in dairy heifers: initial studies on prevalence and control” J Dairy Sci 78:1607-18; Lowy, 1998 “Staphylococcus aureus infections” N Engl J Med 339:520-32; McKenney et al., 1999 “Broadly protective vaccine for Staphylococcus aureus based on an in vivo-expressed antigen” Science 284:1523-7; and Lorenz et al., 2000 “Human antibody response during sepsis against targets expressed by methicillin resistant Staphylococcus aureus” FEMS Immunol Med Microbiol 29:145-53. The existence of multiple drug resistant strains of bacterium (and, indeed, of other infectious agents such as fungi, mycoplasms, etc.) raises the specter of untreatable infections and presents an ongoing challenge to the medical and public health communities. Much previous work has been done on generation of vaccines (e.g., both DNA and protein vaccines) for numerous infectious organisms (especially viruses) and such work is well known to those skilled in the art.

A welcome addition to the art would be a convenient method of therapeutic and/or prophylactic treatment to potentiate an effective immune response (humoral and/or cellular) against antigens of tumors and infectious diseases. The current invention provides these and other approaches and methods in treatment.

### SUMMARY OF THE INVENTION

The present invention provides methods of use of various antibody-immunostimulant chimeric moieties (e.g., protein fusions) as adjuvants for antigenic protein vaccinations and methods of prophylactically and/or therapeutically treating a disease state in a subject. Compositions comprising the chimeric moieties and antigens of the invention are also provided.

In one aspect, the invention comprises a composition comprising an an antibody-immunostimulant chimera (chimeric moiety) where the chimera is capable of acting as an effective adjuvant of a disease related antigen. In some embodiments of this aspect, the composition also includes the disease related antigen. In certain embodiments the antibody-immunostimulant chimera has antibody specificity against the disease related antigen.

Certain preferred chimeric moieties comprise an antibody directed against an antigen (e.g. a disease-producing antigen) attached (directly or thorugh one or more linkers) to one or more immunostimulatory molecules (e.g., to two immunostimulatory molecules, three immunostimulatory molecules, four or more immunostimulatory molecules). In certain embodiments, where two or more immunostimulatory molecules are present, the immunostimulatory molecules are different species. Certain embodiments also contemplate the use of two or more antibodies in conjunction, with one, two, or more immunostimulatory molecules. In certain embodiments the chimeric moiety is a fusion protein where the antibody is coupled directly or thorugh a peptide linker to one or more immunostimulants (immunostimulatory molecules). The immunostimulant domain(s) of the chimeric moieties (e.g., fusion proteins) in these compositions can, in certain embodiments comprise a cytokine (or a sequence or subsequence thereof), a chemokine (or a sequence or subsequence thereof), or an immunostimulant other than a chemokine or cytokine. Examples of such immunostimulant domains include, but are not limited to cytokines, chemokines, interleukins, interferons, C-X-C chemokines, C-C family chemokines, C chemokines, CX3C chemokines, super antigens, growth factors, IL-1, IL-2, IL-4, IL-6, IL-7, IL-8, IL-10, IL-12, IL-13, IL-17, IL-18, RANTES, mip1α, mip1β, GMCSF, GCSF, gamma interferon, alpha interferon, TNF, CSFs, mip2α, mip2β, PF4, platelet basic protein, hIP10, LD78, Act-2, MCAF, 1309, TCA3, IP-10, lymphotactin, fractalkine, KLH, and fragments thereof of any of the above.

The antibody domain/component of the chimeric moieties in the compositions of the invention optionally includes, but is not limited to, an antibody specific for a HER2/neu antigen, a tumor antigen, a bacterial antigen, a viral antigen, a mycoplasm antigen, a fungal antigen, a prion antigen, an autoimmune disorder antigen, an antigen from a parasite (e.g., an infectious mammalian parasite), and the like. In certain embodiments, such fusion proteins comprise antibody domains specific for antigens other than tumor antigens. In various embodiments, the antibody-immunostimulant chimeric moieties in the compositions of the invention comprise an antibody fragment, or an Fab domain, an Fab′ domain, an F(ab′)₂ domain, an F(ab)₂domain, an scFv domain, IgG, IgA, IgE, IgM, IgD, IgG1, IgG2, or IgG3.

Also, in some embodiments of the compositions of the invention, the antigen comprises, e.g., a soluble antigen, a soluble antigen bound to a matrix, an insoluble antigen bound to a matrix, an insoluble aggregate of antigens, a nonviable cell-associated antigen, or a nonviable organism-associated antigen, or an antigen conjugated with a liposome. Additionally, such antigen can comprise, e.g., HER2/neu (or HER2/neu shed from a tumor cell) or fragments thereof. Additionally, the antigen in such compositions optionally comprises: an antigen other than a tumor antigen, an antigen arising from a subject, an antigen arising from a disease state within the subject, an antigen arising from a disease related organism within a subject (e.g., a disease state caused by one or more of a tumor, a bacteria, a virus, a mycoplasm, a fungus, a prion, an autoimmune disorder, or an infectious parasite such as an infectious parasite of a mammal, etc.). The antigen can also comprise a tumor antigen, a bacterial antigen, a viral antigen, a mycoplasm antigen, a prion antigen, an autoimmune disorder related antigen, or an infectious parasite antigen. In some embodiments herein, the antigen is an exogenous antigen (which is optionally substantially identical to an antigen arising from a subject, or from a disease state within a subject or from a disease related organism within the subject).

In certain embodiments comprising a chimeric moiety as described herein in combination with an antigen, the number of antigen molecules and the number of chimera molecules are approximately 1:1. In certain other embodiments, they are in ratios where the number of antigen molecules is greater than or lesser than the number of chimera molecules, or where the chimeric moieties are substantially saturated by the antigen molecules, or where the antigen molecules are substantially saturated by the chimera molecules.

In various embodiments the compositions of the invention are optionally incubated for a selected period of time and under selected conditions (e.g., overnight at 4° C., etc. or for even brief periods of time such as 1 second or less, etc.). The compositions of the invention can also, optionally comprise an excipient (e.g., a pharmaceutically acceptable excipient).

In certain embodiments the invention comprises a method of administering an immunological composition by providing a composition comprising one or more chimeric moieties, e.g., as described above and administering the composition to a subject where the composition acts as an effective adjuvant to a disease related antigen and where the composition elicits an immune response in a subject. Certain embodiments of this aspect involve the administration of such compositions along with providing a disease related antigen (e.g., administering the chimeric moiety and the antigen to a subject where the composition acts as an effective adjuvant of the antigen). In some embodiments, the chimeric moiety comprises a cytokine (or a sequence or subsequence thereof), a chemokine (or a sequence or subsequence thereof), or an immunostimulant other than a chemokine or cytokine. In other embodiments of this aspect, the method uses fusion proteins comprising an immunostimulant domain such as (but not limited to), e.g., cytokines, chemokines, interleukins, interferons, C-X-C chemokines, C-C family chemokines, C chemokines, CX3C chemokines, super antigens, growth factors, IL-1, IL-2, IL-4, IL-6, IL-7, IL-8, IL-10, IL-12, IL-13, IL-17, IL-18, RANTES, mip1α, mip1β, GMCSF, GCSF, gamma interferon, alpha interferon, TNF, CSFs, mip2α, mip2β, PF4, platelet basic protein, hIP10, LD78, Act-2, MCAF, 1309, TCA3, IP-10, lymphotactin, fractalkine, KLH, and fragments thereof of any of the above.

The antibody domain of the chimeric moieties used in the methods of this invention are, in certain embodiments, specific for, e.g., HER2/neu antigen, a tumor antigen, a bacterial antigen, a viral antigen, a mycoplasm antigen, a fungal antigen, a prion antigen, an autoimmune disorder related antigen, an infectious parasite antigen (e.g., a parasite of a mammal). In certain embodiments the antibody domain is specific for antigen comprising an antigen other than a tumor antigen. In various embodiments the antibody domain of the chimeric moieties are optionally (but are not limited to), e.g., an antibody fragment, an Fab domain, an Fab′ domain, an F(ab′)₂ domain, an F(ab)₂domain, an scFv domain, IgG, IgA, IgE, IgM, IgD, IgG1, IgG2, IgG3, and the like. In certain embodiments of these methods, the chimeric moiety (e.g., fusion protein) has antibody specificity for the antigen.

Various methods herein can also encompass embodiments where the antigen comprises, e.g., a tumor antigen, a bacterial antigen, a viral antigen, a mycoplasm antigen, a prion antigen, an autoimmune disorder related antigen, a parasite antigen (e.g., one infecting a mammal), an antigen other than a tumor antigen, an antigen arising from the subject, an antigen arising form a disease state within the subject, or an antigen from a disease related organism within the subject. In various embodiments the disease state within the subject that gives rise to such antigens, can be caused by, e.g., a tumor, a bacteria, a virus, a mycoplasm, a fungus, a prion, an autoimmune disorder, a parasite (e.g., one infecting a mammal), and the like. The antigens in this aspect of the invention can be, optionally, exogenous antigens, which can, in certain embodiments, be substantially identical to a disease related antigen arising from a subject, arising from a disease state within a subject, or arising from a disease related organism within a subject. Such exogenous antigen is can be administered prior to administration of the antibody-immunostimulant fusion proteins, or after the fusion proteins are administered to the subject, or approximately concurrently with the fusion proteins to the subject. In various embodiments prior to the concurrent administration the antigen and the fusion protein can be incubated for a specific time period and under specific conditions (e.g., from 1 second or almost instantaneous incubation up to overnight or longer; at, e.g., 4° C., etc.). In various embodiments the antigen used in the methods in this aspect of the invention can also optionally comprise, e.g., HER2/neu, HER2/neu shed from tumor cells, or fragments of such HER2/neu.

In certain embodiments, more than one chimeric moiety can be used. Such multiple chimeric moieties (e.g., fusion proteins) can comprise different immunostimulant domains (e.g., such as ones chosen from (but not limited to) non-cytokine/non-chemokine molecules, cytokines, chemokines, interleukins, interferons, C-X-C chemokines, C-C family chemokines, C chemokines, CX3C chemokines, super antigens, growth factors, IL-1, IL-2, IL-4, IL-6, IL-7, IL-8, IL-10, IL-12, IL-13, IL-17, IL-18, RANTES, mip1α, mip1β, GMCSF, GCSF, gamma interferon, alpha interferon, TNF, CSFs, mip2α, mip2β, PF4, platelet basic protein, hIP10, LD78, Act-2, MCAF, 1309, TCA3, IP-10, lymphotactin, fractalkine, KLH, and fragments thereof of any of the above. Furthermore, the multiple chimeric moieties in the methods of this aspect optionally have different specificity. The multiple fusion proteins can be specific for, e.g., different antigens on a single molecule, different antigens on a single cell, different antigens on a single tumor, or different antigens on a single organism (e.g., a virus, bacteria, fungus, mycoplasm, prion, parasite), etc. The methods of administering an immunological composition also include embodiments where such administration elicits an immune response in a subject.

In certain aspects, the current invention also includes methods of prophylactically and/or therapeutically treating a disease state in a subject. Such methods include administering an effective amount of an antibody-immunostimulant fusion protein to the subject, where the chimeric moiety, (e.g., fusion protein) acts as an effective adjuvant of a disease related antigen (e.g., one arising from the subject, arising from a disease state within the subject, or arising from a disease related organism within the subject) and where the administration elicits an immune response within the subject against the disease related antigen (or closely related antigens). Such method of prophylactically and/or therapeutically treating a disease state also optionally includes administering to the subject an effective amount of an antibody-immunostimulant fusion protein and administering a disease related antigen where the chimeric moiety (e.g., fusion protein) comprises an effective adjuvant of the disease related antigen.

Definitions

The term “treat” when used with reference to treating, e.g. a pathology or disease refers to the mitigation and/or elimination of one or more symptoms of that pathology or disease, and/or a reduction in the rate of onset or severity of one or more symptoms of that pathology or disease, and/or the prevention of that pathology or disease.

The terms “polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

A “chimeric molecule” or “chimeric moiety” is a molecule or moiety in which two or more molecules that exist separately in their native state are joined together to form a single molecule or moiety having the desired functionality of all of its constituent molecules. While the chimeric molecule may be prepared by covalently linking two molecules each synthesized separately, one of skill in the art will appreciate that where the chimeric molecule is a fusion protein, the chimera may be prepared de novo as a single “joined” molecule.

The phrase “in conjunction with” when used in reference to the use of chimeric moieties with antigen(s) as described herein indicates that the chimeric moiety and the antigen are administered so that there is at least some chronological overlap in their physiological activity on the organism. Thus the chimeric moiety and the antigen can be administered simultaneously (e.g. together or as a complex) and/or sequentially. In sequential administration there may even be some substantial delay (e.g., minutes or even hours or days) before administration of the second agent as long as the first administered agent has exerted some physiological alteration on the organism when the second administered agent is administered or becomes active in the organism.

The term “subject” as used herein includes, but is not limited to, a mammal, including, e.g., a human, non-human primate (e.g., monkey), mouse, pig, cow, goat, rabbit, rat, guinea pig, hamster, horse, monkey, sheep, or other non-human mammal, a non-mammal, including, e.g., a non-mammalian vertebrate, such as a bird (e.g., a chicken or duck) or a fish; and a non-mammalian invertebrate. In some embodiments, the methods and compositions of the invention are used to treat (both prophylactically and/or therapeutically) non-human animals. Many commercially important animals are susceptible to various cancers and, especially of concern, to various viral/bacterial, etc. infections which are optionally treated with the current invention.

The term “pharmaceutical composition” herein means a composition suitable for pharmaceutical use in a subject, including an animal or human. A pharmaceutical composition generally comprises an effective amount of an active agent (e.g., the antibody-immunostimulant fusion proteins and antigenic protein vaccinations of the invention) and a pharmaceutically acceptable carrier (e.g., a buffer, adjuvant, or the like).

The term “effective amount” means a dosage or amount sufficient to produce a desired result. The desired result may comprise an objective or subjective improvement in the recipient of the dosage or amount (e.g., long-term survival, decrease in number and/or size of tumors, effective prevention of a disease state, etc.).

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as a protein vaccination and its antibody-immunostimulant fusion protein adjuvant, or composition thereof, that, when administered to a subject who does not display signs or symptoms of a pathology, disease or disorder (or who displays only early signs or symptoms of a pathology, disease, or disorder) diminishes, prevents, or decreases the risk of the subject developing the pathology, disease, or disorder. A “prophylactically useful” agent or compound (e.g., a protein vaccination and its antibody-immunostimulant fusion protein adjuvant) refers to an agent or compound that is useful in diminishing, preventing, treating, or decreasing development of a pathology, disease or disorder.

A “therapeutic treatment” is a treatment administered to a subject who displays symptoms or signs of pathology, disease, or disorder, in which treatment is administered to the subject for the purpose of diminishing or eliminating those signs or symptoms of pathology, disease, or disorder. A “therapeutic activity” is an activity of an agent, such as a protein vaccination and its antibody-immunostimulant fusion protein adjuvant, or composition thereof, that eliminates or diminishes signs or symptoms of a pathology, disease or disorder, when administered to a subject suffering from such signs or symptoms. A “therapeutically useful” agent or compound (e.g., a protein vaccination and its antibody-immunostimulant fusion protein adjuvant) indicates that an agent or compound is useful in diminishing, treating, or eliminating such signs or symptoms of the pathology, disease or disorder.

As used herein, an “antibody” refers to a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (V_{H}) refer to these light and heavy chains, respectively.

Antibodies exist as intact immunoglobulins or as a number of well characterized fragments produced by digestion with various peptidases. Thus, for example, pepsin digests an antibody below the disulfide linkages in the hinge region to produce F(ab′)₂, a dimer of Fab which itself is a light chain joined to V_{H}-C_{H}1 by a disulfide bond. The F(ab′)₂ may be reduced under mild conditions to break the disulfide linkage in the hinge region thereby converting the F(ab′)₂dimer into an Fab′ monomer. The Fab′ monomer is essentially an Fab with part of the hinge region (see, Fundamental Immunology, W. E. Paul, ed., Raven Press, N.Y. (1999), for a more detailed description of other antibody fragments). While various antibody fragments are defined in terms of the digestion of an intact antibody, one of skill will appreciate that such Fab′ fragments, etc. may be synthesized de novo either chemically or by utilizing recombinant DNA methodology. Thus, the term antibody, as used herein also includes antibody fragments either produced by the modification of whole antibodies or synthesized de novo using recombinant DNA methodologies. Antibodies include single chain antibodies, including single chain Fv (sFv or scFv) antibodies in which a variable heavy and a variable light chain are joined together (directly or through a peptide linker) to form a continuous polypeptide.

An “immunostimulant,” or “immunostimulatory” molecule or domain or the like, herein refers to a molecule or domain, etc. which acts (or helps to act) to stimulate or elicit an immune response or immune action in a subject (either cellular or humoral or both). Typical examples of such molecules include, but are not limited to, e.g., cytokines and chemokines. Cytokines act to, e.g., stimulate humoral and/or cellular immune responses. Typical examples of such include, e.g., interleukins such as IL-2, IL-12, etc. Chemokines act to, e.g., selectively attract various leukocytes to specific locations within a subject. They can induce both cell migration and cell activation. Common examples of chemokines include, e.g., RANTES, C-X-C family molecules, Il-8, mip1α, mip1β, etc. For further information, see, e.g., Arai, K. et al, 1990, “Cytokines: coordinators of immune and inflammatory responses” Annu Rev Biochem 59:783+; Taub, 1996 “Chemokine-Leukocyte Interactions. The Voodoo That They Do So Well” Cytokine Growth Factor Rev 7:355-76.

A “disease related antigen” refers to an antigenic protein, peptide, carbohydrate, lipid, nucleic acid, or combination of any of such, which arises or is present in a subject due to a disease state (e.g., such as cancer or autoimmune disorders) or due to an infectious organism (e.g., such as from infection of a subject with such organisms or infectious agents as bacteria, viruses, prions, mycoplasms, fungi, parasites, etc.). The disease related antigen is optionally either wholly or partially soluble when used as a protein vaccination, alternatively such antigen is a soluble antigen bound to a matrix (e.g., a latex bead or other bead, etc.), an insoluble antigen bound to a matrix (e.g., a latex or other bead, etc.), an insoluble aggregate of antigens, a nonviable cell-associated antigen, a nonviable organism-associated antigen, or an antigen conjugated with a liposome, etc. In some embodiments the fusion protein optionally targets a dead or dying (e.g., apoptotic) disease-related cell/organism which comprises one or more disease related antigen. In some embodiments herein the disease related antigen is exogenous. In other words, such antigen is from outside a subject. An exogenous disease related antigen is optionally identical or substantially identical to an innate or non-exogenous disease related antigen (e.g., one arising from within a subject, or from a disease state and/or infectious organism within a subject, etc.).

### BRIEF DESCRIPTION OF THE DRAWINGS

FIG. 1: Schematic diagrams of exemplary antibody-immunostimulant fusion proteins utilized by of the invention.

FIG. 2: Schematic diagram showing creation of immunocomplexes of antibody fusion proteins utilized by the invention and soluble antigens and presentation of such complexes to an antigen presenting cell.

FIGS. 3A-3C: Illustration of anti-tumor activity of exemplary fusion proteins/antigenic vaccination treatments of the invention in vaccinated mice challenged with TUBO.

FIG. 4, Panels A-C: Illustration of the influence of sera on the in vitro proliferation of TUBO and SK-BR-3 cells.

FIG. 5: Illustration of murine anti-ECD^{HER2} antibody response in vaccinated mice.

FIG. 6: Panels A-C: Characterization of anti-ECD^{HER2} IgG of transferred immune sera of vaccinated mice.

FIG. 7, Panels A-B: In vitro stimulation of proliferation of splenocytes of vaccinated mice by ECD^{HER2} protein.

FIG. 8, Panels A-B: In vitro IFN-γ production by stimulated splenocytes from vaccinated mice.

FIG. 9: Illustration of murine anti-protein A antibody response in vaccinated mice.

FIG. 10: Illustration of murine anti-protein A (bound on Cowan I) antibody response in vaccinated mice.

FIGS. 11A, 11B, and 11C illustrate the structure, assembly, size and antigen binding activity of antibody fusion proteins. FIG. 11A: Diagram of antibody fusion proteins. The AbFPs IgG3-(IL-2), IgG3-(GM-CSF), (IL-12)-IgG3 were combined to produce the bi-AbFPs anti-HER2/neu (IL-12)-IgG3-(IL-2) and (IL-12)-IgG3-(GM-CSF). FIG. 11B: SDS-PAGE analysis of purified AbFPs specific for HER2/neu. The AbFPs were affinity purified using protein A and analyzed by SDS-PAGE in the absence (top panel) or presence (bottom panel) of reducing conditions. FIG. 11C: AbFPs can bind to the ECD^{HER2}. Microtiter plates were coated with recombinant human ECD^{HER2} and incubated with serial dilutions of either anti-dansyl IgG3 (isotype control), IgG3-(IL-2), IgG3-(GM-CSF), (IL-12)-IgG3, (IL-12)-IgG3-(GM-CSF), and (IL-12)-IgG3-(IL-2) followed by goat anti-human IgG AP-conjugate. Measurements were made simultaneously and top panel shows data and AbFP controls for (IL-12)-IgG3-(IL-2); while bottom panel shows the data corresponding to (IL-12)-IgG3-(GM-CSF) and controls. The plates were read at OD₄₁₀ after 1 h incubation with the phosphatase substrate. The experiments were made in duplicate and the error bars indicate range of data.

FIG. 12, panels A-D show in vitro biologic activity of cytokines fused to the antibodies. IL-12 activity (panels A and B) was assessed by determining the [3H]-thymidine incorporation in PHA-activated PBMCs incubated in the presence of varying concentrations of IL-12 fusion proteins. IL-2 activity (panel C) was determined by [3H]-thymidine incorporation in CTLL-2 cells incubated in the presence of varying concentrations of IL-2 fusion proteins. GM-CSF activity (panel D) was determined by MTS/PMS present in the supernatant of FDC-P1 cells incubated in the presence of varying concentrations of GM-CSF fusion proteins. Serial dilutions of anti-HER2/neu IgG3, (IL-12)-IgG3, IgG3-(IL-2), IgG3-(GM-CSF), IgG3-(IL-2) plus (IL-12)-IgG3, (IL-12)-IgG3-(IL-2), IgG3-(GM-CSF) plus (IL-12)-IgG3, and (IL-12)-IgG3-(GM-CSF) were made in quadruplicate and incubated with the different cell lines for 48 h. The error bars indicate the standard deviation of the determinations.

FIGS. 13A and 13B: IgG1 and IgG2a response to ECD^{HER2}. Groups of 8 BALB/c mice were injected s.c. in the right flank with PBS, 8 μg of ECD^{HER2} or with, 8 ∝g of ECD^{HER2} complexed with, either 7 μg of IgG3, or with equivalent molar quantities of IgG3-(IL-2), IgG3-(GM-CSF), (IL-12)-IgG3, (IL-12)-IgG3 plus IgG3-(IL-2), (IL-12)-IgG3 plus IgG3-(GM-CSF), (IL-12)-IgG3-(IL-2), or (IL-12)-IgG3-(GM-CSF) at week 0 and boosted in the same flank at week 5. 2 days before the tumor challenge at week 8, blood samples were collected from each mouse and the serum tested for the presence of anti-(ECD^{HER2}) IgG1 (FIG. 13A) and IgG2a (FIG. 13B) by ELISA. IgG1 titers were determined using sera initially diluted 1:1000 and further diluted 1:3. IgG2a titers were determined using sera initially diluted 1:100 and further diluted 1:2. Values represent the average of duplicate dilutions of serum required to yield an absorbance OD₄₁₀>0.045 at 1 h. The tables at the bottom of FIGS. 13A and 13B include the values of the median for each condition and the significance in the difference between the bi-AbFPs or the mixture of two mono-AbFPs vs. their different controls, with p<0.05 (bold numbers) regarded as significant; ↑ indicates significantly higher titers, and ↓ indicates significantly lower titers.

FIG. 14: Kinetics of tumor growth in vaccinated mice challenged with TUBO. Groups of 8 BALB/c mice were immunized s.c. with either PBS, ECD_{HER2} or ECD_{HER2} combined with IgG3, IgG3-(IL-2), (IL-12)-IgG3, (IL-12)-IgG3 plus IgG3-(IL-2), (IL-12)-IgG3-(IL-2), IgG3-(GMCSF), (IL-12)-IgG3 plus IgG3-(GM-CSF), or (IL-12)-IgG3-(GM-CSF) at week 0 and boosted in the same flank at week 5. Three weeks later the mice were challenged s.c. on the opposite flank with 10⁶ TUBO cells. Tumor size was measured with a caliper every 3 days and tumor volume calculated. The number of tumor-free animals at day 250 is indicated at the bottom right corner of each graph.

FIG. 15: Tumor latency and survival of vaccinated mice challenged with TUBO. The data are from the experiment shown in FIG. 4. Tumor latency is shown in panel A, where ∘ represents animals with visible tumor and ● animals without visible tumors after challenge. Survival is shown in panel B, where ∘ denotes animals that succumbed to tumor challenge and ● animals alive. Mice with tumors=1.5 cm in diameter at the time of inspection were euthanized and considered to have not survived the challenge.

### DETAILED DESCRIPTION

I. Antibody-Immunostimulant Chimeric Moieties as Adjuvants.

A) Monovalent Chimeric Moieties.

The present invention pertains to the use of antibody-immunostimulant chimers (e.g., fusion proteins) that, in certain embodiments, target a soluble (or another state, see, below) form of a disease-related antigen. The antigen, along with antibody-immunostimulant chimeric moiety acting as its adjuvant (e.g., a substance or molecule acting or helping to increase an immune response), elicits an immune response (humoral and/or cellular) within the subject directed against the disease-related antigen (e.g., such as those present on tumor cells, on infectious organisms, etc.). Thus, an “effective adjuvant of a disease related antigen” (e.g., the antibody-immunostimulant chimerica as described herein) is one which produces the desired effect of eliciting an immune response within a subject directed against that antigen and/or against cells, tissues, organisms, etc., bearing that antigen (e.g., as described herein).

In certain embodiments, the chimeric moieties of this invention comprise “monovalent” moieties in which a single antibody is attached to a single species of immunostimulatory molecule (e.g. IL-2, GM-CSF, IL-12, etc.). The antibody, e.g., an intact (full) antibody, an antibody fragment (e.g. Fab, etc.), a single chain antibody, and the like, and be chemically conjugated to the immunostimulatory molecule directly or through a linker. In certain embodiment, the chimeric moiety can be expressed as a fusion protein where the antibody is directly attached to the immunostimulatory molecule or is attached through a peptide linker.

Without being bound by a particular theory, it is believed that the subject's immune response is elicited by the chimeric moiety binding the target antigen (e.g., a disease-related antigen) to form an antibody-antigen immunocomplex (see, e.g., FIG. 2). Of course, such optional mechanism of action should not be construed as limiting. Other possible and/or additional mechanisms of action optionally are used by the methods and compositions of the invention. It is believed that, in certain embodiments, this immunocomplex delivers the disease-related antigen to a dendritic cell (DC) or to another appropriate antigen presenting cell (APC) through the interaction of the antibody-immunostimulant chimeric moiety (e.g. fusion protein) with surface receptors on the DC or APC such as GMCSF, IL-2, IL-12 receptors, etc. (see, e.g., FIG. 2). Depending upon, for example, the specific immunostimulant molecule used in the chimeric moiety (e.g., the specific cytokine, chemokine, etc.) the presentation of the antigen to the DC or APC can in certain embodiments lead to an activation (e.g., a potent activation) of one or both arms of the immune response, i.e., cellular (T_{H}1) and/or humoral (T_{H}2) response. Such activation typically produces a significant immuno-protective activity against the specific disease related antigen (e.g., when the vaccinated subject is challenged with the same, or even, in some embodiments, a closely related disease related antigen).

In yet other embodiments, the immune response produced by the current invention comprises a response that is immuno-protective against the disease and/or pathology characterized by the disease-related antigen (e.g., when the vaccinated subject is re-challenged). In yet other embodiments, the invention produces a detectable change in immune status (e.g., a change in cellular and/or humoral immune levels or response against the disease related antigen). Also, in certain embodiments, the chimeric moieties of the invention target a dead or dying cell (such as an apoptotic cell) or cell fragment (e.g., where the cell/cell fragment arises from a disease related state, such as a cancer cell, etc. and comprises one or more disease-related antigen(s)). Thus, in certain embodiments, the chimeric moieties of this invention binding the cell/cell fragment can make it easier for the cell/cell fragment to be phagocitized by other cells (e.g., APC) and can improve trafficking and presentation in APCs.

B) Bivalent Chimeric Moieties.

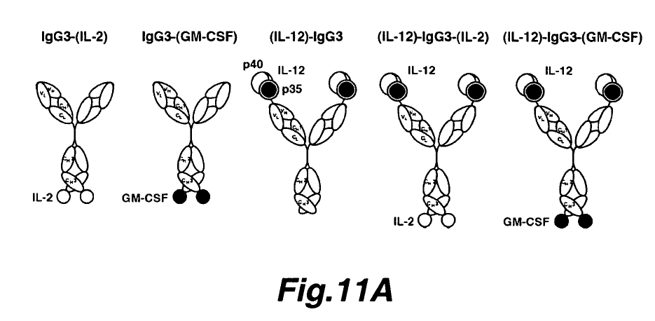

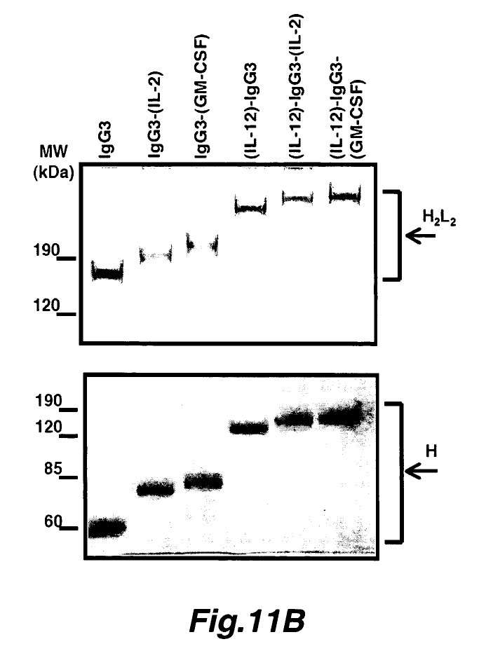

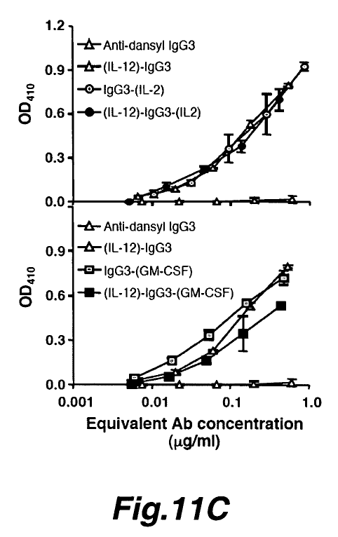

In certain embodiments, the chimeric moieties of this invention comprise “bivalent” moieties in which a single antibody is attached to two different species of immunostimulatory molecules (e.g. IL-2, GM-CSF, IL-12, etc.). Thus for example, we have constructed bi-functional antibody-cytokine chimeras (e.g., fusion proteins) specific for the extracellular domain of the human tumor associated antigen HER2/neu (ECDHER2). These antibody fusion proteins are composed of human IgG3 [containing the variable region of trastuzumab (Herceptin, Genentech, San Francisco, Calif.)], genetically fused to the immunostimulatory cytokines interleukin-2 (IL-2) and interleukin-12 (IL-12) or granulocyte-macrophage colony stimulator factor (GM-CSF) and interleukin-12 (IL-12) (see, e.g., FIG. 11A) in which they are compared with the antibody fusion proteins fused to only one cytokine (mono-functional antibody-cytokine fusion proteins).

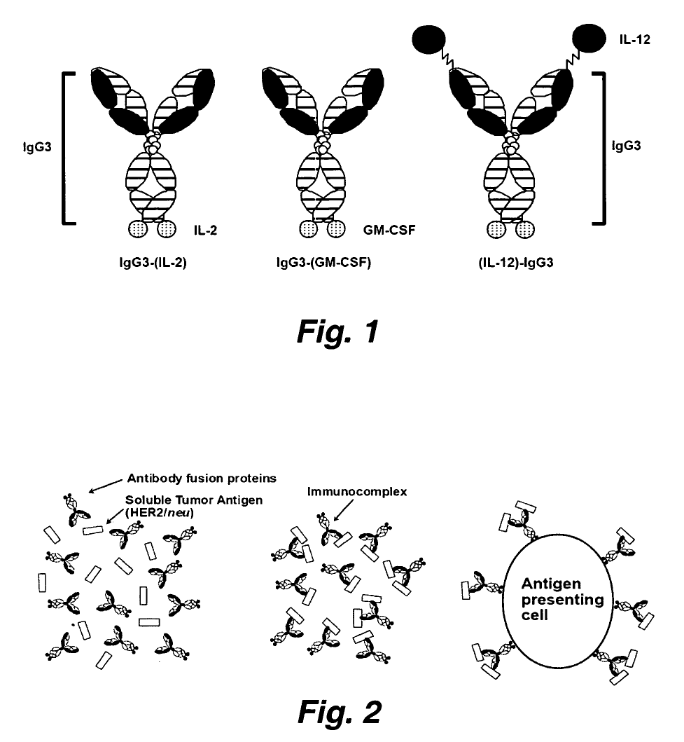

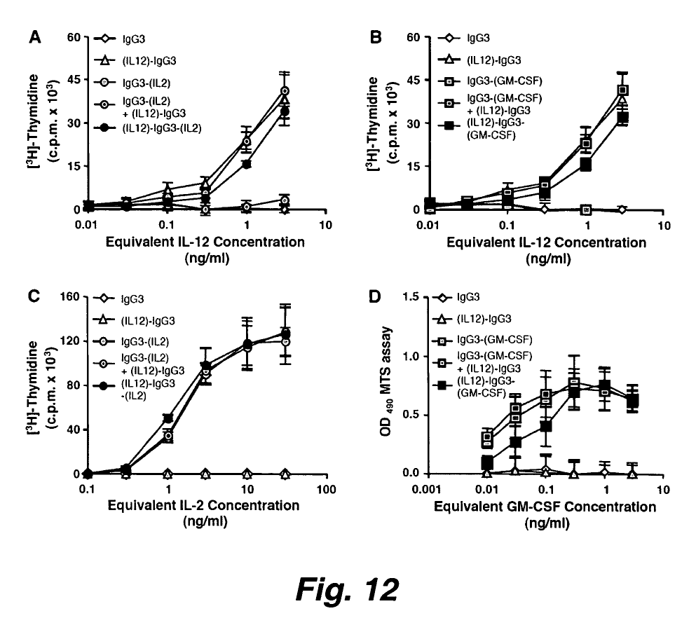

These novel fusion proteins [anti-HER2/neu (IL-12)-IgG3-(IL-2) and anti-HER2/neu (IL-12)-IgG3-(GM-CSF)], expressed in murine myeloma cells, had the expected molecular weight and were properly assembled and secreted (FIG. 1B Helguera et al., Vaccine, in press). They bind antigen and (FIG. 1C Helguera et al., Vaccine, in press) carry out cytokine activities (see, e.g., FIG. 12).

The bivalent chimeric moieties of this invention represent both a novel technology and a product with a novel application. The chimeric moieties (e.g., anti-HER2/neu (IL-12)-IgG3-(IL-2), anti-HER2/neu (IL-12)-IgG3-(GM-CSF), etc.) can readily be used as as potent adjuvants (i.e. immunoenhancers) to direct an immune response to a target antigen and/or to cells, tissues, pathogens, etc. bearing such an antigen (e.g. to a soluble form of HER2/neu in the case of certain cancers).

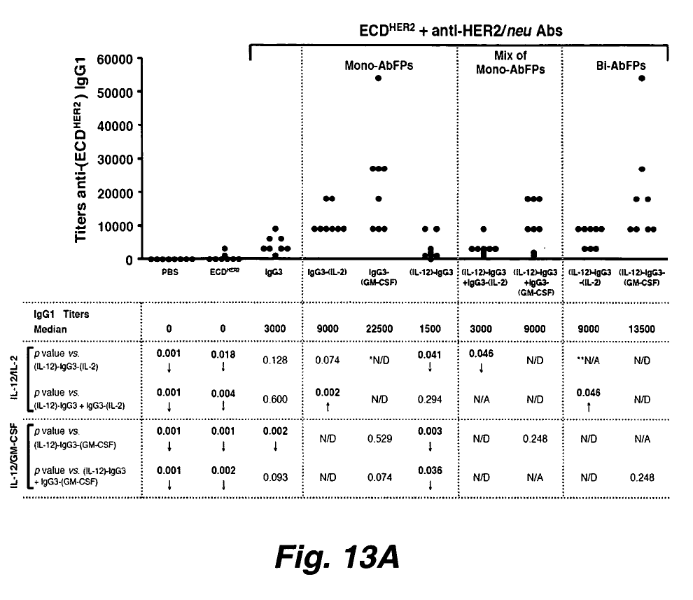

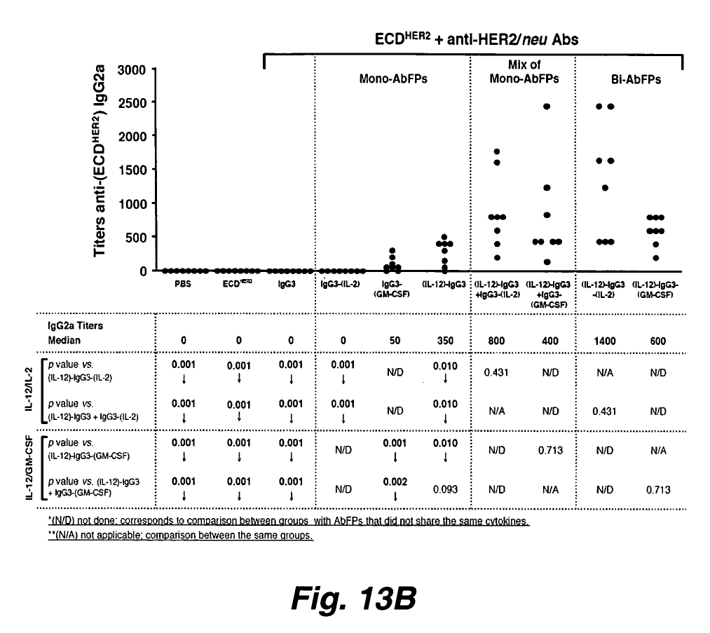

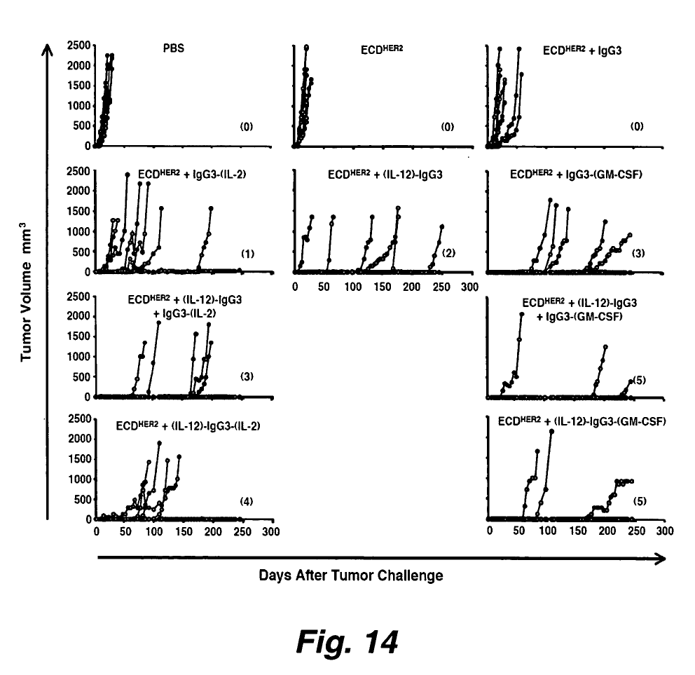

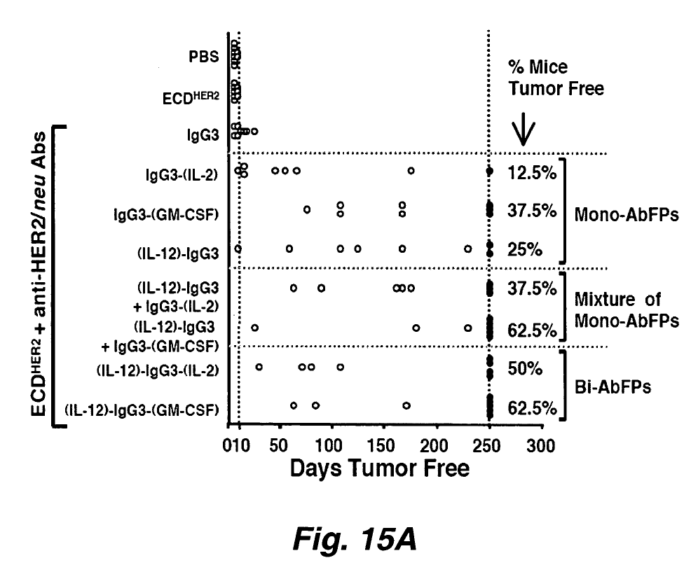

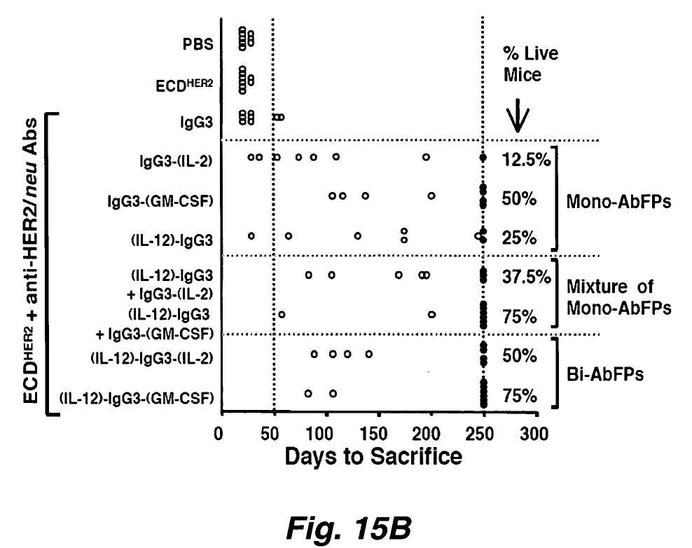

As shown, e.g. in Example 3, the combination of single chain IL-12 and IL-2 or GM-CSF in a single antibody such as IgG3 did not result in loss of activity of either component. Moreover, mixing the bi-functional anti-HER2/neu antibody-cytokine fusion protein with the extracellular domain of HER2/neu (ECDHER2) was enough to elicit a potent cellular and humoral immune response (see, e.g., FIG. 13) that would result in a stronger antitumor activity compared to the use of mono-functional antibody-cytokine fusion protein alone (see, e.g., FIGS. 14 and 15 and Table 4 in Example 3).

In the present approach, tumor targeting by antibodies (or antibody fusion protein) is not a requirement to trigger an antitumor activity. The data provided herein indicate that both humoral and cell-mediated responses contribute to the enhanced antitumor activity. The data also indicate that the bi-functional anti-HER2/neu antibody-cytokine fusion proteins can be effective prophylactic and therapeutic agents against HER2/neu expressing tumors in patients.

In the embodiments illustrated in Example 3, murine GM-CSF and murine IL-12 were used because human GM-CSF and human IL-12 are not active in mice. Using murine GM-CSF and IL-12 in the chimeric moieties allowed testing of the invention in murine models. In chimeric constructs for use in humans, the human form of the immune enhancer (e.g. IL-12, GM-CSF, etc.) would typically be used. Similarly, while the illustrated chimeric moieties used human IgG3, any isotype can be used. Moreover, the chimeric moieties can utilize other kinds of antibodies including, but not limited to single chain antibodies (e.g. scFv, scFab′, etc.), antibody fragments, and the like.

The bi-functional antibody-cytokine fusion proteins would serve as novel molecules and strategy (i.e. as an adjuvant of soluble antigens such as the tumor associated antigen ECDHER2) in prophylactic or therapeutic vaccinations against targets expressing the antigen such as HER2/neu expressing tumors.

It is demonstrated herein that immunization of mice with (ECDHER2) plus bi-functional antibody-cytokine fusion proteins results in a potent activation of both arms of the immune response: cellular (Th1) and humoral (Th2) and that this activation is associated with a significant antitumor activity when immunocompetent mice were challenged with HER2/neu expressing tumors. Importantly, this antitumor activity is superior compared to the isolated use of the parental mono-functional fusion proteins.

Without being bound by a particular theory, it is believed that the chimeric moieties described herein target and deliver ECDHER2 into dendritic cells (DCs), or into other antigen presenting cells (APCs), through the interaction of the antibody-cytokine chimeric moiety with DCs surface receptors such as GM-CSF, IL-2, IL-12 receptors.

Using in vitro culture of DCs we found that anti-HER2/neu IgG3-(IL-2) and anti-HER2/neu IgG3-(GM-CSF) mono-functional fusion proteins promote HER2/neu processing and presentation, under conditions in which the anti-HER2/neu IgG3 (antibody without the fused cytokine) or a non-HER2/neu specific IgG3-(IL-2) or IgG3-(GM-CSF) failed to promote antigen processing and presentation. Therefore, a physical non-covalent association between the antibody-cytokine fusion protein and the antigen appeared to be important to achieve proper antigen presentation in our in vitro studies. In addition, using a mouse model we clearly demonstrated that a physical association between ECDHER2 and anti-HER2/neu IgG3-(IL-2) or anti-HER2/neu IgG3-(GM-CSF) fusion proteins elicited the most effective anti-tumor immunity). These results indicated that immunocomplexes between the soluble antigen and the antibody fusion proteins appear to elicit optimal immunoactivation and immunoprotection. This mechanism is illustrated in FIG. 2.

Since the bi-functional chimeric moieties (e.g. antibody fusion proteins) have two different cytokines instead of one, it is believed that they are much more effective in targeting and internalizing the bound antigen in to APCs through their respective cytokine receptors resulting a much stronger immune response compared to the use of mono-functional fusion proteins. It is also possible that the interaction of the Fc fragment of the antibody with the Fc gamma receptor of DCs (or other APCs) may be important to elicit optimal antigen presentation empowering the effect of the interactions through the two cytokine receptors. In addition, in certain embodiments, the bi-functional chimeric moieties contain combinations of immunostimulatory cytokines with complementary activity (GM-CSF-APC activation) and (IL-12-deviation to TH1), or IL-2—(cell proliferative signal) and (IL-12-deviation to TH1) which results in the orchestration of a combined (cellular and humoral) and potent immune response typically not achieved with the single use of mono-functional fusion proteins.

In certain embodiments, where the chimeric moiety is directed against HER2/neu, the elicited immune response is expected to be against the HER2/neu expressed on the surface of cancer cells (humoral immune response) as well as against HER2/neu peptides associated with MHC class I on the surface of cancer cells (cellular immune response). In addition, as HER2/neu has high homology with other growth factor receptors such as epidermal growth factor receptors 1, 2, and 3 (EGF1, EGF2, EGF3), we believe the elicited immune response (humoral and/or cellular) is directed not only against the targeted antigen (HER2/neu), but also against other homologous receptors that are expressed on the cancer cell.

C) Uses of Antibody-Imustimulatory Chimeric Moieties.

The chimeric moieties of this invention can be used to enhance an immune response against an antigen, and/or a cell or tissue displaying, expressing, or shedding that antigen, and/or a pathogen expressing, displaying, shedding, or inducing production of that antigen, and the like. The chimeric moieties can be used in a therapeutic treatment and/or as a prophylactic. Thus, in certain embodiments, this invention contemplates, for example, prophylactic vaccination of subjects at high risk to develop tumors (e.g. subjects expressing high levels of HER2/neu, BRAC2, and the like). In various embodiments the subjects would be vaccinated with the chimeric moiety and/or with the chimeric moiety in conjunction with the antigen.

In various therapeutic embodiments patients undergoing a particular pathology, e.g., cancer, bacterial infection, viral infection, fungal infection, parasite infection, and the like, will be treated with the chimeric moiety comprising an antibody that binds a characteristic antigen involved in the pathology and/or with a combination of the chimeric moiety and the antigen. Thus, for example, patients bearing tumors expressing HER2/neu can be vaccinated with a mixture of bi-functional antibody-cytokine fusion protein/s and the soluble antigen (HER2/neu).

In the case of patients bearing tumors expressing HER2/neu and showing circulating soluble HER2/neu antigen (due to tumor shedding of ECDHER2) it is expected that treatment with bi-functional antibody-cytokine fchimera alone can be enough to target the patient's soluble form of HER2/neu (ECDHER2) and in so doing trigger the desired immune response. However, if convenient those patients can also be treated with a mixture of antibody-cytokine fusion protein/s and the soluble antigen (HER2/neu).

It is also possible to use two or more different bi-functional antibody-cytokine fusion proteins in combination seeking an additive or a synergistic effect.

The chimeric moieties of this invention need not necessarily be a replacement of available therapeutic (e.g., anti-HER2/neu) technologies such as the recombinant antibody trastuzumab (Herceptin, Genentech, San Francisco, Calif.), but instead provide an alternative therapy to be used in combination with that antibody or other anti-cancer approaches such as chemotherapy and/or radiotherapy. Importantly, patients with high levels of circulating shed ECDHER2 or with mutated form of HER2/neu, who do not respond to the treatment with trastuzumab may receive a lot of benefit. In addition, our approach may also be effective for ex vivo generation of mature DCs. In this case DCs obtained from patients will be treated (in vitro) with a mixture of antibody-cytokine fusion protein/s and the soluble antigen. Then, the mature and programmed DCs will be re-implanted into the patient.

The antigen (e.g. disease-related antigen) for which the antibody immunostimulant chimeric moiety acts as an adjuvant need not be a soluble antigen, though that is often the case in many embodiments. Other embodiments comprise chimeric moieties (e.g., antibody-immunostimulat fusion proteins) where the antigen for which the antibody-immunostimulant fusion protein acts as an adjuvant includes, but is not limited to antigen(s) (soluble or insoluble) bound to a matrix such as a bead, etc., an insoluble aggregate of antigens or aggregate of soluble antigens (both of which could also comprise other materials, e.g., to help in aggregation, etc., non-viable cell associated antigens (e.g., also including non-viable organismal associated antigens such as form bacteria, viruses, etc., antigens conjugated with liposomes, etc. Additionally, in yet other embodiments, the antibody-immunostimulant fusion protein which acts as the adjuvant to the antigen may itself be conjugated with, e.g., a liposome, etc. while the antigen is, or is not, so conjugated to a liposome.

In various embodiments the immune response elicited by the methods and compositions of the invention are specific against the disease related antigen present within (or, if used in prophylactic treatment, expected or possibly expected within) the subject or antigenically closely related molecules. Thus, for example, one embodiment of the invention optionally comprises an anti-tumor associated (TAA) antigen antibody-immunostimulant and a soluble disease related antigen (e.g., here the TAA) used as a therapeutic treatment. The immune response elicited by such treatment can be directed against such antigen (or a closely related antigen) present on, e.g., the cell surface of tumors present within the subject.

The chimeric moieties of this invention act as adjuvants to disease related antigens (e.g., tumor antigens presented by or on tumor cells or shed from tumor cells such as HER2/neu, or antigens presented by or on an infectious organism such as a virus, a bacteria (e.g., a protein A antigen from Staphylococcus aureus), a fungus, a prion, a parasite, an autoimmune disorder, etc.). The current invention utilizes the humoral and/or cellular immune response generated by the disease related antigen (and its antibody-immunostimulant adjuvant) as a means of therapeutic and/or prophylactic treatment of the subject against the organism or disease which generated or caused the disease related antigen's presence in the subject.

Again, it should be noted that the current invention encompasses a myriad of chimeric moieties and their uses against a myriad of diseases/conditions. In many examples herein, the anti-HER2/neu antibody fusion, etc. is used as one example, but such should not be construed as limiting. Discussion of HER2/neu protection, etc. is to illustrate the general concepts of the methods and compositions of the invention, namely that use of an antibody-immunostimulant fusion protein as an adjuvant of an antigen vaccination leads to humoral and/or cellular immune response in a subject and thus can be used as a therapeutic and/or prophylactic treatment of the subject for the disease or infection which presents such antigen.

The interaction of the antibody-immunostimulant fusions and the disease-related antigen with the APC or DC, as illustrated in FIG. 2 could change the quantity and/or quality of antigen presentation (e.g., from that which would occur with solely the disease related antigen used in treatment), which could result in (depending again upon, e.g., the specific immunostimulant fused with the antibody) a strong T and/or B cell immune response against the disease related antigen. Additionally, the general immunostimulatory activity of many immunostimulants (e.g., of cytokines) which are fused to the antibody fusion proteins of the invention may also contribute to the enhancement of the immune response against the targeted antigen (e.g., IL-2-cell proliferative signal, GMCSF-APC activation and IL-12-deviation to T_{H}1, etc.). Again, such mechanisms of action should not be construed as limiting. The efficaciousness of the methods and compositions of the invention are not limited to only these mechanisms of action.

The elicited immune response (i.e., produced through use of the methods, etc. of the present invention) is, in certain embodiments, directed against the disease related antigens expressed on the surface of, or shed by, e.g., cancer cells or infectious agents (humoral immune response) as well as against disease related antigen peptides associated with MHC class I on the surface of tumor cells or infectious agent cells, etc. (cellular immune response). In some embodiments, the current invention additionally elicits humoral and/or cellular immune responses against other closely related antigens (e.g., antigens closely related either structurally or conformationally to the antigen used as the protein vaccination). For example, since HER2/neu has high homology with other growth factor receptors such as epidermal growth factor receptors 1, 2, and 3 (EGF1, EGF2, EGF3), the elicited immune response (humoral and/or cellular) from the invention against HER2/neu is optionally directed not only against the targeted disease related antigen (HER2/neu), but also against other homologous receptors that are expressed on a cancer cell.

In some embodiments, the methods, etc. of the current invention (as well as the toxicological studies, use studies, etc. of the current invention) are carried out in animal models (see, e.g., Examples I and II below), however, the current invention also encompasses embodiments where human subjects are utilized (including clinical trials, etc.). In humans, as in other animal subjects, the antibody-immunostimulant fusion proteins serve as an adjuvant of, e.g., a soluble antigen in both, prophylactic or therapeutic vaccinations. Thus, the invention can target patients with specific antigen expressing tumors, e.g., HER2/neu breast cancers, etc. as well as disease-related antigens presented by infectious organisms (viruses, bacteria, etc.) both where the tumor/infectious organism, etc. is within a subject (therapeutic) or before such disease/infection arises in a subject (prophylactic). Thus, the applications allowed by the methods and compositions of the invention comprise a broad range of treatments for both human and other animals in protection against numerous disease states, including cancers and infection by microorganisms.

For example, in prophylactic vaccination, patients at high risk to develop tumors (e.g., those tumors that express HER2/neu) are optionally vaccinated with a mixture of antibody-immunostimulant fusion protein and an appropriate tumor antigen (e.g., HER2/neu, etc.). For example, women whose family history indicates a high probability of developing breast cancer are optionally prophylactically treated with an embodiment of the current invention. For example, antibody-immunostimulant fusion proteins comprising an antibody specific for HER2/neu fused with, e.g., IL-2, IL-12, and GMCSF (i.e., in different antibody constructs) are optionally administered to the woman along with an appropriate amount of HER2/neu antigen (see, below). Typically such fusion proteins and antigens are incubated together in order to form the appropriate immuno-complexes before administration to the subject. The use of the invention would thus cause the woman's immune system to develop an immune response against the HER2/neu protein and thus the woman would be better able to more effectively combat any HER2/neu expressing cancers that arose, and would optionally increase her chances of long-term survival.

Again, it should be noted that in other embodiments of the invention, different antibody/immunostimulant combinations are used against different diseases/conditions and thus against different antigens, etc. Thus the current invention is also optionally used to prophylactically treat subjects for exposure to particular viruses, bacteria, etc. For example, the current invention is optionally used to prophylactically treat persons such as health care workers who might be in environments where risk of exposure to particular viruses/bacteria is high. For example, health care workers likely to be exposed to, e.g., S. aureus contamination are optionally prophylactically treated with an anti-protein A antibody-immunostimulant fusion protein and the protein A antigen (see, e.g., Example II below for a similar example with mice). Alternatively, persons likely to encounter, e.g., certain viruses (e.g., such as HIV for sex workers, etc.) are optionally prophylactically treated with an appropriate antibody-immunostimulant fusion specific for an appropriate HIV antigen along with that particular antigen.

In therapeutic treatment vaccinations, patients, e.g., those bearing tumors expressing a particular antigen are vaccinated with a mixture of antigen-specific antibody-immunostimulant fusion protein(s) and the antigen(s) (optionally, the soluble antigen, see above). Again, therapeutic vaccinations are applicable to, e.g., myriad tumor types (and to different antigens presented on the same tumors) and to therapeutic treatment of various infections such as viral, bacterial, etc. So, similarly to a prophylactic treatment (see, above) the antigen targeted can be tumor associated, virus associated, bacterial associated, etc. Therapeutic treatment using the methods and compositions of the invention are especially useful in situations where the subject is having difficulty mounting an effective immune response against the disease state. For example, when disease related antigens are not being appropriately interacted with APCs, etc. or when the disease related antigens are recognized as “self” by the immune system, etc.

In some situations, it should be noted, patients will present disease profiles where high levels of the specific targeted antigen are present within the patient. For example, some tumors express high circulating levels of soluble antigen (due to, e.g., tumor shedding of the antigen). Such is the case with some HER2/neu expressing tumors; the tumors shed high levels of the antigen. Additionally, in some infections, high levels of a targeted antigen can be present in the patient. Some, e.g., bacterial infections can result in high levels of innately present antigen which is thus able to be targeted. For example, various septicemias can optionally present high levels of soluble antigen in a subject's blood stream. Therefore, in some cases the injection of antibody-immunostimulant fusion protein(s) alone is enough to target the desired antigen. In other words the patient's innate levels of antigen, e.g., soluble HER2/neu, bacterial antigen, etc. are high enough to be targeted by the antibody-immunostimulant fusion proteins and thus trigger the desired immune response. However, even if high levels of innate antigen exist, such patients can also optionally still be injected with a mixture of the antibody-immunostimulant fusion protein(s) and the targeted antigen.

The different antibody-immunostimulant fusion proteins and antigens herein can be used separately or in combination, thus creating an additive or a synergistic effect. In various embodiments of the invention, different immunostimulant domains are optionally used with the same antibody framework (i.e., the same antibody against the same antigen—see, as with the different fusions in Example I, below). Alternatively, and/or additionally, multiple antigens (e.g., two different surface antigens on a bacterial cell, mycoplasm, etc. or two different tumor associated antigens) are optionally used (i.e., the different antigens each have one or more antibody-immunostimulant fusion protein made to target them). Thus, various layers of fine-tuning and specificity are built into the current invention, which thus allow more precise control and targeting of disease treatment in subjects.

Additionally, the methods of the current invention (e.g., as illustrated by treatment with anti-HER2/neu antibody fusion proteins, etc.) are not necessarily a replacement of available therapeutic technologies such as the recombinant antibody Trastuzumab (Herceptin, Genentech, San Francisco, Calif.) treatment. Instead, the current invention is optionally used as an alternative therapy in combination with other treatments (e.g., anti-cancer approaches such as chemotherapy and/or radiotherapy, antibiotics, etc.). For example, in some situations patients with high levels of circulating antigen (e.g., as is seen with tumors that shed ECD^{HER2}) or with mutated forms of an antigen (e.g., a mutated form of HER2/neu) who do not respond to treatment with Trastuzumab (see, e.g., Baselga et al., 1996 “Phase II study of weekly intravenous recombinant humanized anti-p185HER2 monoclonal antibody in parities with HER2/neu-overexpressing metastatic breast cancer” J Clin Oncol 14:737-44), optionally can benefit from the methods, etc. of the current invention.

In addition, some embodiments of the current invention are also effective for ex vivo generation of mature dendritic cells. In such case, dendritic cells obtained from subjects are treated (in vitro) with a mixture of antibody-immunostimulant fusion protein(s) and the appropriate soluble (or other format, such as antigen on latex beads, etc.) antigen. Then, the mature and programmed dendritic cells are re-implanted into the patient. This is similar in some aspects to some optional embodiments above, i.e., the antibody-immunostimulant complexes form and interact with an APC, etc., but here, such interaction occurs ex vivo.

II. Components and Construction of Antibody-Immunostimulant Fusion Proteins and Antigen Vaccines

The chimeric moieties of this invention typically comprise an antibody or antibody domain attached to one or more immunostimulatory molecule(s)/component(s). In various embodiments the chimeric moiety functions as an effective adjuvant of a disease related antigen.

While certain embodiments illustrated herein describe antibodies directed to the HER2/neu tumor antigen (anti-HER2/neu antibodies) attached to IL-2 and/or IL-12 and/or GMCSF, the present invention encompasses other combinations of immunostimulant molecules and antibodies. Depending upon the specific condition/disease being considered or treated, various combinations of immunostimulant molecules (e.g., cytokines, chemokines, adjuvants, superadjuvants, etc.) and antibodies (e.g., different antibody fragments, antibodies of different isotype, and different antibodies with specificity against different antigens) are encompassed within the current invention. Various mono-valent and bi-valent (mono-functional and bi-functinoal) chimeric moieties are schematically illustrated in FIGS. 1A and 11A.

The chimeric moieties can comprise antibodies chemically conjugated to one or more immunostimulant molecules (e.g. directly or via one or more linkers) or, in certain embodiments, the chimeric moiety is a fusion protein (e.g., a recombinantly expressed fusion protein). In certain chemically conjugated constructs, a single linker can be used to join an antibody to one or, optionally, to immunostimulant molecules, or separate linkers can be used to join an antibody to each of two different immunostimulat molecules or the two immunostimulant molecules can be joined to each other and to the antibody. In either chemically conjugated moieties or fusion proteins, the order of immunostimulant and antibody is not critical. Thus, for example, several variations are illustrated below:

1) immunostimulant 1—antibody—immunostimulant 2

2) immunostimulant 1—immunostimulant 2—antibody

3) antibody—immunostimulant 1—immunostimulant 2

This list is intended to be illustrative and not limiting.

In certain embodiments the chimeric construct can be complexed with the antigen that is typically bound by the antibody to form an antigen/chimeric moiety complex.

In addition to the variability of the immunostimulant domain(s) of the chimeric moieties described herein, the specific antibody domain used can also vary. The antibody domains utilized in the examples herein are not to be construed as limiting. For example, different antibodies (e.g., against bacterial antigens, against viral antigens, against different tumor associated antigens, against mycoplasm antigens, against antigens of parasites, prions, autoimmune disorders, etc.) are all various embodiments of the current invention.

This the ability to alter the antigen specificity allows the methods and compositions of the present invention to be used to treat and/or prevent myriad specific conditions, disease states, etc. Not only is the antigen specificity of the antibody domain variable, but the type of antibody framework which comprises the protein fusion can vary as well. For example the antibody domain of the antibody fusion proteins herein can optionally comprise Fab, Fab′, F(ab)₂, F(ab′)₂, Fv, scFv, an antibody fragment, and various combinations thereof, etc.

A) Antibodies

The present invention utilizes chimeric moieties comprising an antibody attached to one or two (or, in certain embodiments three or more) immunostimulant molecules as adjuvants to direct an immune response against a particular antigen, and/or cells and/or tissues and/or pathogens (e.g. bacteria, virus, mold, fungus) participating the the production of, and/or bearing, and/or shedding the antigen. In typical embodiments, the antibody is specific for a disease related antigen.

The antibody domain of the fusion protein optionally comprises all or part of an immunoglobin molecule and in certain embodiments, contains all or part of an immunoglobin variable region (i.e., the area of specificity for the disease related antigen) and optionally comprises region(s) encoded by a V gene, and/or a D gene and/or a J gene.

In various embodiments the antibodies used in the chimeric moieties of this invention can include, but are not limited to F(ab)₂, F(ab′)₂, Fab, Fab′, single chain (e.g. scFV, scFab′, etc.) and the like. Some embodiments utilize antibodies comprising IgG domains. Other embodiments utilize antibodies comprising alternate immunoglobins of immunoglobin domains including, but not limted to IgM, IgA, IgD, IgE, etc. Furthermore, all possible isotypes of the various immunoglobins are also encompassed within the current embodiments. Thus, IgG1, IgG2, IgG3, etc. are all possible molecules in the antibody domains of the antibody-immunostimulant chimeric moieties used in the invention. In addition to choice in selection of the type of immunoglobin and isotype, different embodiments of the invention can comprise various hinge regions (or functional equivalents thereof). Such hinge regions provide flexibility between the different domains of the antibody-immunostimulant moieties.

Typically antibodies for use in the chimeric moieties of this invention are selected for their ability to bind, and preferably to specifically bind a particular target antigen, often a disease-related antigen. In various embodiments the antibody is selected to bind target antigen with an affinity or avidity (K_{D} of at least 10⁻⁶ M, preferably at least 10⁻⁷ M, more preferably at least 10⁻⁸ M or 10⁻⁹ M, and most preferably at least 10⁻¹⁰, at least 10⁻¹¹, or at least 10⁻¹², 10⁻¹³, or 10⁻¹⁴ M.

In certain embodiments the antibody is slected to specifically bind a disease-related antigen. The antigen can be an antigen characteristic of a partuclar cancer, and/or of a partuclar cell type (e.g. a hyperproliferative cell), and/or of a particular pathogen (e.g. a bacterial cell (e.g. tuberculosis, smallpox, anthrax), a virus (e.g., HIV), a parasite (e.g. malaria, leichmaniasis, etc.), a fungal infection, a mold, a mycoplasm, a prion antigen, an autoimmune disorder antigen, and the like).

With respect to developing an enhanced immune response against cancers (e.g., solid tumors, cancer cells, etc.), it is noted that a number of cancer-specific markers (antigens) are known to those of skill in the art. Such markers include, but are not limited to C-myc, p53, Ki67, erbB-2, Her2, Her4, BRCA1, BRCA2, Lewis Y, CA 15-3, G250, HLA-DR cell surface antigen, CEA, CD2, CD3, CD7, CD19, CD20, CD22, integrin, EGFr, AR, PSA, carcinoembryonic antigen (CEA), the L6 cell surface antigen (see, e.g., Tuscano et al. (2003) Neoplasia, 3641-3647; Howell et al. (1995) Int J Biol Markers 10: 126-135; Marken et al. (1992) Proc. Natl. Acad. Sci. U.S.A. 89: 3503-3507, 1992), certain growth factor receptors, and the like.

In certain embodiments, the antibodies can be selected that bind antigens associated with breast cancer, such as epidermal growth factor receptor (EGFR), fibroblast growth factor receptor, erbB2/HER-2 and tumor associated carbohydrate antigens (Siegall (1994) Cancer, 74(3): 1006-1012). CTA 16.88, homologous to cytokeratins 8, 18 and 19, is expressed by most epithelial-derived tumors, including carcinomas of the colon, pancreas, breast, ovary and lung. Thus, antibodies directed to these cytokeratins, such as 16.88 (IgM) and 88BV59 (IgG3k), which recognize different epitopes on CTA 16.88 (Jager et al. (1993) Semin. Nucl. Med., 23(2): 165-79), can be utilized in the chimeric moieties of this invention. For enhancing an immune response against colon cancer, anti-CEA antibodies, can be employed. The MG series of monoclonal antibodies can be used for enhancing an immune response against, for example, gastric cancer.

There are a variety of cell surface epitopes on epithelial cells for which the antibodies can be selected to develop an enhanced immune response. For example, the protein human papilloma virus (HPV) has been associated with benign and malignant epithelial proliferations in skin and mucosa. Two HPV oncogenc proteins, E6 and E7, may be targeted as these may be expressed in certain epithelial derived cancers, such as cervical carcinoma (see, e.g., (1994) Curr. Opin. Immunol. 6(5: 746-754). Membrane receptors for peptide growth factors (PGF-R), which are involved in cancer cell proliferation, can also be selected as tumor antigens (see, e.g, (1994) Anticancer Drugs, 5(4): 379-393). In certain embodiments, antibodies directed against certain melanoma associated antigens (MAA) such as adhesion molecules ((1994) Tumor Biol., 15(4): 188-202), which are expressed by malignant melanoma cells, can used be used in the chimeric moieties of this invention. The tumor associated antigen FAB-72 on the surface of carcinoma cells can also be selected. These antigens are intended to be illustrative and not limiting.

As indicated herein, the chimeric moieties of this invention are not typically used to target (e.g. bind to) a particular target cell, but rather are used as “adjuvants” to enhance an immune response directed to a particuarl disease-related antigen and the cells and/or tissues and/or pathogens displaying or shedding such antigen. Thus, in certain embodiments the antibody is selected to specifically bind a soluble antigen and/or a shed antigen, e.g. an antigen that can be found in circulation. Such a soluble and/or shed antigen can be “free” in solution or suspension and/or associated with various blood components and/or associated with various cellular components/debris. Thus, in certain instances, the shed antigen may remain associated with some cell membrane and/or membrane components.

In some embodiments of the compositions of the invention, the antigen comprises, e.g., a soluble antigen, a soluble antigen bound to a matrix, an insoluble antigen bound to a matrix, an insoluble aggregate of antigens, a nonviable cell-associated antigen, or a nonviable organism-associated antigen, or an antigen conjugated with a liposome.

In various embodiments such antigen(s) can comprise, e.g., HER2/neu (or HER2/neu shed from a tumor cell) or fragments thereof. Additionally, the antigen in such compositions optionally comprises: an antigen other than a tumor antigen, an antigen arising from a subject, an antigen arising from a disease state within the subject, an antigen arising from a disease related organism within a subject (e.g., a disease state caused by one or more of a tumor, a bacteria, a virus, a mycoplasm, a fungus, a prion, an autoimmune disorder, or an infectious parasite such as an infectious parasite of a mammal, etc.). The antigen can also comprise a tumor antigen, a bacterial antigen, a viral antigen, a mycoplasm antigen, a prion antigen, an autoimmune disorder related antigen, or an infectious parasite antigen. In some embodiments herein, the antigen is an exogenous antigen (which is optionally substantially identical to an antigen arising from a subject, or from a disease state within a subject or from a disease related organism within the subject).

While the chimeric moieties are described comprising a single antibody, in certain embodiments, two or more antibodies/antibody domains are provided to direct/enhance an immune response against two or more antigens, thereby, for example, providing greater specificity where particular pathogens and/or pathological cells are characterized by the co-expression and/or co-display of two or more antigens.

In addition, while the chimeric moieties comprising a single antibody joined to one or two immunoistimulatory molecules are illustrated herein, it will be recognized that chimeric moieties comprising two or more copies of the antibody and/or two or more copies of one or both immunostimulatory agents are also contemplated within the scope of this invention.

B) Immunostimulants

In addition to the antibody, the chimeric moieties of theh present invention also include one or two immunostimulatory molecules/domains. As described above, an immunostimulant molecule (or domain) acts to stimulate or elicit an immune response or an action of the immune system of a subject. Immunostimulant domains that are part of the antibody-immunostimulant fusion protein are typically (but not only) of several broad types. Typically, embodiments include, but are not limited to, cytokines and chemokines. In general, cytokines act to, e.g., stimulate humoral and/or cellular immune responses, while chemokines in general induce immune cell migration and activation. The choice of which immunostimulant to include in a particular embodiment depends upon, e.g., which particular immune response effects are desired, e.g., a humoral response, a cellular immune response, or both. In typical embodiments both cellular and humoral immune responses against a disease related antigen are desired. Thus, as illustrated in the Examples herein, multiple chimeric moieties with varying immunostimulant domains can be used in the methods and compositions of the invention.

It will be appreciated that the discussion herein of immunostimulants comprising the listed molecules (e.g., IL-2, etc.) is not to be taken as limiting. In other words, it is to be understood that various embodiments of the invention comprise different combinations of immunostimulant molecules (e.g., other cytokines, chemokines, etc. besides, or in addition to, those listed herein). Thus, specific cytokines/chemokines, etc. (e.g., various interleukin molecules, interferons, IL-2, IL-10, IL-12, IL-17, IL-18, RANTES, mip1α, mip1B, GMCSF, GCSF, gamma interferon, alpha interferon, etc.) in the chimeric moieties described herein are not limiting and different specific cytokines, chemokines, immunostimulants, etc. can be utilized for different applications, all of which are part of the present invention.

For example, one common immunostimulant domain capable of use in the present invention comprises cytokines. Cytokines comprise a large family of growth factors that are primarily secreted from leukocytes and include, but are not limited to, e.g., IL-1, IL-2, IL-4, IL-6, IL-7, IL-10, IL-13, interferons, interleukins, IFNs (interferons), TNF (tumor necrosis factor) and CSFs (colony stimulating factors). Various cytokines can stimulate humoral and/or cellular immune responses in subjects and can activate phagocytic cells. Interleukins are one species of cytokine that are secreted by leukocytes and that also affect the various cellular responses/actions of leukocytes (e.g., IL-2, IL-12, etc.). In various embodiments, interleukins are used as an immunostimulant domain in the methods/compositions of the invention. Additionally, in other embodiments of the invention, non-interleukin cytokines comprise an immunostimulant domain of the chimeric moiety (see, e.g.,., Mire-Sluis (1993) TIBTECH 11:74-77; Colombo et al. (1992) Cancer Res 52:4853-4857; Arai et al, (1990) Annu Rev Biochem 59:783, etc. for discussion of various chemokines and cytokines).

Examples of possible cytokines used in particular embodiments of the present invention include, but are not limited to, IL-2, IL-12, GM-CSF, and the like.

Interleukin 2 (IL-2) stimulates T cells to proliferate and to become cytotoxic. Additionally, IL-2 induces NK cells to respond with increased cytotoxicity toward cells (e.g., tumor cells). Additionally, IL-2 increases vascular permeability leading to the efflux of intravascular fluids into extravascular areas.

Interleukin 12 (IL-12) is normally released by professional antigen presenting cells and promotes cell-mediated immunity. It does so by inducing naïve CD4+ cells to differentiate into T_{H}1 cells. IL-12 also can enhance the cytotoxicity of NK and CD8+ T cells. The IFN-γ produced by T and NK cells that are stimulated by IL-12 can lead to other immune actions as well. IL-12 can exist as single chain or double chain (heterodimers) variants. Either permutation of IL-12 is optionally used herein as an immunostimulant domain in the antibody-immunostimulant chimas described herein.

GMCSF is associated with growth and differentiation of hematopoietic cells and is a potent immunostimulator with pleiotropic effects (e.g., augmentation of antigen presentation in numerous cells). Additionally, it is involved in increased expression of MHC II on monocytes and adhesion molecules on granulocytes and monocytes. Furthermore, GMCSF is involved in the amplification of T cell proliferation. In certain embodiments of the current invention, GMCSF comprises the immunostimulant domain in the antibody-immunostimulant fusions used in the invention.

In certain embodiments of the invention, the immunostimulant domain(s) comprises a chemokine (or a fragment thereof). In certain embodiments chemokines selectively attract various leukocytes to specific locations and can induce not only cell migration but also activation. Chemokines are typically classified into alpha, beta, and gamma sub-types. Their classification is divided according to the configuration of the first cysteine residues at the amino terminus of the protein. Different classifications of chemokines act to attract different classes of inflammatory cells. Thus, use of such different chemokines in the chimeric moieties of the present invention can result in different immune responses activated in a subject that is treated with such moieties. Chemokines used in the chimeric moieties of this invention include, but are not limited to, C-X-C group chemokines, IL-8, mip2α, mip2β, PF4, platelet basic protein, hIP10, C-C family chemokines, LD78, Act-2, MCAF, 1309, RANTES, TCA3, IP-10, C chemokines, lymphotactin, CX3C (or c-x3-c) chemokines, fractalkine, etc.

Other embodiments of the invention comprise chimeric antibody-immunostimulant moieties comprising immunostimulants other than cytokines or chemokines. For example, various chimeric moieties of this invention can comprise KLH (keyhole limpet hemocyanin) or other such immunogenic compounds, as well as “super antigens” that cause direct stimulation of T cells and/or B cells without direct antigen presentation. Super antigens and compounds such as KLH (as well as their use, etc.) are well known by those in the art (see, e.g., Johnson, et al. (1992) 1992, p. 92-101, and Sekaly, R. (ed.) “Bacterial Superantigens” Seminars in Immunol. Vol. 5, 1993).

Examples of certain particularly useful immunostimulant domains (e.g., as are included in optional embodiments of the compositions herein) include, but are not limited to, e.g., cytokines, chemokines, interleukins, interferons, C-X-C chemokines, C-C family chemokines, C chemokines, CX3C chemokines, super antigens, growth factors, IL-1, IL-2, IL-4, IL-6, IL-7, IL-8, IL-10, IL-12, IL-13, IL-17, IL-18, RANTES, mip1α, mip1β, GMCSF, GCSF, gamma interferon, alpha interferon, TNF, CSFs, mip2α, mip2β, PF4, platelet basic protein, hIP10, LD78, Act-2, MCAF, 1309, TCA3, IP-10, lymphotactin, fractalkine, KLH, and fragments (preferably of sufficient length to retain immunostimulatory activity) thereof of any of the above.

The actual specific immunostimulant molecule in various embodiments of the fusion proteins used in the invention (whether comprising a cytokine, chemokine, etc.) will depend upon, e.g., the specific disease state/condition, the specific antigen targeted, the specific action desired (e.g., elicitation of a humoral immune response, a cellular immune response, or both), etc.

C) Construction of Chimeric Moieties.

The antibody (antibodies) and the immunostimulatory molecule(s) comprising the chimeric moieties of this invention can typically be joined together in any order. Thus, for example, in mon-valent constructs where the antibody is a single chain antibody, the immunostimulatory molecule/domain can be attached to either the amino or carboxy termini of the antibody and/or to an internal region of the antibody. Similarly, in certain embodiments, the antibody can be attached to an internal location or a terminus of one or more immunostimulatory molecule(s). In any case, attachment points are selected that do not interfere with the respective activities of antibody and/or immunostimulatory molecule(s)/domain(s).

The antibody and the immunostimulatoyr component(s) can be attached by any of a number of means well known to those of skill in the art. In certain embodiments immunostimulatory component(s) are conjugated, either directly or through a linker (spacer), to the antibody. However, where both the immunostimulatory component(s) and the antibody are both polypeptides it is preferable to recombinantly express the chimeric molecule as a fusion protein.

1) Conjugation of the Immunostimulatory Components to the Antibody.

In one embodiment, the antibody is chemically conjugated to one or both immunostimulatory component(s). Means of chemically conjugating molecules are well known to those of skill.

The procedure for attaching an immunostimulatory molecule to an antibody will vary according to the chemical structure of the agent. Polypeptides typically contain variety of functional groups; e.g., carboxylic acid (COOH) or free amine (—NH₂) groups, that are available for reaction with a suitable functional group on an effector molecule to bind the effector thereto.

Alternatively, the antibody and/or immunostimulatory component(s) can be derivatized to expose or attach additional reactive functional groups. The derivatization can involve attachment of any of a number of linker molecules such as those available from Pierce Chemical Company, Rockford Ill.

A “linker”, as used herein, is a molecule that is used to join the antibody to the immunostimulatory component(s) comprising the chimeric moiety. The linker is typically capable of forming covalent bonds to both the antibody and to the immunostimulatory moiety. Suitable linkers are well known to those of skill in the art and include, but are not limited to, straight or branched-chain carbon linkers, heterocyclic carbon linkers, or peptide linkers. Where the antibody and the immunostimulatory molecule are polypeptides, the linkers can be joined to the constituent amino acids through their side groups (e.g., through a disulfide linkage to cysteine). However, in certain preferred embodiment, the linkers will be joined to the alpha carbon amino and carboxyl groups of the terminal amino acids.

A bifunctional linker or trifunctional linker having one functional group reactive with a group on each component of the chimeric moiety, can be used to form the desired immunoconjugate. Alternatively, in certain embodiments derivatization can involve chemical treatment of the antibody, e.g., glycol cleavage of a sugar moiety of a glycoprotein antibody with periodate to generate free aldehyde groups. The free aldehyde groups on the antibody can be reacted with free amine or hydrazine groups on, e.g., a linker bind the polypeptide (see, e.g., U.S. Pat. No. 4,671,958). Procedures for generation of free sulfhydryl groups on polypeptide, such as antibodies or antibody fragments, are also known (see, e.g., U.S. Pat. No. 4,659,839).

Many procedures and linker molecules for attachment of various compounds to proteins such as antibodies are known (see, e.g., European Patent Application No. 188,256; U.S. Pat. Nos. 4,671,958, 4,659,839, 4,414,148, 4,699,784; 4,680,338; 4,569,789; and 4,589,071; and Borlinghaus et al. (1987) Cancer Res. 47: 4071-4075).

2) Production of Fusion Proteins.

Where the antibody and/or the immunostimulatory component of the chimeric moiety are both single chain proteins and relatively short (i.e., less than about 50 amino acids) they can be synthesized using standard chemical peptide synthesis techniques. Where both componets are relatively short the chimeric moiety can be synthesized as a single contiguous polypeptide. Alternatively the antibody and the effector molecule may be synthesized separately and then fused by condensation of the amino terminus of one molecule with the carboxyl terminus of the other molecule thereby forming a peptide bond. Alternatively, the antibody and immunostimulatory molecule(s) may each be condensed with one end of a peptide spacer molecule thereby forming a contiguous fusion protein.

Solid phase synthesis in which the C-terminal amino acid of the sequence is attached to an insoluble support followed by sequential addition of the remaining amino acids in the sequence is the preferred method for the chemical synthesis of the polypeptides of this invention. Techniques for solid phase synthesis are described by Barany and Merrifield, Solid-Phase Peptide Synthesis; pp. 3-284 in The Peptides: Analysis, Synthesis, Biology. Vol. 2: Special Methods in Peptide Synthesis, Part A., Merrifield, et al. J. Am. Chem. Soc., 85: 2149-2156 (1963), and Stewart et al., Solid Phase Peptide Synthesis, 2nd ed. Pierce Chem. Co., Rockford, Ill. (1984).

In certain embodiments, chimeric fusion proteins of the present invention are synthesized using recombinant DNA methodology. Generally this involves creating a DNA sequence that encodes the fusion protein, placing the DNA in an expression cassette under the control of a particular promoter, expressing the protein in a host, isolating the expressed protein and, if required, renaturing the protein.

DNA encoding the fusion proteins of this invention can be prepared by any suitable method, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066.

Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences can be obtained by the ligation of shorter sequences.

Alternatively, subsequences can be cloned and the appropriate subsequences cleaved using appropriate restriction enzymes. The fragments can then be ligated to produce the desired DNA sequence.

Thus, for example DNA encoding fusion proteins of the present invention can be cloned using DNA amplification methods such as polymerase chain reaction (PCR). Thus, for example, the nucleic acid encoding a particular antibody is PCR amplified, using a sense primer containing the restriction site for NdeI and an antisense primer containing the restriction site for HindIII. This produces a nucleic acid encoding the antibody sequence and having terminal restriction sites. A nucleic acid encoding the immunostimulatory molecule(s) can similarly be produces. Ligation of the antibody and immunostimulatory molecule sequences and insertion into a vector produces a vector encoding the antibody joined to the immunostimulatory molecule.

While the components comprising the chimeric moiety can be joined directly together, in certain embodiments, the components can be separated by a peptide spacer consisting of one or more amino acids. Generally the spacer will have no specific biological activity other than to join the proteins or to preserve some minimum distance or other spatial relationship between them. However, the constituent amino acids of the spacer can be selected to influence some property of the molecule such as the folding, net charge, or hydrophobicity.

The nucleic acid sequences encoding the fusion proteins can be expressed in a variety of host cells, including E. coli, other bacterial hosts, yeast, and various higher eukaryotic cells such as the COS, CHO and HeLa cells lines and myeloma cell lines. The recombinant protein gene will be operably linked to appropriate expression control sequences for each host. For E. coli this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

The plasmids of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant fusion proteins can be purified according to standard procedures of the art, including ammonium sulfate precipitation, affinity columns, column chromatography, gel electrophoresis and the like (see, generally, R. Scopes (1982) Protein Purification, Springer-Verlag, N.Y.; Deutscher (1990) Methods in Enzymology Vol. 182: Guide to Protein Purification., Academic Press, Inc. N.Y.). Substantially pure compositions of at least about 90 to 95% homogeneity are preferred, and 98 to 99% or more homogeneity are most preferred for pharmaceutical uses. Once purified, partially or to homogeneity as desired, the polypeptides may then be used therapeutically.

One of skill in the art would recognize that after chemical synthesis, biological expression, or purification, the fusion protein can possess a conformation substantially different than the native conformations of the constituent polypeptides. In this case, it may be necessary to denature and reduce the polypeptide and then to cause the polypeptide to re-fold into the preferred conformation. Methods of reducing and denaturing proteins and inducing re-folding are well known to those of skill in the art (See, Debinski et al. (1993) J. Biol. Chem., 268: 14065-14070; Kreitman and Pastan (1993) Bioconjug. Chem., 4: 581-585; and Buchner, et al. (1992) Anal. Biochem., 205: 263-270).

One of skill would recognize that modifications can be made to the fusion proteins without diminishing their biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids placed on either terminus to create conveniently located restriction sites or termination codons.

The construction of illustrative chimeric fusion proteins of the present invention is illustrated in detail in the Examples.

D) Chimeric Moiety and Antigen Complexes.

In certain embodiments the antibody-immunostimulant fusion proteins and specific disease related antigen are incubated together for selected (or specific) periods of time (e.g., in order for immunocomplexes to form between the antigen and the fusion protein) before the composition is administered to the subject. In various embodiments such incubations are done at 4° C., e.g. overnight. In various embodiments the incubation temperatures and times vary. The determination of incubation conditions, and/or times, and/or temperatures can be determined based upon, e.g., the specific antigen involved, the specific chimeric moieties involved, the affinity between the antigen and the antibodies, etc.

Where the chimeric moiety is incubated with the antigen, the ratio of the number of molecules of chimeric moiety to the number of molecules of antigen can be roughtly 1:1. In some embodiments, however, the chimeric moiety exceeds the number of antigen molecules, while in certain other embodiments, the antigen molecules exceed the molecules of chimeric moiety (e.g., in certain embodiments, the number of antigen molecules is great enough to essentially saturate the chimeric moieties thereby forming complexes of essentially all the chimeric moieties). In some typical embodiments, the amount of each component is allocated so that the binding unit equivalents of each component are equal or roughly/approximately equal (see, e.g., Example I).

In some embodiments, the various constituents of the compositions come pre-measured and/or prepackaged and/or ready for use without additional measurement, etc. The present invention also optionally comprises kits for conducting/using the methods and/or the compositions of the invention. In particular, these kits optionally include, e.g., appropriate antibody-immunostimulant fusion proteins (and optionally mixtures of a number of such proteins for performing synergistic treatments, see, above), and optionally appropriate disease related antigen(s) as well). Additionally, such kits can also comprise appropriate excipients (e.g., pharmaceutically acceptable excipients) for performing therapeutic and/or prophylactic treatments of the invention. Such kits optionally contain additional components for the assembly and/or use of the compositions of the invention including, but not limited to, e.g., diluents, adjuvants, etc.

III. Administration of Antibody-Immunostimulants as Adjuvants of Protein Vaccination

A) Pharmaceutical Formulations.

In certain embodiments the chimeric moieties of this invention are administered to subjects in need of treatment (either therapeutically or prophylactically). In certain embodiments the chimeric moieties are administered in conjunction with antigens specifically bound by the chimeric moieties. In certain embodiments the chimeric moieties are provided as a complex with the antigen.

Typically the chimeric moiety and/or antigen are provided in an appropriate sterile pharmaceutical carrier. Such pharmaceutical carriers can act to maintain the solubility and in certain embodiments the activity of the chimeric moieties and, when present, the antigens. In some embodiments, it may be desired to administer additional components in conjunction with the fusion proteins and/or antigens. For example, in some treatment regimes, chemotherapeutic agents, antibiotics, additional antibody fusion proteins comprising growth factors, etc. are all optionally included with the compositions of the invention.

In order to carry out the methods of the invention, one or more active agents (e.g., chimeric moieties and/or antigens) of this invention are administered, e.g. to an individual diagnosed as having one or more symptoms of cancer or other pathology (e.g., bacterial infection, fungal infection, etc., e.g., as described herein), or as being at risk for cancer or other pathologies described hereien. The active agent(s) can be administered in the “native” form or, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

For example, acid addition salts are prepared from the free base using conventional methodology, that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are halide salts, such as may be prepared using hydrochloric or hydrobromic acids. Conversely, preparation of basic salts of the active agents of this inventioni are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

Preparation of esters typically involves functionalization of hydroxyl and/or carboxyl groups which may be present within the molecular structure of the drug. The esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides and prodrugs can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety that results in a compound that is therapeutically inactive until modified by an individual's metabolic system.

B) Administration.

The active agents identified herein are useful for parenteral, topical, oral, nasal (or otherwise inhaled), rectal, or local administration, such as by aerosol or transdermally, for prophylactic and/or therapeutic treatment of one or more of the pathologies/indications described herein (e.g., atherosclerosis and/or symptoms thereof). The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, lipid complexes, etc.

The active agents of this invention are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

The excipients are preferably sterile and generally free of undesirable matter. These compositions may be sterilized by conventional, well-known sterilization techniques.

In therapeutic applications, the compositions of this invention are administered to a patient suffering from one or more symptoms of the one or more pathologies described herein, or at risk for one or more of the pathologies described herein in an amount sufficient to prevent and/or cure and/or or at least partially prevent or arrest the disease and/or its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

The concentration of active agent(s) can vary widely, and will be selected primarily based on fluid volumes, viscosities, body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic regimen in a particular subject or group of subjects.

In certain preferred embodiments, the active agents of this invention are administered orally (e.g. via a tablet) or as an injectable in accordance with standard methods well known to those of skill in the art. In other preferred embodiments, the active agents, can also be delivered through the skin using conventional transdermal drug delivery systems, i.e., transdermal “patches” where the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Other preferred formulations for topical drug delivery include, but are not limited to, ointments and creams. Ointments are semisolid preparations which are typically based on petrolatum or other petroleum derivatives. Creams containing the selected active agent, are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

In certain embodiments, peptide delivery can be enhanced by the use of protective excipients. This is typically accomplished either by complexing the polypeptide with a composition to render it resistant to acidic and enzymatic hydrolysis or by packaging the polypeptide in an appropriately resistant carrier such as a liposome. Means of protecting polypeptides for oral delivery are well known in the art (see, e.g., U.S. Pat. No. 5,391,377 describing lipid compositions for oral delivery of therapeutic agents).

Elevated serum half-life can be maintained by the use of sustained-release protein “packaging” systems. Such sustained release systems are well known to those of skill in the art. In one preferred embodiment, the ProLease biodegradable microsphere delivery system for proteins and peptides (Tracy (1998) Biotechnol. Prog. 14: 108; Johnson et al. (1996), Nature Med. 2: 795; Herbert et al. (1998), Pharmaceut. Res. 15, 357) a dry powder composed of biodegradable polymeric microspheres containing the active agent in a polymer matrix that can be compounded as a dry formulation with or without other agents.

The ProLease microsphere fabrication process was specifically designed to achieve a high encapsulation efficiency while maintaining integrity of the active agent. The process consists of (i) preparation of freeze-dried drug particles from bulk by spray freeze-drying the drug solution with stabilizing excipients, (ii) preparation of a drug-polymer suspension followed by sonication or homogenization to reduce the drug particle size, (iii) production of frozen drug-polymer microspheres by atomization into liquid nitrogen, (iv) extraction of the polymer solvent with ethanol, and (v) filtration and vacuum drying to produce the final dry-powder product. The resulting powder contains the solid form of the active agents, which is homogeneously and rigidly dispersed within porous polymer particles. The polymer most commonly used in the process, poly(lactide-co-glycolide) (PLG), is both biocompatible and biodegradable.

Encapsulation can be achieved at low temperatures (e.g., −40° C.). During encapsulation, the protein is maintained in the solid state in the absence of water, thus minimizing water-induced conformational mobility of the protein, preventing protein degradation reactions that include water as a reactant, and avoiding organic-aqueous interfaces where proteins may undergo denaturation. A preferred process uses solvents in which most proteins are insoluble, thus yielding high encapsulation efficiencies (e.g., greater than 95%).

In another embodiment, one or more components of the solution can be provided as a “concentrate”, e.g., in a storage container (e.g., in a premeasured volume) ready for dilution, or in a soluble capsule ready for addition to a volume of water.

The present invention includes methods of therapeutically or prophylactically treating a disease or disorder, eliciting an immune response (humoral and/or cellular) in a subject and administering an immunological composition by administering in vivo or ex vivo one or more nucleic acids or polypeptides/fusion proteins/antigens or conjugates of the invention as described herein (or compositions comprising a pharmaceutically acceptable excipient and one or more such nucleic acids or polypeptides and/or fusion proteins and/or antigens) to a subject, including, e.g., a mammal, including, e.g., a human, primate, mouse, pig, cow, goat, rabbit, rat, guinea pig, hamster, horse, sheep; or a non-mammalian vertebrate such as a bird (e.g., a chicken or duck) or a fish, or invertebrate.

In certain embodiments of the invention utilizing ex vivo methods, one or more cells or a population of cells of interest of the subject (e.g., dendritic cells, antigen presenting cells, etc.) are obtained or removed from the subject and contacted with an amount of a fusion protein and antigen of the invention that is effective in prophylactically or therapeutically treating a disease, disorder, or other condition. The contacted cells are then returned or delivered to the subject to the site from which they were obtained or to another site (e.g., via intramuscular injection, etc.) of interest in the subject to be treated. The methods/compositions of the invention optionally elicit an effective immune response whether such cells are delivered to a site of need (e.g., a tumor or infection site) or to a site unrelated to such (e.g., a distant body part, etc.). If desired, the contacted cells may be deposited, injected, grafted, etc. onto a tissue, organ, or system site (including, e.g., tumor cells, tumor tissue sample, organ cells, blood cells, cells of the skin, lung, heart, muscle, brain, mucosae, liver, intestine, spleen, stomach, lymphatic system, cervix, vagina, prostate, mouth, tongue, etc) of interest in the subject using standard and well-known depositing, injection and grafting techniques or, e.g., delivered to the blood or lymph system using standard delivery or transfusion techniques.

In certain embodiments this invention provides in vivo methods in which one or more cells or a population of cells of interest of the subject are contacted directly or indirectly with an amount of a chimeric moiety, antibody fusion protein, and/or antigen of the invention effective in prophylactically or therapeutically treating a disease, disorder, or other condition. In either format, the antibody fusion protein and/or antigen is optionally administered or transferred to the cells (e.g., tumor cells, tumor tissue sample, infection site (such as an abscess, etc.) organ cells, blood cells, cells of the skin, lung, heart, muscle, brain, mucosae, liver, intestine, spleen, stomach, lymphatic system, cervix, vagina, prostate, mouth, tongue, etc.) by any of a variety of formats, including topical administration, injection (e.g., by using a needle or syringe), or vaccine or gene gun delivery, pushing into a tissue, organ, or skin site. The molecules can be delivered, for example, intramuscularly, intradermally, subdermally, subcutaneously, orally, intraperitoneally, intrathecally, intravenously, or placed within a cavity of the body (including, e.g., during surgery), or by inhalation or vaginal or rectal administration. In more typical embodiments, the antibody fusion protein and/or antigen of the invention are optionally administered or transferred to a site that is not directly in need of treatment, etc. For example, in typical embodiments, the antibody fusion protein and/or antigen of the invention are injected (e.g., see, above), e.g., intramuscularly or intravenously at a site distant from, e.g. a tumor, infection site, etc. (e.g., injection into the flank of an animal when the tumors to be combated are in the lungs, etc.). The immune response is still generated by the antibody-immunostimulant fusion proteins/antigen compositions of the invention.

In certain embodiments, the invention provides ex vivo methods in which one or more cells of interest or a population of cells of interest of the subject (e.g., tumor cells, tumor tissue sample, organ cells, blood cells, cells of the skin, lung, heart, muscle, brain, mucosae, liver, intestine, spleen, stomach, lymphatic system, cervix, vagina, prostate, mouth, tongue, etc.) are obtained or removed from the subject and transformed by contacting said one or more cells or population of cells with a polynucleotide construct comprising a target nucleic acid sequence encoding antibody-immunostimulant fusion proteins and/or antigen used in the invention, as biologically active molecules that are effective in prophylactically or therapeutically treating the disease, disorder, or other condition. The one or more cells or population of cells is contacted with a sufficient amount of the polynucleotide construct (e.g., encoding antibody-immunostimulant fusion proteins and/or antigen) and a promoter controlling expression of said nucleic acid sequence such that uptake of the polynucleotide construct (and promoter) into the cell(s) occurs and sufficient expression of the target nucleic acid sequence of the invention results to produce an amount of the biologically active molecules effective to prophylactically or therapeutically treat the disease, disorder, or condition. The polynucleotide construct may include a promoter sequence (e.g., CMV promoter sequence) that controls expression of the nucleic acid sequence of the invention and/or, if desired, one or more additional nucleotide sequences encoding at least one or more of another molecule of the invention, such as a cytokine, adjuvant, or co-stimulatory molecule, or other polypeptide, etc. of interest, etc.

Following transfection, the transformed cells optionally are returned, delivered, or transferred to the subject to the tissue site or system from which they were obtained or to another site (e.g., tumor cells, tumor tissue sample, organ cells, blood cells, cells of the skin, lung, heart, muscle, brain, mucosae, liver, intestine, spleen, stomach, lymphatic system, cervix, vagina, prostate, mouth, tongue, etc.) in the subject. If desired, the cells may be grafted onto a tissue, skin, organ, or body system of interest in the subject using standard and well-known grafting techniques or delivered to the blood or lymphatic system using standard delivery or transfusion techniques. Such delivery, administration, or transfer of transformed cells is typically made by using one or more of the routes or modes of administration described above. Expression of the target nucleic acid occurs naturally or can be induced and an amount of the encoded antibody-immunostimulant fusion proteins and/or antigen is expressed sufficient and effective to treat the disease or condition. The site of expression of the compositions, etc. need not be at or near the site of need in the subject. As explained throughout, the antibody-immunostimulant fusion proteins and/or antigens in the compositions of the invention do not necessarily need to come into direct contact with, e.g., a tumor cell, infectious organism, etc. in order to elicit an immune response against such, e.g., tumor or infection.

In various embodiments the invention provides in vivo methods in which one or more cells of interest or a population of cells of the subject (e.g., including those cells and cells systems and subjects described above) are transformed in the body of the subject by contacting the cell(s) or population of cells with (or administering or transferring to the cell(s) or population of cells using one or more of the routes or modes of administration described above) a polynucleotide construct comprising a nucleic acid sequence that encodes a biologically active antibody-immunostimulant fusion protein and/or antigen used in the invention that is effective in prophylactically or therapeutically treating the disease, disorder, or other condition.

The polynucleotide construct optionally can be administered or transferred to cell(s) by first directly contacting cells using one or more of the routes or modes of administration described above with a sufficient amount of the polynucleotide construct comprising the nucleic acid sequence encoding the biologically active molecules, and a promoter controlling expression of the nucleic acid sequence, such that uptake of the polynucleotide construct (and promoter) into the cell(s) occurs and sufficient expression of the nucleic acid sequence of the invention results to produce an amount of the biologically active antibody fusion protein and/or antigen effective to prophylactically or therapeutically treat the disease or disorder. Expression of the target nucleic acid occurs naturally or can be induced such that an amount of the encoded antibody fusion protein and/or antigen is expressed sufficient and effective to treat the disease or condition by eliciting the appropriate immune response. The polynucleotide construct may include a promoter sequence (e.g., CMV promoter sequence) that controls expression of the nucleic acid sequence and/or, if desired, one or more additional nucleotide sequences encoding at least one or more of another molecule used in the invention, a cytokine, adjuvant, or co-stimulatory molecule, or other such molecules of interest.

In each of the in vivo and ex vivo treatment methods as described above, a composition comprising an excipient and the antibody fusion protein and/or antigen or nucleic acid encoding such as used in the invention can be administered or delivered. In one aspect, a composition comprising a pharmaceutically acceptable excipient and such molecules or nucleic acid as used in the invention is administered or delivered to the subject as described above in an amount effective to treat the disease or disorder.

In another aspect, in each in vivo and ex vivo treatment method described above, the amount of polynucleotide administered to the cell(s) or subject can be an amount sufficient that uptake of said polynucleotide into one or more cells of the subject occurs and sufficient expression of said nucleic acid sequence results to produce an amount of the biologically active molecules effective to enhance or elicit an immune response in the subject. In another aspect, for each such method, the amount of molecules administered to cell(s) or subject can be an amount sufficient to enhance or elicit an immune response in the subject.

In yet another aspect, in an in vivo or ex vivo treatment method in which a polynucleotide construct (or composition comprising a polynucleotide construct) is used, the expression of the polynucleotide construct can be induced by using an inducible on-and-off gene expression system. Examples of such on-and-off gene expression systems include the Tet-On™ Gene Expression System and Tet-Off™ Gene Expression System, respectively. Other controllable or inducible on-and-off gene expression systems are known to those of ordinary skill in the art. With such system, expression of the target nucleic of the polynucleotide construct can be regulated in a precise, reversible, and quantitative manner. Gene expression of the target nucleic acid can be induced, for example, after the stable transfected cells containing the polynucleotide construct comprising the target nucleic acid are delivered or transferred to or make contact with a tissue site, organ or system of interest. Such systems are of particular benefit in treatment methods and formats in which it is advantageous to delay or precisely control expression of the target nucleic acid (e.g., to allow time for completion of surgery and/or healing following surgery; to allow time for the polynucleotide construct comprising the target nucleic acid to reach the site, cells, system, or tissue for expression; to allow time for the graft containing cells transformed with the construct to become incorporated into the tissue or organ onto or into which it has been spliced or attached, etc.).

In some embodiments, the multiple compositions are used to treat the subject. For example, multiple dosages of the fusion protein/antigen mixture are optionally given to a subject over a prescribed time period. Ranges for such are optionally highly variable depending upon, e.g., the subject's response to treatment, any toxicities and/or or adverse reactions to treatment, etc. and are optionally adjusted to suit each individual treatment regime/subject. Additionally, the fusion protein is optionally given to the subject in a separate composition than the antigen mixture. For example, the antigen composition is optionally administered to the subject prior to, approximately concurrently to, or after the fusion protein composition is administered to the subject. Furthermore, as mentioned herein, some disease states/conditions present situations where a separate administration of disease related antigen is not given. For example, some HER2/neu expressing tumors shed large amounts of the HER2/neu antigen. In optional embodiments, the current invention utilizes such shed antigen by optionally using such to form immunocomplexes with the fusion proteins administered. Again, such optional mechanism of action should not be construed as limiting upon the efficaciousness of the methods and compositions of the current invention.

The foregoing formulations and administration methods are intended to be illustrative and not limiting. It will be appreciated that, using the teaching provided herein, other suitable formulations and modes of administration can be readily devised.

IV. Kits.

The compositions described herein are optionally packaged to include all (or certain) necessary components for performing the methods of the invention or for using the compositions of the invention (optionally including, e.g., written instructions for the use of the methods/compositions of the invention). For example, the kits can optionally include such components as, e.g., buffers, reagents, serum proteins, antibodies, substrates, etc. In the case of prepackaged reagents, the kits optionally include pre-measured or pre-dosed amounts that are ready to incorporate into the methods without measurement, e.g., pre-measured fluid aliquots, or pre-weighed or pre-measured solid reagents that can be easily reconstituted by the end-user of the kit.

Such kits also typically include appropriate instructions for performing the methods of the invention and/or using the compositions of the invention. In certain embodiments such instructional materials can provide recommended dosages, counterindications, drug incompatibilities, and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

In some embodiments, the components of the kits/packages are provided in a stabilized form, so as to prevent degradation or other loss during prolonged storage, e.g., from leakage. A number of stabilizing processes/agents are widely used for reagents, etc. that are to be stored, such as the inclusion of chemical stabilizers (i.e., enzymatic inhibitors, microbicides/bacteriostats, anticoagulants), etc.

### EXAMPLES

The following examples are offered to illustrate, but not to limit the claimed invention.

### Example I

### Anti-HER2/neu Antibody Fusion Proteins as Effective Enhancers of Extracellular Domain HER2/neu Protein Vaccination

The molecule HER2/neu is overexpressed in a number of human cancers (e.g., breast, ovarian, prostate and lung cancers) and is associated with poor prognosis. As described above, some DNA and peptide based vaccines which target HER2/neu have elicited significant protection against HER2/neu expressing cancers in animal models. However, vaccines using the complete extracellular domain of HER2/neu (ECD^{HER2}) have not shown the same efficacy. As detailed herein, the current invention illustrates several anti-human HER2/neu antibody (Ab)-immunostimulant fusion proteins which contain the immunostimulatory cytokines: IL-2, IL-12 or GMCSF and their use (again, depending upon, e.g., the specific disease to be treated, the specific action to be potentiated, etc. different immunostimulatory molecules are optionally fused to construct the molecules used in the current invention).

The antibody-immunostimulant fusion proteins used in Example I (and also similar related fusions of the invention) retain both immunostimulant (e.g., cytokine) activity and the ability to bind HER2/neu. To determine if these antibody-immunostimulant fusion proteins act as immunoenhancers for ECD^{HER2} vaccination, mice were vaccinated with either human ECD^{HER2}, ECD^{HER2} with anti-HER2/neu antibody (IgG3), or ECD^{HER2} with each antibody-immunostimulant fusion protein. After given a booster, mice were challenged with a syngeneic carcinoma that expressed the rat HER2/neu protein (i.e., TUBO). There was a significant retardation of tumor growth rate as well as in increase in long-term survivors in the groups of mice vaccinated with ECD^{HER2} plus all three antibody-immunostimulant fusion proteins as compared to the mice in the control groups (i.e., those mice given PBS, ECD^{HER2} or ECD^{HER2} plus IgG3).

An anti-ECD^{HER2} humoral immune response was detected in all vaccinated groups, with ECD^{HER2} plus IgG3-(GMCSF) and ECD^{HER2} plus IgG3-(IL-2) vaccinated mice showing enhanced levels. These two groups had increased level of anti-ECD^{HER2} IgG1 and IgG2a antibodies, as compared to the control groups. These results indicate that both T_{H}2 and T_{H}1 immune responses were elicited. The mice vaccinated with ECD^{HER2} plus IgG3-(IL-12) showed increased IgG2a antibodies but not IgG1 antibodies, indicating that a T_{H}1 immune responses was elicited (see, above).

Immune sera from the mice showed significant in vitro anti-proliferative activity against SK-BR-3 (a human breast cancer with overexpressed HER2/neu protein), with the level of inhibition correlated with the level of anti-ECD^{HER2} antibody. When incubated with soluble ECD^{HER2}, splenocytes from mice vaccinated with ECD^{HER2} plus antibody-(GMCSF) demonstrated significant proliferation and significant IFN-γ secretion as compared with the other groups. Such results indicate that the current invention (as illustrated by the example) elicits both humoral and cell-mediated responses. Thus, both the humoral and the cell-mediated immune responses can contribute to the observed anti-tumor activity (as seen in the current example). The current examples indicate that, through use of the current invention, it is possible to use anti-HER2/neu antibody-immunostimulant fusion proteins as adjuvants of protein vaccination as prophylactic and therapeutic regimens against, e.g., HER2/neu expressing tumors in patients. Importantly, patients who are unresponsive to other anti-HER2/neu antibody based treatments can optionally benefit through use of the methods and compositions of the current invention (as illustrated in the current example). Once again, it is important to emphasize that other combinations of antibodies/immunostimulants/antigens can be targeted against different diseases, and are included in the current invention.

In the current example illustrating the invention, female BALB/c mice were vaccinated with the human ECD^{HER2} protein in various compositions. Vaccinated mice were challenged with a transplantable carcinoma, TUBO, which overexpresses the rat neu protein. See, e.g., Rovero, S. A. et al. 2000, “DNA vaccination against rat her-2/Neu p185 more effectively inhibits carcinogenesis than transplantable carcinomas in transgenic BALB/c mice” J Immunol 165:5133. As seen below, mice immunized only with soluble ECD^{HER2} showed only modest anti-tumor immunity compared to the control group. However, when the immuno-enhancing cytokines IL-2, IL-12 or GMCSF were fused to an anti-HER2/neu antibody (human IgG3) and used as vaccine adjuvants (i.e., as per the methods of the current invention), a remarkable enhancement of anti-tumor activity was seen. See, e.g., Peng, L. S., et al. 1999 “A single-chain IL-12 IgG3 antibody fusion protein retains antibody specificity and IL-12 bioactivity and demonstrates antitumor activity” J Immunol 163:250; Penichet, M. L., et al. 2001 “A recombinant IgG3-(IL-2) fusion protein for the treatment of human HER2/neu expressing tumors” Hum Antibodies 10:43; and Dela Cruz, J. S., et al. 2000 “Recombinant anti-human HER2/neu IgG3-(GMCSF) fusion protein retains antigen specificity and cytokine function and demonstrates antitumor activity” J Immunol 165:5112 for further information on anti-HER2 fusion proteins all of which are incorporated herein for all purposes.

Materials and Method

Mice

In the current example, female BALB/c mice 10-12 weeks of age obtained from Taconic Farms, Inc. (Germantown, N.Y.) were used. All experiments (both in Example I and Example II) were performed according to National Institutes of Health (NIH) (Bethesda, Md.) Guide for the Care and Use of Laboratory Animals.

Cell Lines

TUBO is a cloned cell line, which overexpresses the neu protein. The cell line was established from a lobular carcinoma that spontaneously arose in a BALB/c female mouse transgenic for the transforming rat neu oncogene driven by the mouse mammary tumor virus promoter (see, Rovero et al, supra). TUBO cells grow progressively in normal BALB/c mice and give rise to lobular carcinomas which are histologically similar to those carcinomas that appear in BALB-neuT-transgenic mice, again, see, Rover, S., supra. In the current example, TUBO cells were cultured in Dubecco's Modified Eagle Medium (DMEM) supplemented with glutamax, glucose, 25 mM Hepes buffer, pyridoxine-HCl (GibcoBRL, Life Technologies, Rockville, Md.), and 20% fetal bovine serum (Atlas Biologicals, Fort Collins, Colo.). Also used in the current example was SK-BR-3, a human breast cancer cell line which overexpresses the HER2/neu protein (ATCC, Rockville, Md.). SK-BR-3 cells were cultured in Iscoves Modified Dubecco's Medium, IMDM, supplemented with L-glutamine, penicillin, and streptomycin with 5% bovine calf serum (Atlanta Biologicals, Norcross, Ga.).

Antibody-Immunostimulant Fusion Proteins and ECD^{HER2}

The construction, purification and analysis of biological activities of IgG3, IgG3-(GMCSF), IgG3-(IL-2) and IgG3-(IL-12) immunostimulant fusion proteins was described previously. See, Peng, L. S., et al. 1999 “A single-chain IL-12 IgG3 antibody fusion protein retains antibody specificity and IL-12 bioactivity and demonstrates antitumor activity” J Immunol 163:250; Penichet, M. L., et al. 2001 “A recombinant IgG3-(IL-2) fusion protein for the treatment of human HER2/neu expressing tumors” Hum Antibodies 10:43; and Dela Cruz, J. S., et al. 2000 “Recombinant anti-human HER2/neu IgG3-(GMCSF) fusion protein retains antigen specificity and cytokine function and demonstrates antitumor activity” J Immunol 165:5112 all of which are incorporated herein for all purposes. The IgG3 and antibody-immunostimulant fusion proteins used in this example contain the same variable region as the monoclonal anti-HER2/neu, Herceptin. BHK/erbB2, which is a cell line that secretes soluble human ECD^{HER2} was provided by Dr. James D. Marks (University of California at San Francisco, San Francisco, Calif.). The soluble ECD^{HER2} was purified from the BHK/erbB2 culture supernatants using affinity chromatography with IgG3 immobilized on Sepharose 4B (CNBr-activated Sepharose 4B, Amersham Pharmacia Biotech, Upsala, Sweden). All purified proteins were dialyzed against dialysis buffer (50 mM Tris base, 150 mM NaCl in deionized water at pH 7.8) and the concentrations were determined by bicinchoninic acid based protein assay (BCA protein Assay, Pierce Chemical Co., Rockford, Ill.). Prior to use, the proteins were analyzed by SDS-PAGE and Coomassie blue stained to assess purity, size and integrity.

Mice Vaccination and Challenge with TUBO

Two groups of eight mice were injected subcutaneously in their right flanks on day 0 and again on day 35 (week 5 “booster”) with either 8 μg of ECD^{HER2} alone, 8 μg of ECD^{HER2} plus 14 μg of IgG3, 8 μg of ECD^{HER2} plus 16 μg of IgG3-(GMCSF), 8 μg of ECD^{HER2} plus 16 μg of IgG3-(IL-2), or 8 μg of ECD^{HER2} plus 27 μg of IgG3-(IL-12). It will be appreciated that differing volumes of components were used in order to equalize the molarity to achieve 1:1 equivalence of binding units amongst the constituents of the composition. Antibody or antibody-immunostimulant fusion proteins were mixed with ECD^{HER2} to allow a 1 ECD^{HER2}: 1 F(ab′)₂ ratio, at a concentration which allowed the injection of 150 μl per mouse. The mixtures were allowed to sit at 4° C. overnight prior to injection. Mice injected with a diluent (PBS) served as a control group. Three weeks after the booster (i.e., three weeks after day 35), one set of vaccinated mice in each vaccination group was challenged in the left flank with 10⁶ TUBO cells in 150 μl Hank's balanced salt solution, HBSS (GIBCOBRL, Life Technologies, Rockville, Md.). One out of the eight mice vaccinated with ECD^{HER2} plus IgG3-(IL-2) died prior to a challenge with TUBO cells from a course unrelated to the vaccination.

Tumor growth in the mice was monitored and measured with a caliper beginning 7 days after the tumor challenge. Mice with tumors of 1.5 cm in diameter or greater were euthanized. On the same day the vaccinated mice were challenged with TUBO, blood (used in the serological studies and in passive transfer of immunity) and splenocytes were collected from the other group of unchallenged vaccinated mice and processed and used in additional studies described below.

Characterization of Murine Antibody Response to ECD^{HER2}

Sera obtained from mice 2 days prior to the challenge with TUBO or from the unchallenged vaccinated mice were analyzed by ELISA for antibodies to ECD^{HER2}. The ELISA was done using 96-well microtiter plates coated with 50 μl of ECD^{HER2} at a concentration of 1 μg/ml. The plates were washed and blocked with 3% bovine serum albumin (BSA) (Sigma Chemical, St. Louis, Mo.) in PBS. After washing, dilutions of sera in PBS containing 1% BSA were added to the wells and incubated overnight at 4° C. Bound IgG was detected by incubating for 1 hour at 37° C. with AP-labeled rabbit anti-mouse IgG (Zymed, San Francisco, Calif.). After washing, p-nitrophenyl phosphate disodium dissolved in diethanolamine buffer (Sigma Chemical, St. Louis, Mo.) was added for 2 hours and the plates were read at 410 nm. Sera from naïve mice of the same age were used as a negative control. All ELISAs were performed in duplicate using an internal positive control curve for each plate. Murine anti-ECD^{HER2} IgG1, IgG2a and IgG3 responses were analyzed by ELISA using 96-well microtiter plates prepared as described above with AP-labeled rat anti-mouse IgG1, IgG2a (Zymed, San Francisco, Calif.) or AP-labeled goat anti-mouse IgG3 (Southern Biotechnology Associates, Inc., Birmingham, Ala.) used as detecting agents.

TUBO and SK-BR-3 In Vitro Proliferation Assay

5×10³ TUBO cells or 2×10⁴ SK-BR-3 cells in 100 μl of IMDM (supplemented with L-glutamine, penicillin, streptomycin and 5% bovine calf serum) were added to each well of a 96-well round bottom tissue culture plate. Pooled sera from each regimen of cells were depleted of complement by incubation at 56° C. for 30 minutes and then diluted in IMDM supplemented with L-glutamine, penicillin, streptomycin and 5% bovine calf serum, to give a final working dilution of 1:100 and 1:300. Immune sera in a volume of 100 μl were added to the TUBO cells or the SK-BR-3 cells to give a final volume of 200 μl/well. These cells where then incubated for 48 hours or 6 days, (TUBO and SK-BR-3 respectively), in a 5% CO₂, 37° C. incubator. Twelve hours prior to the end of the incubation period, the wells of the plates were pulsed with ³H-thymidine (ICN, Costa Mesa, Calif.) to give a final concentration of 5 μCi/ml. The cells were then harvested and passed through a glass-fiber filter (Wallac Oy, Turku, Finland) using a Micro Cell Harvester (Skatron, Norway). Any ³H-thymidine incorporation into DNA by actively growing cells was measured with a 1205 Betaplate Liquid Scintillation Counter (Wallac Oy, Turku, Finland). All of the assays were done in triplicate.

It should be noted that the use of fewer cells and a shorter incubation period was required for the TUBO cell in vitro assay, because of the rapid growth of TUBO cells in culture as compared to SK-BR-3 cells. Data herein are presented as ³H-thymidine (CPM) incorporation by TUBO cells or by SK-BR-3 cells after incubation with immune sera. IgG3, containing the same variable region as Herceptin, is effective in inhibiting the growth of SK-BR-3 in vitro, and was used as a positive control. No positive control was available for use with TUBO cells.

Transfer of Immune Sera

The mice were randomized and distributed into groups of 6 mice per group. At day −1, naïve mice received an intravenous injection of 175 μl of pooled immune sera. On day 0, 10⁶ TUBO cells in 150 μl of HBSS (GIBCOBRL, Life Technologies, Rockville, Md.), were injected in the right flank of the mice. An untreated group of mice of the same age was also challenged with TUBO cells. Tumor growth was monitored and measured with a caliper starting at day 7 and every three days until day 21.

Mouse Splenocyte Isolation, Stimulation with Soluble ECD^{HER2} Protein and IFN-γ Quantification

Spleens from vaccinated mice were removed, pooled and teased with two frosted specimen slides using aseptic techniques. Released splenocytes were passed through a 100 μm cell strainer (Becton Dickinson Labware, Franklin Lanes, N.J.) to remove large debris. Red blood cells (RBCs) were lysed in 0.85% ammonium chloride in deionized water. 5×10⁶ splenocytes/ml/well were added into the wells of a 24-well tissue culture plate along with RPMI 1640 (GibcoBRL, Life Technologies, Rockville, Md.) supplemented with 50 IU/ml of murine IL-2 (PeproTech, Inc., Rocky Hill, N.J.), 10% fetal bovine serum and 1 μg/ml of soluble ECD^{HER2} protein. The well contents were incubated in a 5% CO₂, 37° C. incubator. After 84 hours, the wells were pulsed with 5 μCi of ³H-thymidine to a final concentration of approximately 5 μCi/ml for 12 hours (for a total stimulation period of 96 hours). Cells from a single well of the 24-well tissue culture plate were transferred to a 96-well round bottom tissue culture plate in quadruplicate and harvested. Any ³H-thymidine incorporation into DNA was measured as described above. Data herein are expressed as a stimulation index (SI) which is defined as the mean ³H CPM of the experimental wells divided by the mean ³H CPM of the control wells (splenocytes of mice vaccinated with PBS).

To determine the level of secreted IFN-γ, supernatants from a single well of a 24-well tissue culture plate were removed after 36 and 84 hours of stimulation and added, in duplicate, into 96-well microtiter plates that were pre-coated with an anti-IFN-γ capture antibody (PharMingen, San Diego, Calif.). The supernatants were diluted serially (1:2) and allowed to sit overnight at 4° C. The following day, the plates were washed and detecting AP-labeled antibody (PharMingen, San Diego, Calif.) was added. The plates were then allowed to sit at 37° C. for 1 hour. After washing, p-nitrophenyl phosphate disodium dissolved in diethanolamine buffer (Sigma Chemical, St. Louis, Mo.) was added to the wells and the plates were read at 410 nm. Quantitation of results was performed using a IFN-γ (PharMingen, San Diego, Calif.) standard curve generated in each plate. Data from such readings are presented as the concentration of IFN-γ (pg/ml) minus the background (PBS control) levels.

Statistical Analysis

All statistical analyses in the current example were made using the Mann-Whitney Rank Test, except for the survival curve for which the Trend Peto-Peto-Wilcoxon Test was used. For all cases, results were regarded as significant if the p values were <0.05.

Results:

ECD^{HER2} Vaccination and Anti-Tumor Activity

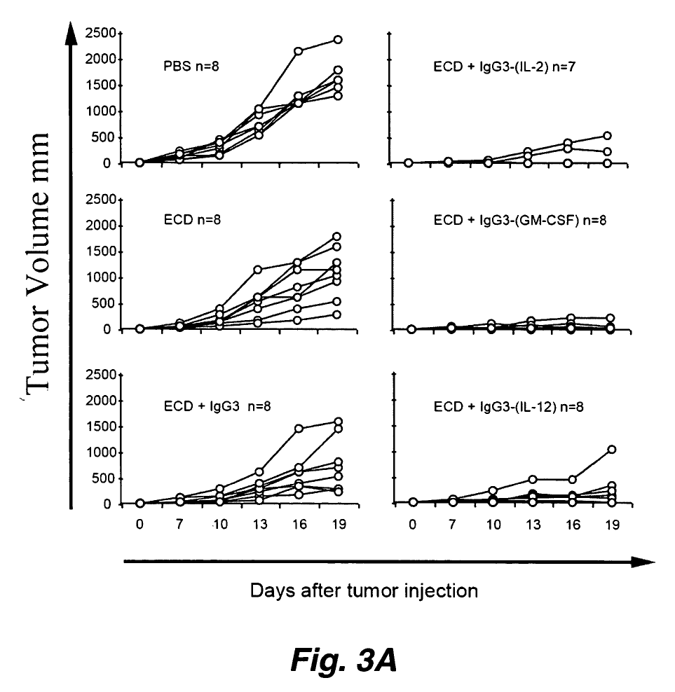

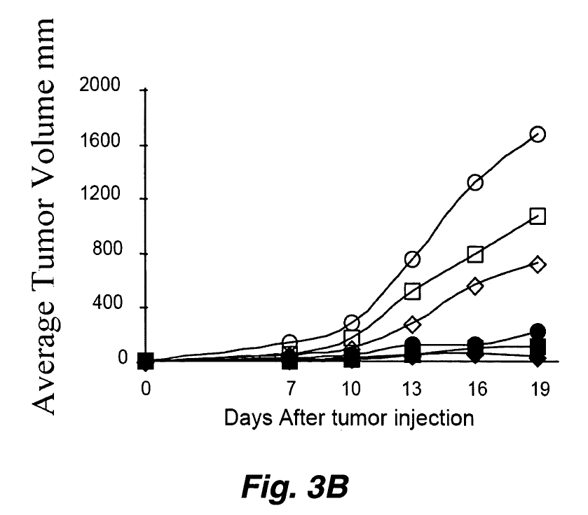

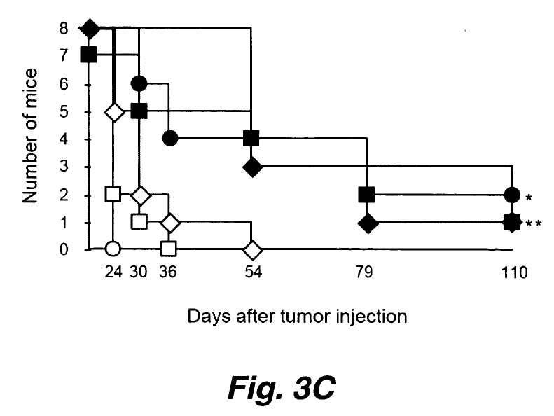

BALB/c mice were vaccinated subcutaneously on week 0 and week 5 with either PBS, ECD alone, ECD^{HER2} plus IgG3 or ECD^{HER2} plus either IgG3-(GMCSF), IgG3-(IL-2) or IgG3-(IL-12) (as described above). No apparent side effects were observed throughout the duration of the vaccination. Eight weeks after the initial vaccination, 10⁶ TUBO cells were injected subcutaneously into the left flank of vaccinated mice. At 7 days post-challenge, measurable tumors were present in all mice vaccinated with PBS, ECD^{HER2} alone and ECD^{HER2} plus IgG3, while two out of eight mice in the ECD^{HER2} plus IgG3-(GMCSF) or ECD^{HER2} plus IgG3-(IL-12) group of vaccinated mice and five out of seven mice in the ECD^{HER2} plus IgG3-(IL-2) vaccinated mice showed no tumors (see, FIG. 3A). Tumors grew uniformly and progressively in all PBS treated mice whereas mice vaccinated with ECD^{HER2} alone and ECD^{HER2} plus IgG3 showed dispersions in the sizes of tumors. The tumors in mice vaccinated with ECD^{HER2} plus antibody-immunostimulant fusion proteins remained smaller or absent in those days indicated (see FIG. 3A).

FIG. 3 displays tumor growth in vaccinated mice challenged with TUBO. As described above, groups of eight female BALB/c mice were vaccinated subcutaneously on day −56 and again on day −21 with either PBS (see, ◯ in FIG. 3), ECD^{HER2} protein alone (see, □ in FIG. 3), ECD^{HER2} plus IgG3 (see, ⋄ in FIG. 3), ECD^{HER2} plus IgG3-(GMCSF) (see, ♦ in FIG. 3), ECD^{HER2} plus IgG3-(IL-2) (see, ▪ in FIG. 3) or ECD^{HER2} plus IgG3-(IL-12) (see, ● in FIG. 3). Again, as described above, on day 0, 10⁶ TUBO cells were injected subcutaneously in the left flank of the mice. The average tumor size of either individual (FIG. 3A) or average (FIG. 3B) were measured starting on day 7 and every three days until day 19. FIG. 3C shows a survival curve of the mice. Mice with tumors exceeding 1.5 cm in diameter at the time of inspection were euthanized and considered to have not survived the challenge. Mice free of tumors at day 110 are indicated by (*).

At day 19, smaller tumors (p<0.02) were apparent in ECD^{HER2} alone and ECD^{HER2} plus IgG3 vaccinated mice as compared to the PBS control. See, FIG. 3B. Tumor size was significantly smaller in those mice vaccinated with ECD^{HER2} plus IgG3-(GMCSF) or mice vaccinated with ECD^{HER2} plus IgG3-(IL-2) in all the days indicated (p≦0.05 as compared to the PBS, ECD^{HER2} alone or ECD^{HER2} plus IgG3 vaccinated mice). See, FIG. 3B. While the average size of tumors of mice vaccinated with ECD^{HER2} plus IgG3-(IL-12) was smaller than those mice vaccinated with PBS, ECD^{HER2} alone, or ECD^{HER2} plus IgG3, significantly smaller tumors were present only on days 13, 16 and 19 (p<0.05) (see, FIG. 3B).

Again, mice bearing tumors greater than 1.5 cm in diameter at the time of inspection were euthanized and considered to have not survived the challenge with TUBO. A survival curve, taking this into consideration, shows the superiority of vaccination regimens in which ECD^{HER2} is combined with antibody-immunostimulant fusion proteins (p<0.05, compared to ECD^{HER2} alone or ECD^{HER2} plus IgG3 vaccinated mice). See, FIG. 3C. No significant difference was observed between the ECD^{HER2} plus IgG3 and the mice vaccinated with ECD^{HER2} alone (p=0.20). At 110 days post-challenge, one out of eight mice vaccinated with ECD^{HER2} plus IgG3-(GMCSF) or ECD^{HER2} plus IgG3-(IL-2) and two out of eight mice vaccinated with ECD^{HER2} plus IgG3-(IL-12) showed no tumor development (see, FIG. 3C (indicated by asterisks)).

Susceptibility of SK-BR-3 Cells (But Not TUBO Cells) to Murine Anti-ECD HEM Mediated Tumor Growth Inhibition In Vitro

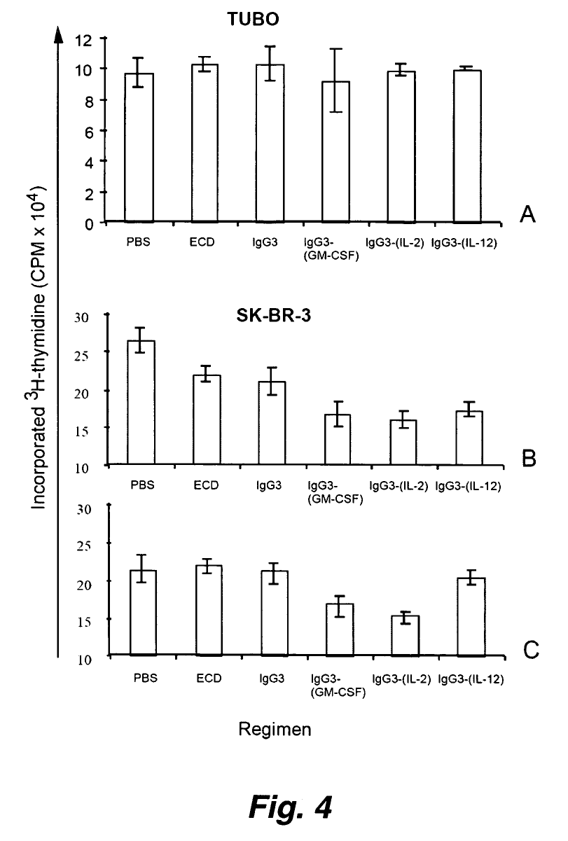

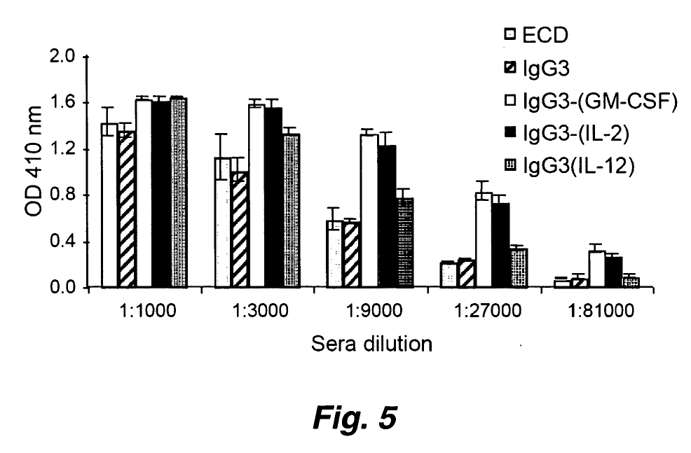

An in vitro proliferation assay was performed to investigate the susceptibility of TUBO cells and SK-BR-3 cells to anti-ECD^{HER2} antibody mediated tumor growth inhibition. No cell growth inhibition was detected when TUBO cells were incubated with the immune sera of vaccinated mice (see, FIG. 4A). With SK-BR-3 cells, immune sera exhibited significant anti-proliferative activity (see, FIG. 4B). As can be seen in FIG. 5, the level of cell growth inhibition correlated with the level of anti-ECD^{HER2} IgG. Immune sera from the mice vaccinated with ECD^{HER2} plus IgG3-(GMCSF) and ECD^{HER2} plus IgG3-(IL-2) exhibited increased growth inhibition, while immune sera of mice vaccinated with ECD^{HER2} plus IgG3-(IL-12) elicited modest inhibition which was still greater than the inhibition in the mice vaccinated with ECD^{HER2} plus IgG3 and ECD^{HER2} alone at the lower sera dilution.

FIG. 4 illustrates the influence of sera on the in vitro proliferation of TUBO cells and SK-BR-3 cells. TUBO or SK-BR-3 cells were incubated with complement-inactivated pooled immune sera obtained from vaccinated mice two days prior to the challenge with TUBO cells. The wells were pulsed with ³H-thymidine 12 hours prior to the end of the incubation. The data in FIG. 4A represent the ³H-thymidine (CPM) incorporated by the TUBO cells after 48 hours of incubation with the immune sera, diluted 1:100, and the data in FIG. 4B represent the level of ³H-thymidine (CPM) incorporated into the SK-BR-3 cells when incubated with immune sera diluted at 1:100 and at 1:300 (FIG. 4C). The error bars represent the range of values obtained.

FIG. 5 illustrates a murine anti-ECD^{HER2} antibody response. Blood samples from vaccinated mice taken two days prior to a challenge with TUBO cells were collected and the sera was pooled. The pooled sera were examined for anti-ECD^{HER2} IgG levels by ELISA. PBS control wells showed undetectable levels of anti-ECD IgG and were used as blanks. Values in FIG. 5 represent the average intensity at OD_{410 nm} of duplicate wells at the indicated serum dilution. The error bars represent the range of duplicate values.

Passive Transfer of Immune Sera

The inability of anti-ECD^{HER2} antibodies to inhibit the growth of TUBO cells in vitro suggested that perhaps an in vivo environment may be necessary to elicit an effective anti-tumor response against the TUBO cells. To examine this possibility, naïve mice were injected intravenously with pooled immune sera and then challenged subcutaneously the next day with 10⁶ TUBO cells. Tumor growth was monitored and measured with a caliper beginning 7 days post-challenge, and every three days following until day 21. Mice injected with sera from mice vaccinated with PBS, ECD^{HER2} alone and ECD^{HER2} plus IgG3 showed no apparent anti-tumor activity throughout the duration of the experiment (see, Table 1, below). As compared to the untreated mice, smaller average size of tumors was observed in mice vaccinated with ECD^{HER2} plus antibody-fusion proteins at the days indicated. However, only at day 13 were significantly smaller tumors observed in those mice injected with sera from mice vaccinated with ECD^{HER2} plus IgG3-(GMCSF) (p=0.03, compared to the untreated mice). Mice injected with sera from mice vaccinated with ECD^{HER2} plus IgG3-(IL-2) showed significantly smaller tumors at days 16 and 19 (p=0.03 and p=0.05 compared to the untreated mice, respectively). Mice injected with sera from mice vaccinated with ECD^{HER2} plus IgG3-(IL-12) showed significantly smaller tumors at days 13, 16, 19 and 21 (p≦0.05 as compared to untreated mice) (see Table 1).

TABLE 1
Passive transfer of immunity^{a}.

| | Average Tumor Size (mm³) | | | | | |
|---|---|---|---|---|---|---|
| Groups | Day 7 | Day 10 | Day 13 | Day 16 | Day 19 | Day 21 |
| Control | 51 | 219 | 439 | 771 | 1045 | 1581 |
| PBS | 50 | 184 | 452 | 589 | 983 | 1468 |
| ECD | 93 | 160 | 451 | 595 | 932 | 1681 |
| IgG3 | 54 | 316 | 432 | 699 | 1242 | 1604 |
| IgG3- | 52 | 140 | 233 | 510 | 897 | 1077 |
| (GM-CSF) | | | | | | |
| IgG3-(IL-2) | 39 | 150 | 261 | 381 | 605 | 977 |
| IgG3-(IL-12) | 19 | 135 | 161 | 389 | 489 | 804 |

^{a}Groups of 6 female BALB/c mice were injected i.v. with 175 μl of pooled immune sera. The following day, day 0, 10⁶ TUBO cells were injected s.c. in the right flank. Tumor growth was examined and measured beginning on day 7 and every three days until day 21. Underlined-bold values indicate the average tumor size of mice in each group with p values <0.05 as compared to the average tumor size of untreated mice.

Characterization of Anti-ECD^{HER2} Antibodies of Transferred Immune Sera

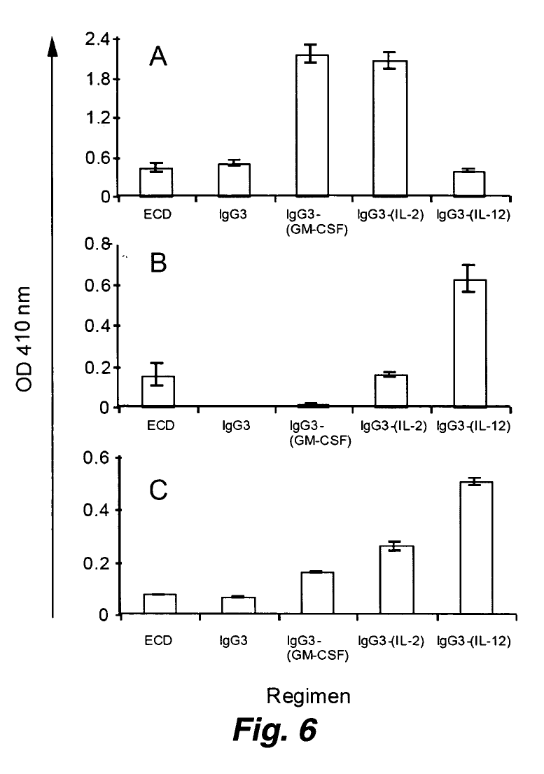

Transferred immune sera were analyzed for the levels of anti-ECD^{HER2} IgG1, IgG2a and IgG3. Pooled serum of mice vaccinated with ECD^{HER2} plus IgG3-(GMCSF) and ECD^{HER2} plus IgG3-(IL-2) showed higher levels of anti-ECD^{HER2} IgG1 as compared to pooled serum of mice vaccinated with ECD^{HER2} plus IgG3-(IL-12), ECD^{HER2} plus IgG3 or ECD^{HER2} alone (see, FIG. 6A). In contrast, the anti-ECD^{HER2} IgG2a response was markedly higher in pooled serum of mice vaccinated with ECD^{HER2} plus IgG3-(IL-12), while a modest response was seen in mice vaccinated with ECD^{HER2} plus IgG3-(IL-2) and lesser levels in ECD^{HER2} plus IgG3-(GMCSF) (see, FIG. 6B). Substantial anti-ECD^{HER2} IgG2a levels were detected in the pooled serum of mice vaccinated with ECD^{HER2} alone comparable to mice vaccinated with ECD^{HER2} plus IgG3-(IL-2), while little to no anti-ECD^{HER2} IgG2a was detected in mice vaccinated with ECD^{HER2} plus IgG3. Analysis of serum of individual mice revealed that one overreacting mouse (out of eight of the mice vaccinated with ECD^{HER2} alone) exhibited a detectable anti-ECD^{HER2} IgG2a response. Little to no detectable anti-ECD^{HER2} IgG2a response was detected in the other seven mice. See, Table 2. Anti-ECD^{HER2} IgG3 levels were similar to anti-ECD^{HER2} IgG2a levels, however, no increased level of anti-ECD^{HER2} IgG3 was observed in pooled serum of mice vaccinated with ECD^{HER2} alone as compared to pooled serum of mice vaccinated with ECD^{HER2} plus IgG3 (see, FIG. 6C).

TABLE 2
Murine anti-ECD^{HER2} IgG2a titers^{a}.

| Mouse | | | | IgG3- | | |
|---|---|---|---|---|---|---|
| No. | PBS | ECD | IgG3 | (GMCSF) | IgG3-(IL-2) | IgG3-(IL-12) |
| 1 | 0^{b} | 0^{b} | 0^{b} | 200 | 200 | 800 |
| 2 | 0^{b} | 8100 | 0^{b} | 100 | 0^{b} | 800 |
| 3 | 0^{b} | 0^{b} | 0^{b} | 200 | 3200 | 800 |
| 4 | 0^{b} | 0^{b} | 0^{b} | 200 | 100 | 6400 |
| 5 | 0^{b} | 0^{b} | 0^{b} | 100 | 100 | 6400 |
| 6 | 0^{b} | 0^{b} | 0^{b} | 100 | 100 | 1600 |
| 7 | 0^{b} | 0^{b} | 0^{b} | 100 | 800 | 800 |
| 8 | 0^{b} | 0^{b} | 0^{b} | 100 | 200 | 800 |
| Average: | 0 | 1013 | 0 | 175 | 586 | 2300 |
| ^{a}Groups of eight female BALB/c mice were injected s.c. in the right flank with either PBS, ECD^{HER2} alone, ECD^{HER2} plus IgG3, ECD^{HER2} plus IgG3-(GM-CSF), ECD^{HER2} plus IgG3-(IL-2), or ECD^{HER2} plus IgG3-(IL-12), at week 0 and again at week 5. At week 8, blood samples were collected and sera from individual mice were examined for anti-ECD^{HER2} IgG2a titers by ELISA. Values represent the average of duplicate | | | | | | |
| # dilutions of serum required to yield an absorbance OD_{410 nm} ≧ 0.05 after 2 hr of incubation. | | | | | | |

As explained above, FIG. 6 illustrates the characterization of anti-ECD^{HER2} IgG of transferred immune sera. Transferred pooled serum from vaccinated mice was examined for anti-ECD^{HER2} IgG1 (see, FIG. 6A), IgG2a (see, FIG. 6B), and IgG3 (see, FIG. 6C) levels by ELISA. Values represent the average intensity at OD_{410 nm} of duplicate wells at 1:1000, 1:50, and 1:50 serum dilution, respectively. PBS control wells were used as blanks. The error bars represent the range of duplicate values.

In Vitro Stimulation of Splenocytes from Vaccinated Mice by ECD^{HER2} Protein

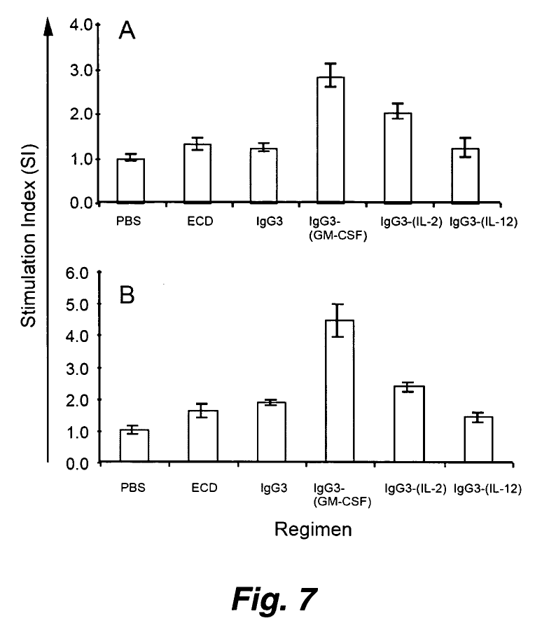

To determine the cellular immune response elicited in vaccinated mice, the ability of splenocytes to proliferate following incubation with soluble ECD^{HER2} protein in vitro was assessed. Proliferation was measured by ³H-thymidine incorporation into DNA. After 48 hours of incubation, significant proliferation was detected in splenocytes from the mice vaccinated with ECD^{HER2} plus IgG3-(GMCSF) with less proliferation seen with splenocytes from mice vaccinated with ECD^{HER2} plus IgG3-(IL-2). Very modest stimulation was observed when the splenocytes were from mice vaccinated with ECD^{HER2} plus IgG3-(IL-12), ECD^{HER2} alone or ECD^{HER2} plus IgG3 (see, FIG. 7A). Similar results were observed after 96 hours of incubation with the soluble ECD^{HER2} protein (see, FIG. 7B).

FIG. 7 displays in vitro stimulation of splenocyte proliferation by ECD^{HER2} protein. Pooled splenocytes from vaccinated mice were incubated with soluble ECD^{HER2} in a 24-well tissue culture plate and pulsed with ³H-thymidine 12 hours prior to the end of the incubation periods (i.e., 48 hours, as seen in FIG. 7A or 96 hours, as seen in FIG. 7B). Cells from a single well of a 24-well tissue culture plate were transferred to a 96-well round bottom tissue culture plate in quadruplicate and collected with a cell harvester. Incorporated ³H-thymidine (CPM) was measured using a scintillation counter. The data in FIG. 7 are expressed as a stimulation index (SI) (as defined above). The error bars represent the range of values obtained from the four determinations.

IFN-γ Production of Stimulated Splenocytes

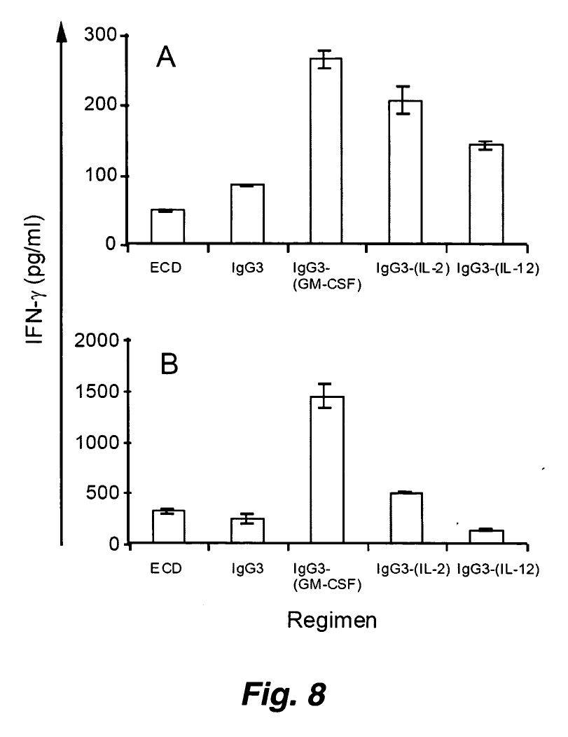

The supernatants of splenocytes incubated with the soluble ECD^{HER2} protein were examined for the levels of the T_{H}1 or T_{H}2 cytokines, IFN-γ and IL-4 (see, e.g., Arai, et al. 1990 “Cytokines: coordinators of immune and inflammatory responses” Annu Rev Biochem 59:783+. After a stimulation period of 36 hours, increased IFN-γ production was detected in the supernatants of splenocytes from vaccinated mice compared to the PBS control with the level as follows: ECD^{HER2} plus IgG3-(GMCSF)>ECD^{HER2} plus IgG3-(IL-2)>ECD^{HER2} plus IgG3-(IL-12)>ECD^{HER2} plus IgG3>ECD^{HER2} alone (sSee, FIG. 8A). No IFN-γ could be detected when splenocytes from mice treated with PBS were used. After 84 hours of stimulation, enhanced IFN-γ production was detected in the supernatant of splenocytes from mice vaccinated with ECD^{HER2} plus IgG3-(GMCSF). The production peaked at approximately 1,500 μg/ml (sSee, FIG. 8B). A modest increase in IFN-γ was observed in the supernatants of mice vaccinated with ECD^{HER2} plus IgG3-(IL-2), while little to no increase was seen in the supernatants of splenocytes from mice vaccinated with ECD^{HER2} plus IgG3-(IL-12), ECD^{HER2} alone or ECD^{HER2} plus IgG3 (see, FIG. 6B). After 36 hours the IL-4 level in all supernatant was below the sensitivity of the assay (<30 μg/ml, data not shown). After 84 hours however, low IL-4 levels could be measured only in the supernatant of splenocytes from mice vaccinated with ECD^{HER2} plus IgG3-(GMCSF) (36 μg/ml).

FIG. 8 displays the in vitro IFN-γ production by stimulated splenocytes from vaccinated mice. Supernatants from splenocytes of vaccinated mice were harvested after either 36 hours (FIG. 8A) or 84 hours (FIG. 8B) of incubation with soluble ECD^{HER2}, and the level of IFN-γ secretion was quantified using a sandwich ELISA. A standard curve was generated in each plate and data was presented as the concentration of IFN-γ (μg/ml) minus the background (PBS control) levels. The error bars represent the range of duplicate values.

### Example II

### Use of Antibody-Immunostimulant Fusion Proteins to Enhance Immune Response Against Staphylococcus aureus Virulence Factor Protein A

Protein A and Antibody-Immunostimulant Fusion Proteins

As outlined above, antibody-immunostimulant (e.g., cytokine) fusion proteins specific for the extracellular domain of the human tumor associated antigen HER2/neu (ECD^{HER2}) were constructed and their action characterized. Such fusion proteins were composed of human IgG3 (containing the variable region of Trastuzumab (Herceptin, Genentech, San Francisco, Calif.)) which was genetically fused to the immunostimulatory cytokines interleukin-2 (IL-2), interleukin-12 (IL-12), or granulocyte-macrophage colony stimulator factor (GMCSF). See, Penichet, M. L. and Morrison, S. L. 2001, “Antibody-cytokine fusion proteins for the therapy of cancer” J Immunol Methods 248: 91-101; Peng, L. S., et al. 1999, “A single-chain IL-12 IgG3 antibody fusion protein retains antibody specificity and IL-12 bioactivity and demonstrates antitumor activity” J Immunol 163: 250-8; and Dela Cruz, J. S., et al. 2000, “Recombinant anti-human HER2/neu IgG3-(GMCSF) fusion protein retains antigen specificity, cytokine function and demonstrates anti-tumor activity” J Immunol 165: 5112-21.

During the work done characterizing anti-HER2/neu fusion proteins (i.e., see above) it was found that antibodies containing the variable regions of Herceptin would bind protein A. This observation was surprising since, by contrast with other isotypes, human IgG3 does not bind protein A. It was shown that such binding occurred through the variable region of the antibodies (Penichet et al., unpublished results). This finding was consistent with a recent report describing the Herceptin variable region as encoded by the V_{H}3 gene family (see, Meininger, D. P., et al. 2000 “Characterization of the binding interface between the E-domain of Staphylococcal protein A and an antibody Fv-fragment” Biochemistry 39: 26-36).

As stated above, antibodies with V_{H}3 regions bind to protein A through the “frame-work” of their variable regions. However, it must be noted that the protein A binding site is separated from the classical Fc binding site (see, Tashiro M., et al. 1995, “Structures of bacterial immunoglobulin-binding domains and their complexes with immunoglobulins” Curr Opin Struct Biol 4: 471-81; Vidal M. A., et al. 1985 “Alternative mechanism of protein A-immunoglobulin interaction the V_{H}-associated reactivity of a monoclonal human IgM” J Immunol 135: 1232-8; Graille M., et al. 2000, “Crystal structure of a Staphylococcus aureus protein A domain complexed with the Fab fragment of a human IgM antibody: structural basis for recognition of B-cell receptors and superantigen activity” Proc Natl Acad Sci USA 97: 5399-404). Thus, the anti-HER2/neu fusion proteins developed (e.g., those illustrated in Example I) also act as a family of “anti-protein A” fusion proteins.

The ability to bind soluble protein A through use of the two of the antibody fusion proteins (i.e., anti-HER2/neu IgG3-(IL-2) and anti-HER2/neu IgG3-(GMCSF) as utilized in Example I) was used to explore if such antibody-immunostimulant fusion proteins where able to enhance the immunogenicity of the protein A bacterial antigen.

Materials and Methods

Mice

Female BALB/c mice (6-8 weeks old) were purchased from Taconic Farms (Germantown, N.Y.). A group of mice were subcutaneously injected with soluble protein A either with or without the presence of antibody or antibody-immunostimulant fusion proteins. An additional group of mice were injected with PBS alone as a control. Each group contained a total of 8 mice per group.

Vaccination with Soluble Protein A

5 μg of soluble protein A (P4931, Sigma. St. Louis, Mo.) was incubated in 1×PBS (phosphate buffered saline) overnight at 4° C. with either 20 μg of anti-HER2/neu IgG3 (the antibody alone, without a fused immunostimulant), anti-HER2/neu IgG3-(GMCSF) or anti-HER2/neu IgG3-(IL-2) at an antibody molar ratio equivalent to 20 μg of IgG3. The following day samples of the mixtures were injected subcutaneously in the right flanks of the mice. A booster was given to each mouse during week 5 in the same flank.

Preparation of Serum for Enzyme Linked ImmunoSorbant Assay (ELISA) Assays

After immunization, the mice were bled every week for 8 weeks. The blood was collected and stored at 4° C. overnight. The following day the serum was collected and stored at −20° C. The sera of all of the mice in each group were pooled and diluted 1:150 to be used for serological studies.

Serological Studies Using ELISA

An ELISA was used to examine the level of any antibody response to soluble protein A and Cowan I (a standard strain of Staphylococcus aureus which expresses the insoluble surface protein, protein A) that was generated in the mice.

A solution of 10% m/v formalin-killed Cowan I (P7155, Sigma) was diluted in carbonate buffer (at approximately a 1:235 dilution) to give an OD₆₅₀ₙₘ of 0.1. This was added to a 96 well plate (Immulon-2, Dyntex Technologies, Chantilly, Va.) at 50 μl per well and incubated overnight at 4° C. Protein A (P3838, Sigma. St. Louis, Mo.) was diluted in carbonate buffer to give a final concentration of 1 μg/ml. The diluted protein A was added to the 96 well plate at 50 μl per well, and incubated overnight at 4° C. A solution of 3% rabbit serum in PBS was used as a diluent and as a blocking solution to prevent the binding of murine antibodies to protein A through the Fc or Fab regions of the antibodies. It has previously been found that an incubation with 3% rabbit serum in PBS is extremely efficient in blocking the binding of murine antibodies to protein A through Fc or Fab regions (Penichet et al. unpublished results). Because of the blocking, the antibody binding detected in the current example is specific for different epitopes of protein A. The collected mouse serum was diluted 1:450 in 1% BSA (bovine serum albumin) in PBS and added to each well at a volume of 50 μl, followed by 1:2 serial dilutions. Pooled anti-sera showing a high antibody titer to insoluble protein A immobilized on Cowan I and to soluble protein A was diluted 1:1350 in 1% BSA in PBS and used as a positive control. An alkaline phosphatase (AP) labeled goat anti-murine IgG diluted 1:20,000 was used to detect bound murine IgG. The 96-well plates were washed 4 times with PBS and AP-substrate (dissolved in diethanolamine buffer) was added to the plates at a volume of 50 μl per well. The sera of mice vaccinated with PBS alone were included as a control and used as a blank to measure the absorbance at 410 nm.

Results

Detection of Antibody Immune Response Using Plates Coated with Soluble Protein A

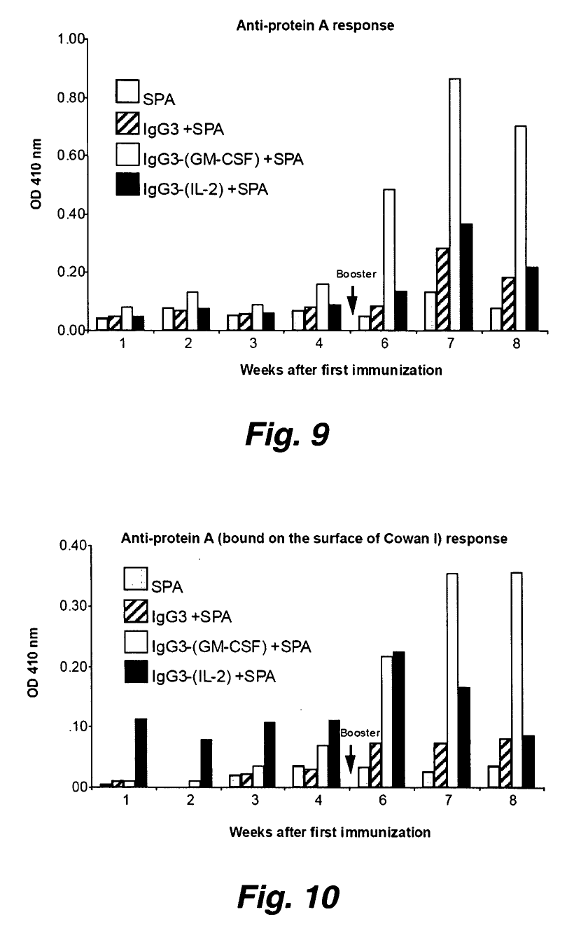

As illustrated in FIG. 9, sera from mice vaccinated with soluble protein A (SPA) in PBS or with IgG3, IgG3-(GMCSF) and IgG3-(IL-2) were collected and pooled weekly for 8 weeks after immunization. The samples were assayed (in duplicate) for anti-protein A response using ELISA with plates coated with soluble protein A. A negative control group consisting of mice given PBS alone, was included and used as a blank. The data in FIG. 9 is presented as the average OD₄₁₀ of duplicate wells. The booster (given 5 weeks after the first vaccination) is indicated by the arrow.

As can be seen in FIG. 9, both the IgG3 and the IgG3-(IL-2) groups did not show an enhanced antibody response to protein A as compared to the PBS group before booster. On the other hand, the IgG3-(GMCSF) group showed an enhanced anti-protein A response. After the booster was given, the IgG3, IgG3-(GMCSF), and IgG3-(IL-2) groups were able to generate enhanced anti-protein A response as compared with the PBS group. Furthermore, after the booster was given, the IgG3-(GMCSF) group generated the greatest enhancement of anti-protein A response as compared to both IgG3 and IgG3-(IL-2) groups.

Detection of Antibody Immune Response Using Plates Coated with Protein A Expressing Cowan I.

Sera from mice vaccinated with soluble protein A (SPA) in PBS or with IgG3, IgG3-(GMCSF) and IgG3-(IL-2) were collected and pooled weekly for 8 weeks after immunization. Using with plates coated with Cowan I, the samples were ELISA-assayed in duplicate for indication of an anti-protein A response (protein A being bound on the surface of Cowan I). A negative control group consisting of mice given PBS alone, was included and used as a blank. The data in FIG. 10 is presented as the average OD₄₁₀ of duplicate wells. A booster was given 5 weeks after the first vaccination (indicated by arrow). As illustrated in FIG. 10, only the IgG3-(IL-2) group generated an enhanced anti-protein A response (protein A being bound on the surface Cowan I) in the weeks before booster. No dramatic change in the later response was observed in the weeks after the booster was given. After the booster was given, the IgG3, IgG3-(GMCSF) and IgG3-(IL-2) groups showed response as compared with the PBS group. Furthermore the IgG3-(GMCSF) group generated the greatest enhancement of response after the booster, as compared to both IgG3 and IgG3-(IL-2) groups.

### Example III

### Vaccination with Novel Combinations of anti-HER2 Cytokines Fusion Proteins and Soluble Protein Antigen Elicits a Protective Immune Response Against HER2 Expressing Tumors

We have previously demonstrated that anti-HER2/IgG3-(IL-2), (IL-12)-IgG3, or IgG3-(GMCSF) antibody fusion proteins (mono-AbFPs) elicit anti-tumor activity against murine tumors expressing HER2/when used as adjuvants of extracellular domain of HER2/(ECD^{HER2}) protein vaccination. We have now studied the effect of combinations of IL-2 and IL-12 or IL-2 and GM-CSF mono-AbFPs during vaccination with ECD^{HER2}. In addition, we developed two novel anti-HER2/IgG3-cytokine fusion proteins in which IL-2 and IL-12 or IL-12 and GMCSF were fused to the same IgG3 molecule (bi-AbFPs). (IL-12)-IgG3-(IL-2) and (IL-12)-IgG3-(GM-CSF) were properly assembled and retained both cytokine activity and the ability to bind antigen. Vaccination of mice with ECD^{HER2} and a combination of cytokines as either bi-AbFPs or two mono-AbFPs activated both Th1 and Th2 immune responses and resulted in significant protection against challenge with a HER2/expressing tumor. Our results suggest that this approach will be effective in the prevention and/or treatment of HER2/expressing tumors.

Introduction.

The proto-oncogene encodes a 185 kDa transmembrane tyrosine kinase growth factor which is overexpressed in various types of cancers including breast, colon, non-small-cell lung, gastric, and ovarian [1, 2]. Overexpression of HER2 plays a direct role in the pathogenesis and aggressiveness of tumors, and is associated with poor prognosis [1, 3-5]. The elevated level of HER2/in malignancies and its extracellular accessibility make it an attractive tumor associated antigen for antibody (Ab) targeted therapy. In fact, Trastuzumab (Herceptin, Genentech, San Francisco, Calif.), an Ab against the extracellular domain of HER2/(ECD^{HER2}) [6], has exhibited efficacy in the treatment of metastatic breast cancer overexpressing HER2 [7]. However, a positive response is observed only in a subset of patients [7], indicating that new modalities to increase clinical efficacy are still needed.

Cytokines play an important role in eliciting and controlling the immune response. The cytokine interleukin-2 (IL-2) is important for the generation of an effective T and NK cell-mediated immune response against malignancies [8]. IL-2 can activate T cells to proliferate and become cytotoxic [9,10], stimulate cytotoxicity in NK cells and macrophages, and induce the generation of LAK cells [11]. Interleukin-12 (IL-12) plays a key role in inducing Th1-mediated CD4⁺ T-cell differentiation [12], stimulation of interferon-(IFN-) secretion [13], and activation of NK cells [14]. IL-12 also has anti-angiogenic activity that can result in hypoxia and apoptosis in solid tumors [15]. Granulocyte macrophage-colony stimulating factor (GM-CSF) is a pleiotropic cytokine with variety of activities including the activation and maturation of antigen-presenting cells as well as activation of humoral and cellular immune responses [16-18].

In several cases the simultaneous use of different cytokines has proven to be more effective in the treatment of cancer than the use of single cytokines. In a gene therapy setting, co-injection of vectors encoding IL-12 and IL-2 resulted in greater regression of murine mammary adenocarcinoma than did the use of either vector alone [19]. An enhanced T-cell immune response was observed in a murine neuroblastoma model when treatment with an IL-2 antibody fusion protein (AbFP) was combined with a cellular tumor vaccine secreting IL-12 [20]. In addition, systemic IL-12 administration potentiates the anti-tumor effect of vaccination with IL-2 producing murine colon carcinoma cells [21] or murine glioma cells [22]. Furthermore, systemic IL-12 combined with pulses of IL-2 results in complete regression of primary and metastatic murine renal carcinoma [23].

The cytokines IL-12 and GM-CSF have potent but distinct anti-tumor properties and complementary immunomodulatory roles, making them good candidates for combined immunotherapy [24]. A transfected murine melanoma cell line expressing both IL-12 and GMCSF was more effective in inducing cytotoxicity and regression of established tumors than was the melanoma cell line expressing either cytokine alone [25]. In addition, tumor immunotherapy with this cytokine combination induced both innate and adaptive anti-tumor immune responses resulting in the eradication of pulmonary murine colon cancer metastases [26]. Moreover, the combined use of GM-CSF and IL-12 for gene therapy induced strong anti-tumor cellular immunity and significant therapeutic efficacy in murine hepatocellular carcinomas compared to the separate use of GM-CSF and IL-12 [27].

In our laboratory we have previously fused anti-human HER2 human IgG3 to human IL-2 [IgG3-(IL-2)] [28], murine IL-12 [(IL-12)-IgG3] [29], or murine GM-CSF [IgG3-(GM-CSF)] [30]. These antibody fusion proteins containing a single cytokine (mono-AbFPs) have proven to be effective as direct anti-tumor agents inhibiting the growth of murine tumors expressing human HER2 [28-30]. As an alternative approach, we have also used IgG3-(IL-2), (IL-12)-IgG3, and IgG3-(GM-CSF) AbFPs as adjuvants of vaccination of mice with ECD^{HER2} protein and have found a significant retardation of the growth of a syngeneic carcinoma expressing rat HER2 in these mice [31].

We have now investigated the immunostimulatory and anti-tumor properties during ECD^{HER2} vaccination of the combination of two cytokines either in the same molecule or on different molecules as a mixture of two mono-AbFPs. We report the construction of two novel anti-HER2 AbFPs consisting of the variable region of the humanized Ab Trastuzumab and the constant region of human IgG3 with murine single chain (msc) IL-12 genetically fused to the amino-terminus, and human IL-2 [(IL-12)-IgG3-(IL-2)], or murine GM-CSF [(IL-12)-IgG3-(GM-CSF)] fused to the carboxy-terminus (bi-AbFPs). These bi-AbFPs bind antigen and retain their cytokine activity. Analysis of the isotypes of anti-ECD^{HER2} antibodies elicited after vaccination with ECD^{HER2} using the AbFPs as adjuvants suggests that the cellular and humoral immune responses are stimulated differently depending on the combination of cytokines used and whether they were administered as bi-AbFPs, or a mixture of mono-AbFPs. Vaccination with bi-AbFPs or a combination of mono-AbFPs resulted in more long-term survivors than did vaccination with ECD^{HER2} alone or with individual mono-AbFPs.

Materials and Methods.

Cell Lines and Culture Conditions.

The murine myeloma cell line P3X63Ag8.653 (American Type Culture Collection [ATCC], Manassas, Va.) and its derivatives expressing recombinant Abs were grown in IMDM (Irvine Scientific, Irvine, Calif.) supplemented with 2 mM L-glutamine, 10 U/ml penicillin, 10 μg/ml streptomycin (GPS) (Sigma Chemical, St. Louis, Mo.) and 5% calf serum (Atlanta Biologicals, Norcross, Ga.). FDC-P1 (ATCC), a GM-CSF-dependent murine myeloid cell line, was cultured in IMDM supplemented with 10% calf serum, 10% WEHI-3-conditioned medium, and GPS. CTLL-2 (ATCC), a clone of murine T cells that requires IL-2 for growth, was cultured in RPMI 1640 (Gibco, Life Technologies, Rockville, Md.), supplemented with 10% calf serum, 50 μM—mercaptoethanol, GPS, and 10 U/ml rhIL-2 (PeproTech, Rocky Hill, N.J.). The TUBO cell line (kindly provided by Dr. Carla De Giovanni, University of Bologna, Italy) derives from a spontaneous mammary lobular carcinoma that occurred in a female BALB-T-transgenic mouse that expresses rat HER2 [32]. BHK/erbB2 cells (kindly provided by Dr. James D. Marks, University of California, San Francisco, Calif.) secretes soluble human ECD^{HER2} [31]. Both, TUBO and BHK/erbB2 cells were cultured in IMDM supplemented with GPS and 10% calf serum.

Construction of Antibody Fusion Proteins

The construction and characterization of anti-HER 2 IgG3, IgG3-(IL-2), IgG3-(GM-CSF), and (IL-12)-IgG3 AbFPs have been previously described [28-30]. They were composed of the heavy and light chain variable regions of the humanized Ab 4D5-8 (rhuMAb HER2, Herceptin, generously provided by Paul Carter, Genentech, San Francisco, Calif.) [6] and the constant region of human IgG3. For the construction of the AbFPs we used human IL-2, murine IL-12, and murine GM-CSF, all of which are active in mouse

To construct the (IL-12)-IgG3-(GM-CSF) heavy chain, the I/I DNA fragment encoding murine GM-CSF fused to the 3′ end of the DNA encoding the C_{H}3 domain of IgG3 [30] was ligated to the I/I DNA fragment encoding mscIL-12 fused to the 5′ end of the DNA encoding the V_{H} region of anti-HER2 IgG3 [29]. Similarly, to construct the (IL-12)-IgG3-(IL-2) heavy chain, the PvuI/NheI DNA fragment encoding mscIL-12 was fused to the PvuI/NheI DNA fragment encoding human IL-2 fused to the 3′ end of the DNA encoding the C_{H}3 domain of IgG3 [28].

Transfection and Expression of the Bi-Functional Antibody-Cytokine Fusion Proteins.

The AbFPs were produced as described elsewhere [34]. The IgG3 heavy chain expression vectors were transfected into P3X63Ag8.653 murine myeloma cell line previously transfected with the expression vector for anti-HER2 human light chain [29,30]. Briefly, 10₇ light chain producing cells were transfected by electroporation with 10 μg of the linearized (IL-12)-IgG3-(IL-2) or (IL-12)-IgG3-(GM-CSF) heavy chain expression vectors. Transfected cells were plated at 2×10₄ cells/well in flat-bottom 96-well tissue culture plates. Stable transfectans were selected with 10 mM histidinol (Sigma) and the best producers identified by ELISA [35]. The size and assembly pattern of (IL-12)-IgG3-(IL-2) and (IL-12)-IgG3-(GM-CSF) were determined by labeling cells overnight in growth medium containing [³⁵S]-methionine (Amersham, Piscataway, N.J.), and immunoprecipitating the secreted protein using rabbit anti-human IgG [36] and Staph A (IgGSorb; The Enzyme Center, Malden, Mass.). Immunoprecipitates were analyzed by SDS-PAGE under reducing and non-reducing conditions [37].

Purification of Antibodies Fusion Proteins and ECD^{HER2}

For the purification of IgG3, (IL-12)-IgG3, IgG3-(GM-CSF), IgG3-(IL-2), (IL-12)-IgG3-(GMCSF), and (IL-12)-IgG3-(IL-2), transfectants were expanded in roller bottles containing IMDM plus 1% Fetalclone serum (HiClone, Logan, Utah) and 1:50 Glutamax (Gibco). Culture supernatants were passed through a protein A column (Sigma) and bound proteins were eluted by low pH, neutralized with Tris-HCl pH 8.0, concentrated, and dialyzed [29]. Soluble human ECD^{HER2} was purified from BHK/erbB2 culture supernatants using affinity chromatography with anti-HER2 IgG3 immobilized on Sepharose 4B (CNBr-activated Sepharose 4B, Amersham Pharmacia Biotech, Upsala, Sweden). The elution, concentration and dialysis procedures were the same as those used for the purification of Abs and AbFPs [31].

Ab Binding to Human HER2/neu Antigen

96-well microtiter plates coated with 1 μg/ml of human ECD^{HER2} were incubated with equivalent molar concentrations of anti-dansyl IgG3 (isotype control), IgG3-(IL-2), IgG3-(GM-CSF), (IL-12)-IgG3, (IL-12)-IgG3-(GM-CSF), or (IL-12)-IgG3-(IL-2), 1:3 serially diluted ranging from 600 ng/ml to 7 ng/ml overnight at 4° C. After washing, bound IgG was detected by incubating with AP-labeled goat anti-human IgG (Zymed, San Francisco, Calif.) for 1 h at 37° C. After washing, -nitrophenyl phosphate disodium dissolved in diethanolamine buffer (Sigma) was added, incubated for 1 h at RT, and the plates read at OD₄₁₀.

Proliferation Assays

IL-12 Proliferation Assay

The bioactivity of IL-12 was determined by a T-cell proliferation assay using human PBMC activated for 3 days in the presence of PHA and IL-2 [29]. PBMC were washed and incubated in 96-well plates for 2 days at 37° C., 5% CO₂ in the presence of equivalent molar concentrations of anti-HER2 IgG3, (IL-12)-IgG3, IgG3-(IL-2), IgG3-(GM-CSF), (IL-12)-IgG3 plus IgG3-(IL-2), (IL-12)-IgG3 plus IgG3-(GM-CSF), (IL-12)-IgG3-(IL-2), or (IL-12)-IgG3-(GM-CSF) serially diluted 1:4 over a range of 3 ng/ml to 10 pg/ml. Proliferation was measured by [3H]-thymidine (ICN, Costa Mesa, Calif.) incorporation after 4 h incubation at 37° C. in 5% CO₂. All measurements were made in quadruplicate.

Assay for GM-CSF Dependent Cell Proliferation

GM-CSF bioactivity was assayed by its ability to support FDC-P1 proliferation [30]. Prior to the experiment, FDC-P1 cells were washed and incubated 12 h in RPMI 1640 supplemented with GPS and 2.5% calf serum at 37° C. in 5% CO₂, in the absence of WEHI-3 conditioned medium. Cells were then incubated in 96-well plates in the presence of serial 1:3 dilutions over a range of 1 ng/ml to 4 pg/ml of equivalent molar concentrations of anti-HER2IgG3, (IL-12)-IgG3, IgG3-(GM-CSF), (IL-12)-IgG3 plus IgG3-(GM-CSF), or (IL-12)-IgG3-(GM-CSF). After 48 h of culture at 37° C. in 5% CO₂, proliferation was measured using the Cell Titer 96 aqueous non-radioactive colorimetric assay (Promega, Madison, Wis.), and plates were read at OD₄₉₀. All measurements were made in quadruplicate.

IL-2 Proliferation Assay

IL-2 bioactivity was determined using the CTLL-2 cell line [38]. Serial 1:3 dilutions ranging from 30 ng/ml to 0.1 ng/ml of equivalent molar concentrations of anti-HER2IgG3, (IL-12)-IgG3, IgG3-(GM-CSF), (IL-12)-IgG3 plus IgG3-(GM-CSF), or (IL-12)-IgG3-(GM-CSF) were added to 96-well plates. CTLL-2 cells were then added to each well and the plates incubated 18 h at 37° C. in 5% CO₂. Proliferation was measured by [³H]-thymidine (ICN, Costa Mesa, Calif.) incorporation during a subsequent 4 h incubation at 37° C. in 5% CO₂. All measurements were made in quadruplicate.

Vaccination of Mice and Challenge with TUBO Cells

AbFPs were mixed with ECD^{HER2} at a 1:2 molar ratio at a concentration to allow the injection of 150 μl per mouse, and incubated at 4° C. overnight. Groups of 8 female BALB/c mice 6 to 8 weeks old (Taconic Farms) were vaccinated with either PBS, 8 μg of ECD^{HER2} alone, or a combination of 8 μg of ECD^{HER2} with 7 μg of IgG3, 8 μg of IgG3-(IL-2), 8 μg of IgG3-(GMCSF), 13 μg of (IL-12)-IgG3, a mixture of 13 μg of (IL-12)-IgG3 and 8 μg of IgG3-(IL-2), a mixture of 13 μg of (IL-12)-IgG3 and 8 μg of IgG3-(GM-CSF), 14 μg of (IL-12)-IgG3-(IL-2), or 14 μg of (IL-12)-IgG3-(GM-CSF). Mice were injected s.c. in the right flank at week 0 and boosted s.c. in the same flank at week 5. Three weeks after the boost, mice were challenged s.c. in the left flank with 10₆ TUBO cells in 150 μl HBSS (Gibco). Tumor growth was monitored with a caliper and the volume calculated using the formula: tumor volume=(length×width²)/2 [39]. Latency was defined as the period of time between the tumor challenge and the detection of a palpable mass of 0.3 cm in diameter, while survival was considered as the period of time from the challenge until when the tumor reached 1.5 cm in diameter and mice were euthanized. The significant differences in latency and survival between experimental groups were analyzed by the non-parametric Peto-Peto-Wilcoxon test.

Determination of Vaccine Immunocomplexes Formation

Aliquots from each vaccination condition, serially diluted 1:10 to 1:640, were added to 96-well plates coated with 1 μg/ml goat anti-human IgG (Zymed) and incubated overnight at 4° C. Following appropriate washes, bound AbFP complexed to ECD^{HER2} was incubated with mouse anti-hu Neu (Santa Cruz Biotechnology, Santa Cruz, Calif.) specific for ECD^{HER2} for 1 h at 37° C. After washing, binding of mouse anti-hu Neu was detected with AP-rabbit anti-mouse IgG (Zymed). After washing, the plates were developed as described previously for AP-conjugates in ELISA.

Assessment of Anti-(ECD^{HER2}) Antibody in Serum

Sera obtained from mice 2 days prior to the challenge with TUBO were analyzed by ELISA for the presence of IgG1 and IgG2a anti-(ECD^{HER2}). 96-well plates coated with 1 μg/ml ECD^{HER2} were incubated overnight at 4° C. with serial dilutions of sera from each experimental group. Bound anti-(ECD^{HER2}) IgG1 or IgG2a was detected by incubating with AP-labeled rat anti-mouse IgG1 or IgG2a respectively (Zymed, San Francisco, Calif.) for 1 h at 37° C. After washing, -nitrophenyl phosphate disodium dissolved in diethanolamine buffer (Sigma) was added, the plates incubated for 1 h at RT and read at OD₄₁₀. Sera from naïve mice of the same age were used as a negative control. All ELISAs were made in duplicate using an internal positive control curve for each plate. Significance in the differences in titers between the different experimental conditions were calculated using the Mann-Whitney test.

Results.

Construction, Expression, and Initial Characterization of (IL-12)-IgG3-(GM-CSF) and (IL-12)-IgG3-(IL-2)

The construction, expression, and characterization of the anti-HER2AbFPs (IL-12)-IgG3, IgG3-(IL-2), and IgG3-(GM-CSF) have been described previously [28-30]. (IL-12)-IgG3 has murine IL-12 subunits p40 and p35 joined by a flexible (Gly₄Ser)₃ linker (p40.linker.p35) genetically fused at the amino-terminus of the V_{H} of an anti-HER2 IgG3 heavy chain through another identical flexible linker. The resulting mono-AbFP retains cytokine activity and the ability to bind antigen [29]. In IgG3-(IL-2) and IgG3-(GM-CSF) the cytokines were fused to the carboxy-terminus of anti-HER2IgG3 [28,30]. We used human IL-2, because this cytokine is active in human and mouse [28]. In contrast, since human IL-12 and human GM-CSF are not active in the mouse, we used murine IL-12 and GM-CSF so that the activity of the AbFPs could be examined in mice [16,33]. Using these AbFPs as a starting point we constructed two novel fusion proteins combining anti-HER2(IL-12)-IgG3 with IgG3-(IL-2) or IgG3-(GMCSF) to create fusion proteins with two cytokines. FIG. 11A shows a diagram of the resulting proteins.

The purified AbFPs were analyzed by SDS-PAGE in the absence (FIG. 11B top panel) or presence (FIG. 11B bottom panel) of the reducing agent—mercaptoethanol. In the absence of reducing agent all of the AbFPs migrate with the apparent m.w. expected for fully assembled H₂L₂ molecules. Hence, the presence of murine IL-12 at the amino-terminus and murine GMCSF or human IL-2 at the carboxy-terminus of anti-HER2 IgG3 does not interfere with the assembly and secretion of the AbFPs. Following treatment with the reducing agent, all proteins yield a band of ˜25 kDa corresponding to the light chain (data not shown) and a higher m.w. band corresponding to the heavy chain at the positions expected for the heavy chain of human IgG3 (60 kDa) [40] fused to mscIL-12 (˜70 kDa) [41], IL-2 (˜16 kDa) [42] or GM-CSF (˜23 kDa) [16] either alone or in combination (FIG. 11B bottom panel).

Antigen Binding Activity

The ability of (IL-12)-IgG3-(IL-2) and (IL-12)-IgG3-(GM-CSF) (FIG. 11C) to bind ECD^{HER2} was examined by ELISA. Both anti-HER2[bi-AbFP as well as their mono-AbFP controls retained the capacity to bind to ECD^{HER2} on coated plates. As expected, anti-dansyl IgG3 did not bind to ECD^{HER2}.

In Vitro Cytokine Activity

The IL-12 dependent proliferation of PHA activated human PBMCs in the presence of both bi-AbFPs (IL-12)-IgG3-(IL-2) (FIG. 12A) and (IL-12)-IgG3-(GM-CSF) (FIG. 12B) was similar to that observed using (IL-12)-IgG3 alone or mixed with IgG3-(IL-2) (FIG. 12A) or IgG3-(GMCSF) (FIG. 12B). IgG3, IgG3-(IL-2), and IgG3-(GM-CSF) did not show any activity in this assay. We used human PBMC because, although human IL-12 is not active in mice, murine IL-12 is functional in both human and mouse [29]. The absence of proliferation in the presence of IL-2 can be explained by the fact that the highest concentration used was below the IL-2 concentration required to stimulate proliferation of PBMCs under the conditions assayed [43]. The lack of proliferation observed in the presence of GM-CSF can be explained by the fact that murine GM-CSF is not functional in human cells [16]. A similar proliferative response by the IL-2 dependent CTLL-2 cell line was observed in response to (IL-12)-IgG3-(IL-2), IgG3-(IL-2) or a mixture of (IL-12)-IgG3 and IgG3-(IL-2) (FIG. 12C). As expected, IgG3 and (IL-12)-IgG3 did not induce proliferation of CTLL-2. (IL-12)-IgG3-(GM-CSF), IgG3-(GM-CSF), and IgG3-(GM-CSF) mixed with (IL-12)-IgG3 were similar in their ability to induce proliferation of the GM-CSF dependent murine cell line FDC-P1 (FIG. 12D). As expected, IgG3 and (IL-12)-IgG3 had no activity. Therefore, the two cytokines fused to both ends of the heavy chain of IgG3 retained activity.

Isotype of the Anti-(ECD^{HER2}) Response in Mice Vaccinated with ECD^{HER2} and Ab-Cytokine Fusion Proteins

The IgG1 and IgG2a antibody responses to ECD^{HER2} were determined in mice vaccinated with the fusion proteins (FIGS. 13A and B). (IL-12)-IgG3-(IL-2) was significantly better than the combination of the two mono-AbFPs (IL-12)-IgG3 and IgG3-(IL-2) in triggering an IgG1 response. In fact, the mixture of (IL-12)-IgG3 and IgG3-(IL-2) resembled (IL-12)-IgG3, eliciting only a poor IgG1 anti-(ECD^{HER2}) response. However, (IL-12)-IgG3-(IL-2) was not different at the <0.05 level from IgG3-(IL-2). Analysis of the IgG2a anti-(ECD HER2) response showed that (IL-12)-IgG3-(IL-2) was similar to (IL-12)-IgG3 and IgG3-(IL-2) and that both were significantly better than any other treatment.

(IL-12)-IgG3-(GM-CSF) and the mixture of (IL-12)-IgG3 and IgG3-(GM-CSF) were similar in their ability to elicit an anti-(ECD HER2) response of the IgG1 isotype and both were superior to (IL-12)-IgG3. However, the IgG1 response triggered by (IL-12)-IgG3-(GM-CSF) and the combination (IL-12)-IgG3 and IgG3-(GM-CSF) was not significantly different from the IgG1 titers triggered by IgG3-(GM-CSF) alone. (IL-12)-IgG3-(GM-CSF) and the mixture of (IL-12)-IgG3 and IgG3-(GM-CSF) were similar in their ability to trigger an anti-(ECD HER2) response of the IgG2a isotype; the bi-AbFP was better than (IL-12)-IgG3 and both were much better than IgG3-(GM-CSF).

Anti-Tumor Effect of Vaccination with ECD^{HER2} and Ab-Cytokine Fusion Proteins

Previous work in our laboratory had demonstrated that enhanced protection against tumor challenge was elicited by the combined use of ECD^{HER2} and the mono-AbFPs (IL-12)-IgG3, IgG3-(IL-2), or IgG3-(GM-CSF) in a prophylactic vaccination setting [31]. We now compared the protection elicited by vaccination with ECD^{HER2} mixed with IL-12 plus IL-2 or IL-12 plus GM-CSF either as bi-AbFPs or as a mixture of two mono-AbFPs, to that elicited by vaccination with ECD^{HER2} and the mono-AbFPs. Sandwich ELISA experiments showed that stable immunocomplexes consisting of ECD^{HER2} bound to AbFPs were formed before injection (data not shown). Following vaccination, mice were challenged with TUBO, a syngeneic murine breast carcinoma expressing rat HER2. The tumors grew rapidly in the groups vaccinated with PBS, ECD^{HER2} or IgG3, with virtually all animals having visible tumors by day 10 (FIGS. 14 and 15A). In contrast, 10 days after tumor challenge only two of the 24 mice vaccinated with the mono-AbFPs and none of the 32 mice vaccinated with two cytokines either as a bi-AbFP or as a mixture of two mono-AbFPs showed tumors (FIG. 15A). Improved survival was seen in all mice vaccinated with the AbFPs compared to mice vaccinated with no cytokine-AbFP (in all cases 0.001, Table 4). 50 days after TUBO challenge only two of the 24 mice vaccinated with PBS, ECD^{HER2} or IgG3 were still alive. In contrast, 21 of the 24 mice vaccinated with the mono-AbFPs, and all of the 32 mice vaccinated with two cytokines as either bi-AbFPs or as a mixture of two mono-AbFPs remained alive (Table 3, FIG. 15B).

TABLE 4
Latency and survival of vaccrnated mice over a period of 250 days
after TUBO challenge. The statistical evaluation of significant differences in latency and
survival between combinations of cytokines versus their respective controls was detennined
by Peto-Peto-Wilcoxon test.

| | Latency | | | | |
|---|---|---|---|---|---|
| | | p value | | | |
| | Median | | | | (IL-12)-IgG3 + IgG3- |
| | Latency | (IL-12)-IgG3- | (IL-12)-IgG3 + IgG3- | (IL-12)-IgG3- | (GM- |
| Treatment | (days) | (IL-2) | (IL-2) | (GM-CSF) | CSF) |
| PBS | 6 | <0.001 | <0.001 | <0.001 | <0.001 |
| ECD^{HER2} | 6 | <0.001 | <0.001 | <0.001 | <0.001 |
| EDC^{HER2} + IgG3 | 11 | <0.001 | <0.001 | <0.001 | <0.001 |
| ECD^{HER2} + IgG3- | 50 | 0.040 | 0.030 | *N/D | N/D |
| (IL-2) | | | | | |
| ECD^{HER2} + IgG3- | 166 | N/D | N/D | 0.5240 | 0.269 |
| (GM-CSF) | | | | | |
| ECD^{HER2} + (IL-12)- | 145 | 0.700 | 0.490 | 0.188 | 0.109 |
| IgG3 | | | | | |
| ECD^{HER2} + (IL-12)- | 171 | 0.827 | **N/A | N/D | N/D |
| IgG3 + IgG3-(IL-2) | | | | | |
| ECD^{HER2} + (IL-12)- | 250 | N/D | N/D | 0.897 | N/A |
| IgG3 + IgG3-(GM- | | | | | |
| CSF) | | | | | |
| ECD^{HER2} + (IL-12)- | 179 | N/A | 0.827 | N/D | N/D |
| IgG3-(IL-2) | | | | | |
| ECD^{HER2} + (IL-12)- | 250 | N/D | N/D | N/A | 0.897 |
| IgG3-(GM-CSF) | | | | | |
| | Survival | | | | |
| | | p value | | | |
| | Median | | | | (IL-12)-IgG3 + IgG3- |
| | Survival | (IL-12)-IgG3- | (IL-12)-IgG3 + IgG3- | (IL-12)-IgG3- | (GM- |
| Treatment | (days) | (IL-2) | (IL-2) | (GM-CSF) | CSF) |
| PBS | 21 | <0.001 | <0.001 | <0.001 | <0.001 |
| ECD^{HER2} | 21 | <0.001 | <0.001 | <0.001 | <0.001 |
| ECD^{HER2} + IgG3 | 29 | <0.001 | <0.001 | <0.001 | <0.001 |
| ECD^{HER2} + IgG3- | 82 | 0.032 | 0.052 | N/D | N/D |
| (IL-2) | | | | | |
| ECD^{HER2} + IgG3- | 226 | N/D | N/D | 0.580 | 0.362 |
| (GM-CSF) | | | | | |
| ECD^{HER2} + (IL- | 175 | 0.622 | 0.555 | 0.130 | 0.077 |
| 12)-IgG3 | | | | | |
| ECD^{HER2} + (IL- | 194 | 0.748 | N/A | N/D | N/D |
| 12)-IgG3 + IgG3- | | | | | |
| (IL-2) | | | | | |
| ECD^{HER2} + (IL- | 250 | N/D | N/D | 1.00 | N/A |
| 12)-IgG3 + IgG3- | | | | | |
| (GM-CSF) | | | | | |
| ECD^{HER2} + (IL- | 196 | | | N/D | N/D |
| 12)-IgG3-(IL-2) | | | | | |
| ECD^{HER2} + (IL- | 250 | N/D | N/D | N/A | 1.00 |
| 12)-IgG3-(GM- | | | | | |
| CSF) | | | | | |

*(N/D) not done; corresponds to comparison between groups with AbFPs that did not share the same cytokines.
**(N/A) not applicable; comparison between the same groups.

Mice vaccinated with the combination of IL-12 and IL-2 either as a bi-AbFP or as mixture of two mono-AbFPs show significant differences in latency and survival compared to those vaccinated with IgG3-(IL-2) alone (0.053), but not with (IL-12)-IgG3 alone (>0.10) (Table 4). Similarly, mice vaccinated with a combination of IL-12 and GM-CSF as mixture of two mono-AbFPs approached statistical superiority in survival (0.077) over mice treated with (IL-12)-IgG3, but not with IgG3-(GM-CSF) (>0.10) (Table 4). However, vaccination with (IL-12)-IgG3-(GM-CSF) was not different in latency and survival compared to (IL-12)-IgG3 or IgG3-(GM-CSF) alone (>0.10) (Table 4). Importantly, 250 days after challenge only 7 (29%) of 24 mice vaccinated with a single mono-AbFP were alive whereas 19 (59%) of 32 animals vaccinated with two cytokines survived (FIG. 15B). Although the sample size is small, the increased percentage of long term survivors following vaccination with two cytokines suggests that there is an advantage in the combination therapy.

Discussion.

In an effort to improve the efficacy of therapies targeting HER 2 expressing malignancies, we have developed a family of AbFPs composed of human IgG3 specific for human HER2/genetically fused to molecules that enhance the immune response including the cytokines IL-12 [29], GM-C SF [30], and IL-2 [28]. These mono-AbFPs were designed to target immunostimulatory molecules to the microenvironment of HER2/expressing tumors with the goal of inducing an enhanced immune response against the tumor cells. They have shown efficacy both as direct anti-tumor agents in the treatment of human HER2/expressing tumors [28-30] and as adjuvants in ECD^{HER2} protein vaccination [31]. Furthermore, recent results demonstrate that the non-covalent physical linkage between the antigen (ECD^{HER2}) and the AbFPs are required to obtain optimal anti-tumor activity during protein vaccination.

In an attempt to obtain a more effective anti-tumor immune response, we have now combined IL-12 with IL-2 and IL-12 with GM-CSF either by genetically fusing both to the same anti-HER2 IgG3 molecule or by mixing two AbFPs containing a single cytokine. We used human IgG3 because its extended hinge region provides spacing and flexibility [44,45], facilitating simultaneous binding to antigen and to the different cytokine receptors. IgG3 is also the most effective human IgG isotype in complement activation and can bind to both human and murine FcRs [46-48]. The presence of two different cytokines in the same molecule guarantees that both will be present at the same place and time, and requires that only one compound be manufactured and characterized. However, the combined use of different AbFPs allows different amounts and dosing schedules to be used with the single components.

In this Example, we show that both anti-HER2/(IL-12)-IgG3-(IL-2) and (IL-12)-IgG3-(GM-CSF) were correctly expressed and retained both the ability to bind antigen and the bioactivity in their cytokine components. Thus, the combination of two cytokines in a single antibody fusion protein did not result in loss of activity of either component. Recently other groups have described bi-AbFPs consisting of IL-12 and IL-2 [49], GM-CSF and IL4 [49], or IL-12 and TNF-[50]. Gillies. [49] tested different configurations of bi-AbFPs in which murine IL-12 and IL-2 were genetically fused to a humanized anti-EpCAM IgG1: a) in tandem to the carboxy-terminus of the heavy chain; b) scIL-12 fused to the amino-terminus and IL-2 to the carboxy-terminus of the heavy chain; and c) scIL-12 fused to the amino-terminus of the light chain and IL-2 fused to the carboxy-terminus of the heavy chain [49]. Of these different configurations, the one with scIL-12 fused to the amino-terminus and IL-2 to the carboxyterminus of the heavy chain is the most similar to our IL-12/IL-2 bi-AbFP. However, the proteins differ in antigen target (EpCAM HER2/), Ab isotype (IgG1. IgG3), IL-12 subunit sequence order (p35-linker-p40 p40-linker-p35), link between IL-12 and Ab (no flexible linker flexible linker), and IL-2 source (murine. human). The anti-EpCAM IL-12/IL-2 AbFP showed effective anti-tumor activity in a murine model of Lewis lung carcinoma when secreted by tumor cells following gene therapy. It was also effective when injected intratumorally, and to a lesser extent, when injected i.v. [49]. Anti-EpCAM GM-CSF/IL4 was functional but was not tested [49]. A bi-AbFP that targets angiogenic tissue via a scFv specific for the extracellular domain of B fibronectin (L19) genetically fused to murine IL-12 and TNF-in the sequence (p40-linker-p35)-linker-scFv-(TNF-) was functional, but showed modest anti-tumor activity compared to the combined use of the mono-AbFPs [50].

The cytokines produced by CD4⁺ T cells stimulated with antigen influence the isotype of the Ab produced by B cells. Th1 cytokines will shift the isotype to IgG2a, while Th2 cytokines will shift the isotype towards IgG1 [51]. In mice vaccinated with ECD^{HER2} plus the IL-12 AbFP, we observed a significant IgG2a anti-(ECD^{HER2}) response, consistent with the role of IL-12 in inducing Th1 CD4⁺ cells [31]. In contrast, the use of IL-2 or GM-CSF AbFPs favored a more vigorous anti-(ECD^{HER2}) IgG1 response [31]. The use of both bi-AbFPs resulted in high titers of IgG1 and IgG2a anti-HER2 suggesting the simultaneous stimulation of the Th1 and Th2 responses. Similarly, vaccination with ECD^{HER2} and IgG3-(GM-CSF) plus (IL-12)-IgG3 also resulted in high titers of anti-HER2IgG1 and IgG2a. However, vaccination with ECD^{HER2} and IgG3-(IL-2) plus (IL-12)-IgG3 elicited high levels of IgG2a, but a significantly lower IgG1 response compared to treatment with (IL-12)-IgG3-(IL-2) or IgG3-(IL-2), suggesting the response mainly influenced by IL-12 and was Th1-like. The decrease in Th2 response using combination of (IL-12)-IgG3 and IgG3-(IL-2) compared to that of IgG3-(IL-2) alone can be explained by the fact that IL-12 not genetically fused to IL-2 strongly favors the differentiation of the immune response from the Th2 (humoral) to a Th1 (cellular) immune response [52].

Previously we had shown that mice vaccinated with ECD^{HER2} complexed with IgG3-(IL-2), IgG3-(GM-CSF), and (IL-12)-IgG3 were more effectively protected against challenge with TUBO cells than those vaccinated with ECD^{HER2} alone or in combination with anti-HER2/IgG3 [31]. We observed that (IL-12)-IgG3-(IL-2) and (IL-12)-IgG3-(GM-CSF), like the mono-AbFPs, were able to bind to ECD^{HER2}. This property is critical since changes in binding to the soluble form of ECD^{HER2} could affect the formation of the Ab/Ag complex required for optimal immunostimulation. TUBO cells serve as an excellent mouse model of breast cancer, growing progressively in BALB/c mice and giving rise to lobular carcinoma histologically similar to that seen in female BALB-T-transgenic mice [32]. Although rat HER2/differs from mouse in 6% of its amino acid sequence [53] and is highly expressed on the membrane of TUBO cells, it does not elicit a detectable antibody or cellular response in wild type BALB/c mice [54]. In addition, the fact that human HER2/shows more than 90% homology with rat HER2/[55] makes TUBO a meaningful read out system for human HER2/vaccination.

We have shown that the combination of IL-12/IL-2 and IL-12/GM-CSF as either two mono-AbFPs or with both cytokines fused to one Ab were effective in enhancing vaccination with ECD HER2. The fact that the bi-AbFPs showed activity is by itself an important contribution since, as was seen for the (IL-12)-L19-(TNF-) fusion protein [50], fusing two cytokines to an antibody does not necessarily result in significant therapeutic effect. We observed that the combination of IL-12/IL-2 was more effective than IL-2 alone, although it was not statistical different from IL-12 alone. Similarly, the combination of IL-12/GM-CSF was more effective than IL-12 alone, although its efficacy was not statistical different from that of GM-CSF alone. However, the simultaneous use of two cytokines increased the number of long-term survivors suggesting that there is an advantage in the combination therapy, although studies using more animals will be required to determine if this is significant. In the vaccination approach the use of bi-AbFPs did not appear to have any significant therapeutic advantage over the simultaneous use of two mono-AbFPs. However, it may be an advantage that one compound alone can achieve the effect of combination of two different compounds, since reduced development and production costs may result. It will be of interest to determine if the bi-AbFPs are more effective than the combination of mono-AbFP in treating established tumors, which requires co-localization to the site of the tumor.

In summary, these studies have analyzed novel bi-AbFPs that retain both cytokine activity and ability to bind antigen. The combined use of IL-2 and IL-12 or GM-CSF and IL-12, either as bi-AbFPs or as a mixture of two mono-AbFPs, can influence the immune response during ECD^{HER2} vaccination and elicit potent anti-tumor activity in an animal model. The ability to generate an active immune response against human HER2/has several potential clinical applications. Prophylactic vaccination might be helpful in patients who are at high-risk to develop HER2/expressing malignancies. In addition, vaccination would stimulate immunological memory and could result in prevention of relapse after surgery or radiation therapy. Furthermore, the use of IL-12 and IL-2 or IL-12 and GM-CSF as a mixture of mono-AbFPs or as bi-AbFPs may trigger a potent anti-tumor response in patients with high levels of circulating ECD^{HER2}.

### REFERENCES

1 Roskoski, R., Jr. The ErbB/HER receptor protein-tyrosine kinases and cancer. Biochem Biophys Res Commun 2004, 319(1), 1-11.
2 Blume-Jensen, P. & Hunter, T. Oncogenic kinase signalling. Nature 2001, 411(6835), 355-365.
3 Slamon, D. J., Clark, G. M., Wong, S. G., Levin, W. J., Ullrich, A. & McGuire, W. L. Human breast cancer: correlation of relapse and survival with amplification of the HER-2/neu oncogene. Science 1987, 235(4785), 177-182.
4 Hayes, D. F., Yamauchi, H., Broadwater, G., Cirrincione, C. T., Rodrigue, S. P., Berry, D. A. et al. Circulating HER-2/erbB-2/c-neu (HER-2) extracellular domain as a prognostic factor in patients with metastatic breast cancer: Cancer and Leukemia Group B Study 8662. Clin Cancer Res 2001, 7(9), 2703-2711.
5 Stark, A., Hulka, B. S., Joens, S., Novotny, D., Thor, A. D., Wold, L. E. et al. HER-2/neu amplification in benign breast disease and the risk of subsequent breast cancer. J Clin Oncol 2000, 18(2), 267-274.
6 Carter, P., Presta, L., Gorman, C. M., Ridgway, J. B., Henner, D., Wong, W. L. et al. Humanization of an anti-p 185HER2 antibody for human cancer therapy. Proc Natl Acad Sci USA 1992, 89(10), 4285-4289.
7 Vogel, C. L., Cobleigh, M. A., Tripathy, D., Gutheil, J. C., Harris, L. N., Fehrenbacher, L. et al. First-line Herceptin monotherapy in metastatic breast cancer. Oncology 2001, 61 Suppl 2, 37-42.
8 Grimm, E. A. & Owen-Schaub, L. The IL-2 mediated amplification of cellular cytotoxicity. J Cell Biochem 1991, 45(4), 335-339.
9 Yron, I., Wood, T., Jr., Spiess, P. J. & Rosenberg, S. A. In vitro growth of murine T cells. V. The isolation and growth of lymphoid cells infiltrating syngeneic solid tumors. J. Immunol. 1980, 125(1), 238-245.
10 Bich-Thuy, L. T., Dukovich, M., Peffer, N. J., Fauci, A. S., Kehrl, J. H. & Greene, W. C. Direct activation of human resting T cells by IL 2: the role of an IL 2 receptor distinct from the Tac protein. J Immunol 1987, 139(5), 1550-1556.
11 Heaton, K. M., Ju, G., Morris, D. K., Delisio, K., Bailon, P. & Grimm, E. A. Characterization of lymphokine-activated killing by human peripheral blood mononuclear cells stimulated with interleukin 2 (IL-2) analogs specific for the intermediate affinity IL-2 receptor. Cell Immunol 1993, 147(1), 167-179.
12 Tsung, K., Meko, J. B., Peplinski, G. R., Tsung, Y. L. & Norton, J. A. IL-12 induces T helper 1-directed antitumor response. J. Immunol. 1997, 158(7), 3359-3565.
13 Puddu, P., Fantuzzi, L., Borghi, P., Varano, B., Rainaldi, G., Guillemard, E. et al. IL-12 induces IFN-gamma expression and secretion in mouse peritoneal macrophages. J Immunol 1997, 159(7), 3490-3497.
14 Chehimi, J., Starr, S. E., Frank, I., Rengaraju, M., Jackson, S. J., Llanes, C. et al. Natural killer (NK) cell stimulatory factor increases the cytotoxic activity of NK cells from both healthy donors and human immunodeficiency virus-infected patients. J Exp Med 1992, 175(3), 789-796.
15 Gee, M. S., Koch, C. J., Evans, S. M., Jenkins, W. T., Pletcher, C. H., Jr., Moore, J. S. et al. Hypoxia-mediated apoptosis from angiogenesis inhibition underlies tumor control by recombinant interleukin 12. Cancer Res 1999, 59(19), 4882-4889.
16. Ruef, C. & Coleman, D. L. Granulocyte-macrophage colony-stimulating factor: pleiotropic cytokine with potential clinical usefulness. Rev. Infect. Dis. 1990, 12(1), 41-62.
17. Soiffer, R., Lynch, T., Mihm, M., Jung, K., Rhuda, C., Schmollinger, J. C. et al. Vaccination with irradiated autologous melanoma cells engineered to secrete human granulocyte-macrophage colony-stimulating factor generates potent antitumor immunity in patients with metastatic melanoma. Proc. Natl. Acad. Sci. USA 1998, 95(22), 13141-13146.
18. Dranoff, G., Jaffee, E., Lazenby, A., Golumbek, P., Levitsky, H., Brose, K. et al. Vaccination with irradiated tumor cells engineered to secrete murine granulocyte-macrophage colony-stimulating factor stimulates potent, specific, and long-lasting anti-tumor immunity. Proc Natl Acad Sci USA 1993, 90(8), 3539-3543.
19. Addison, C. L., Bramson, J. L., Hitt, M. M., Muller, W. J., Gauldie, J. & Graham, F. L. Intratumoral coinjection of adenoviral vectors expressing IL-2 and IL-12 results in enhanced frequency of regression of injected and untreated distal tumors. Gene Ther 1998, 5(10), 1400-1409.
20. Lode, H. N., Xiang, R., Duncan, S. R., Theofilopoulos, A. N., Gillies, S. D. & Reisfeld, R. A. Tumor-targeted IL-2 amplifies T cell-mediated immune response induced by gene therapy with single-chain IL-12. Proc Natl Acad Sci USA 1999, 96(15), 8591-8596.
21. Vagliani, M., Rodolfo, M., Cavallo, F., Parenza, M., Melani, C., Parnmiani, G. et al. Interleukin 12 potentiates the curative effect of a vaccine based on interleukin 2-transduced tumor cells. Cancer Res 1996, 56(3), 467-470.
22. Kikuchi, T., Joki, T., Saitoh, S., Hata, Y., Abe, T., Kato, N. et al. Anti-tumor activity of interleukin-2-producing tumor cells and recombinant interleukin 12 against mouse glioma cells located in the central nervous system. Int J Cancer 1999, 80(3), 425-430.
23. Wigginton, J. M., Komschlies, K. L., Back, T. C., Franco, J. L., Brunda, M. J. & Wiltrout, R. H. Administration of interleukin 12 with pulse interleukin 2 and the rapid and complete eradication of murine renal carcinoma. J Natl Cancer Inst 1996, 88(1), 38-43.
24. Berzofsky, J. A., Ahlers, J. D. & Belyakov, I. M. Strategies for designing and optimizing new generation vaccines. Nat Rev Immunol 2001, 1(3), 209-219.
25. Aruga, A., Tanigawa, K., Aruga, E., Yu, H. & Chang, A. E. Enhanced adjuvant effect of granulocyte-macrophage colony-stimulating factor plus interleukin-12 compared with either alone in vaccine-induced tumor immunity. Cancer Gene Ther 1999, 6(1), 89-95.
26. Hill, H. C., Conway, T. F., Jr., Sabel, M. S., Jong, Y. S., Mathiowitz, E., Bankert, R. B. et al. Cancer immunotherapy with interleukin 12 and granulocyte-macrophage colony-stimulating factor-encapsulated microspheres: coinduction of innate and adaptive antitumor immunity and cure of disseminated disease. Cancer Res 2002, 62(24), 7254-7263.
27. Wang, Z., Qiu, S. J., Ye, S. L., Tang, Z. Y. & Xiao, X. Combined IL-12 and GM-CSF gene therapy for murine hepatocellular carcinoma. Cancer Gene Ther 2001, 8(10), 75 1-8.
28. Penichet, M. L., Dela Cruz, J. S., Shin, S. U. & Morrison, S. L. A recombinant IgG3-(IL-2) fusion protein for the treatment of human HER2/neu expressing tumors. Hum Antibodies 2001, 10(1), 43-49.
29. Peng, L. S., Penichet, M. L. & Morrison, S. L. A single-chain IL-12 IgG3 antibody fusion protein retains antibody specificity and IL-12 bioactivity and demonstrates antitumor activity. J Immunol 1999, 163(1), 250-258.
30. Dela Cruz, J. S., Trinh, K. R., Morrison, S. L. & Penichet, M. L. Recombinant anti-human HER2/neu IgG3-(GM-CSF) fusion protein retains antigen specificity and cytokine function and demonstrates antitumor activity. J Immunol 2000, 165(9), 5112-5121.
31. Dela Cruz, J. S., Lau, S. Y., Ramirez, E. M., De Giovanni, C., Formi, G., Morrison, S. L. et al. Protein vaccination with the HER2/neu extracellular domain plus anti-HER2/neu antibody-cytokine fusion proteins induces a protective anti-HER2/neu immune response in mice. Vaccine 2003, 21(13-14), 1317-1326.
32. Rovero, S., Amici, A., Carlo, E. D., Bei, R., Nanni, P., Quaglino, E. et al. DNA vaccination against rat her-2/Neu p185 more effectively inhibits carcinogenesis than transplantable carcinomas in transgenic BALB/c mice. J Immunol 2000, 165(9), 5 133-142.
33. Schoenhaut, D. S., Chua, A. O., Wolitzky, A. G., Quinn, P. M., Dwyer, C. M., McComas, W. et al. Cloning and expression of murine IL-12. J Immunol 1992, 148(11), 3433-3440.
34. Helguera, G. & Penichet, M. L. “Antibody-Cytokine Fusion Proteins for the Therapy of Cancer” in: Adoptive Immunotherapy: Methods and Protocols, Humana Press, Totowa, N.J., 2005.347-373.
35. Shin, S. U. & Morrison, S. L. Production and properties of chimeric antibody molecules. Methods Enzymol 1989, 178, 459-476.
36. Harlow, E. & Lane, D. Anti-immunoglobulin antibodies. In: Antibodies: a laboratory manual, Cold Spring Harbor Laboratory, New York, 1989.623-631.
37. Challita-Eid, P. M., Penichet, M. L., Shin, S. U., Poles, T., Mosammaparast, N., Mahmood, K. et al. A B7.1-antibody fusion protein retains antibody specificity and ability to activate via the T cell costimulatory pathway. J Immunol 1998, 160(7), 3419-3426.
38. Harvill, E. T. & Morrison, S. L. An IgG3-IL2 fusion protein activates complement, binds Fc gamma RI, generates LAK activity and shows enhanced binding to the high affinity IL2-R. Immunotechnology 1995, 1, 95-105.
39. Carlsson, G., Ekelund, L., Stigsson, L. & Hafstrom, L. Vascularization and tumour volume estimations of solitary liver tumours in rats. Ann Chir Gynaecol 1983, 72(4), 187-191.
40. Penichet, M. L., Shin, S. U. & Morrison, S. L. Fab fusion proteins: Immunoligands. In (Eds. Chamow, S. M. & Ashkenazi, A.) John Wiley & Son, Inc., New York, 1999.15-52.
41. Lieschke, G. J., Rao, P. K., Gately, M. K. & Mulligan, R. C. Bioactive murine and human interleukin-12 fusion proteins which retain antitumor activity in vivo. Nat. Biotechnol. 1997, 15(1), 35-40.
42. Robb, R. J., Kutny, R. M., Panico, M., Morris, H. R. & Chowdhry, V. Amino acid sequence and post-translational modification of human interleukin 2. Proc Natl Acad Sci USA 1984, 81(20), 6486-6490.
43. Gately, M. K., Warrier, R. R., Honasoge, S., Carvajal, D. M., Faherty, D. A., Connaughton, S. E. et al. Administration of recombinant IL-12 to normal mice enhances cytolytic lymphocyte activity and induces production of IFN-gamma in vivo. Int. Immunol. 1994, 6(1), 157-167.
44. Dangl, J. L., Wensel, T. G., Morrison, S. L., Stryer, L., Herzenberg, L. A. & Oi, V. T. Segmental flexibility and complement fixation of genetically engineered chimeric human, rabbit and mouse antibodies. Embo. J. 1988, 7(7), 1989-1994.
45. Phillips, M. L., Tao, M. H., Morrison, S. L. & Schumaker, V. N. Human/mouse chimeric monoclonal antibodies with human IgG1, IgG2, IgG3 and IgG4 constant domains: electron microscopic and hydrodynamic characterization. Mol Immunol 1994, 3 1(15), 1201-1210.
46. Janeway, C. A., Travers, P., Walport, M. & Schlomchick, M. Immunobiology: the immune system in health and disease, Garland Publishing, New York, 2001. 125-154.
47. Tao, M. H. & Morrison, S. L. Studies of aglycosylated chimeric mouse-human IgG. Role of carbohydrate in the structure and effector functions mediated by the human IgG constant region. J. Immunol. 1989, 143(8), 2595-2601.
48. Roitt, I. M., Brostoff, J. & Male, K. (eds.)., Gower Medical Publishing, London, 1989, 5.7-5.8.
49. Gillies, S. D., Lan, Y., Brunkhorst, B., Wong, W. K., Li, Y. & Lo, K. M. Bi-functional cytokine fusion proteins for gene therapy and antibody-targeted treatment of cancer. Cancer Immunol Immunother 2002, 51(8), 449-460.
50. Halin, C., Gafner, V., Villani, M. E., Borsi, L., Berndt, A., Kosmehl, H. et al. Synergistic therapeutic effects of a tumor targeting antibody fragment, fused to interleukin 12 and to tumor necrosis factor alpha. Cancer Res 2003, 63(12), 3202-3210.
51. Stevens, T. L., Bossie, A., Sanders, V. M., Fernandez-Botran, R., Coffman, R. L., Mosmann, T. R. et al. Regulation of antibody isotype secretion by subsets of antigen-specific helper T cells. Nature 1988, 334(6179), 255-258.
52. Manetti, R., Parronchi, P., Giudizi, M. G., Piccinni, M. P., Maggi, E., Trinchieri, G. et al. Natural killer cell stimulatory factor (interleukin 12 IL-12]) induces T helper type 1 (Th1)-specific immune responses and inhibits the development of IL-4-producing Th cells. J Exp Med 1993, 177(4), 1199-1204.
53. Nagata, Y., Furugen, R., Hiasa, A., Ikeda, H., Ohta, N., Furukawa, K. et al. Peptides derived from a wild-type murine proto-oncogene c-erbB-2/HER2/neu can induce CTL and tumor suppression in syngeneic hosts. J Immunol 1997, 159(3), 1336-1343.
54. Curcio, C., Di Carlo, E., Clynes, R., Smyth, M. J., Boggio, K., Quaglino, E. et al. Nonredundant roles of antibody, cytokines, and perforin in the eradication of established Her-2/neu carcinomas. J Clin Invest 2003, 111(8), 1161-70.
55. Yamamoto, T., Ikawa, S., Akiyama, T., Semba, K., Nomura, N., Miyajima, N. et al. Similarity of protein encoded by the human c-erb-B-2 gene to epidermal growth factor receptor. Nature 1986, 3 19(6050), 230-4.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A composition, comprising a chimeric moiety comprising an antibody attached to a first immunostimulant and to a second immunostimulant, wherein said antibody binds a disease-related antigen and said chimeric moiety comprises an effective adjuvant of said disease-related antigen.

2. The composition of claim 1, wherein said chimeric moiety is a fusion protein.

3. The composition of claim 1, wherein the first immunostimulant and/or the second immunostimulant comprises a cytokine domain, a cytokine sequence, a subsequence of a cytokine, a chemokine domain, a chemokine sequence, a subsequence of a chemokine, or an immunostimulant other than a cytokine or a chemokine.

4. The composition of claim 1, wherein the first immunostimulant and the second immunostimulant are independently selected from the group consisting of: cytokines, chemokines, interleukins, interferons, C-X-C chemokines, C-C family chemokines, C chemokines, CX3C chemokines, super antigens, growth factors, IL-1, IL-2, IL-4, IL-6, IL-7, IL-8, IL-10, IL-12, IL-13, IL-17, IL-18, RANTES, mip1α, mip1β, GMCSF, GCSF, gamma interferon, alpha interferon, TNF, CSFs, mip2α, mip2β, PF4, platelet basic protein, hIP10, LD78, Act-2, MCAF, 1309, TCA3, IP-10, lymphotactin, fractalkine, KLH, and fragments thereof.

5. The composition of claim 2, wherein the antibody-immunostimulant fusion protein comprises a linker.

6. The composition of claim 1, wherein the chimeric moiety comprises an antibody specific for a HER2/neu antigen.

7. The composition of claims 1, 2 or, wherein said first immunostimulant is selected from the group consisting of IL-12, IL2, and GM-CSF.

8. The composition of claim 7, wherein said second immunostimulant is different from said first immunostimulant and is selected from the group consisting of IL-12, IL2, and GM-CSF.

9. The composition of claim 1, wherein the chimeric moiety comprises an antibody specific for an antiben selected from the group consisting of a tumor antigen, a bacterial antigen, a viral antigen, a mycoplasm antigen, a fungal antigen, a prion antigen, an autoimmune disorder antigen, and a parasite antigen.

10. The composition of claim 1, wherein said chimeric moiety comprises an antibody specific for a disease-related antigen other than a tumor antigen.

11. The composition of claim 1, wherein said chimeric moiety comprises an antibody specific for a disease-related antigen other than HER2/neu.

12. The composition of claim 1, wherein said chimeric moiety comprises a domain selected from the group consisting of: an antibody fragment, an Fab domain, an Fab′ domain, an F(ab′)2 domain, an F(ab)2 domain and a single chain antibody.

13. The composition of claim 1, wherein said chimeric moiety comprises a domain selected from the group consisting of: IgG, IgA, IgE, IgM, IgD, IgG1, IgG2, and IgG3.

14. The composition of claim 1, wherein said antigen comprises one more antigens selected from the group consisting of a soluble antigen, a soluble antigen bound to a matrix, an insoluble antigen bound to a matrix, an insoluble aggregate of antigens, a nonviable cell-associated antigen, a nonviable organism-associated antigen, and an antigen conjugated with a liposome.

15. The composition of claim 1, wherein said antigen comprises HER2/neu or HER2/neu shed from a tumor cell, or a fragment thereof.

16. The composition of claim 1, wherein said antigen comprises an antigen other than a tumor antigen.

17. The composition of claim 1, wherein said antigen comprises an antigen selected from the group consisting of an antigen arising from a subject, an antigen arising from a disease state within the subject, an antigen arising from a disease related organism within the subject.

18. The composition of claim 17, wherein said antigen arises from a disease state within the subject caused by an agent selected from the group consisting of a tumor, a bacteria, a virus, a mycoplasm, a fungus, a prion, an autoimmune disorder, and an infectious parasite.

19. The composition of claim 17, wherein the antigen comprises an antigen selected from the group consisting of a tumor antigen, a bacterial antigen, a viral antigen, a mycoplasm antigen, a prion antigen, an autoimmune disorder related antigen, an an infectious parasite antigen.

20. The composition of claim 1, wherein the antigen comprises an exogenous antigen.

21. The composition of claim 20, wherein the exogenous antigen comprises an antigen substantially identical to an antigen arising from a disease state within a subject or from a disease related organism within the subject.

22. The composition of claim 1, further comprising said antigen.

23. The composition of claim 22, wherein a ratio of the number of molecules of chimeric moiety to number of molecules of antigen is approximately 1:1.

24. The composition of claim 22, wherein a ratio of the number of molecules of chimeric moiety is substantially greater than the number of molecules of antigen.

25. The composition of claim 22, wherein a ratio of the number of molecules of chimeric moiety to number of molecules of antigen is such that said chimeric moiety is substantially saturated by the antigen.

26. The composition of claim 22, wherein the antigen and the antibody-immunostimulant fusion protein are incubated together for at least one hour.

27. The composition of claim 1, wherein said composition further comprises a pharmaceutically acceptable excipient.

28. A method of inducing or enhancing an immune response in a mammal, said method comprising administering to said mammal a composition comprising a chimeric moiety comprising an antibody attached to a first immunostimulant and to a second immunostimulant, wherein said antibody binds a disease-related antigen and said chimeric moiety comprises an effective adjuvant of said disease-related antigen, where said composition is administered in an amount sufficient to induce a measurable immune response.

29. The method of claim 28, wherein said mammal is a human.

30. The method of claim 28, wherein said mammal is a human having or at risk for cancer.

31. The method of claim 28, wherein said chimeric moiety is a fusion protein.

32. The method of claim 28, wherein the first immunostimulant and/or the second immunostimulant comprises a cytokine domain, a cytokine sequence, a subsequence of a cytokine, a chemokine domain, a chemokine sequence, a subsequence of a chemokine, or an immunostimulant other than a cytokine or a chemokine.

33. The method of claim 28, wherein the first immunostimulant and the second immunostimulant are independently selected from the group consisting of:
cytokines, chemokines, interleukins, interferons, C-X-C chemokines, C-C family chemokines, C chemokines, CX3C chemokines, super antigens, growth factors, IL-1, IL-2, IL-4, IL-6, IL-7, IL-8, IL-10, IL-12, IL-13, IL-17, IL-18, RANTES, mip1α, mip1β, GMCSF, GCSF, gamma interferon, alpha interferon, TNF, CSFs, mip2α, mip2β, PF4, platelet basic protein, hIP10, LD78, Act-2, MCAF, 1309, TCA3, IP-10, lymphotactin, fractalkine, KLH, and fragments thereof.

34. The method of claim 31, wherein the antibody-immunostimulant fusion protein comprises a linker.

35. The method of claim 28, wherein the chimeric moiety comprises an antibody specific for a HER2/neu antigen.

36. The method of claim 28, 31, or 35, wherein said first immunostimulant is selected from the group consisting of IL-12, IL2, and GM-CSF.

37. The method of claim 36, wherein said second immunostimulant is different from said first immunostimulant and is selected from the group consisting of IL-12, IL2, and GM-CSF.

38. The method of claim 28, wherein the chimeric moiety comprises an antibody specific for an antiben selected from the group consisting of a tumor antigen, a bacterial antigen, a viral antigen, a mycoplasm antigen, a fungal antigen, a prion antigen, an autoimmune disorder antigen, and a parasite antigen.

39. The method of claim 28, wherein said chimeric moiety comprises an antibody specific for a disease-related antigen other than a tumor antigen.

40. The method of claim 28, wherein said chimeric moiety comprises an antibody specific for a disease-related antigen other than HER2/neu.

41. The method of claim 28, wherein said chimeric moiety comprises a domain selected from the group consisting of: an antibody fragment, an Fab domain, an Fab′ domain, an F(ab′)2 domain, an F(ab)2 domain and a single chain antibody.

42. The method of claim 28, wherein said chimeric moiety comprises a domain selected from the group consisting of: IgG, IgA, IgE, IgM, IgD, IgG1, IgG2, and IgG3.

43. The method of claim 28, wherein said antigen comprises one more antigens selected from the group consisting of a soluble antigen, a soluble antigen bound to a matrix, an insoluble antigen bound to a matrix, an insoluble aggregate of antigens, a nonviable cell-associated antigen, a nonviable organism-associated antigen, and an antigen conjugated with a liposome.

44. The method of claim 28, wherein said antigen comprises HER2/neu or HER2/neu shed from a tumor cell, or a fragment thereof.

45. The method of claim 28, wherein said antigen comprises an antigen other than a tumor antigen.

46. The method of claim 28, wherein said antigen comprises an antigen selected from the group consisting of an antigen arising from a subject, an antigen arising from a disease state within the subject, an antigen arising from a disease related organism within the subject.

47. The method of claim 46, wherein said antigen arises from a disease state within the subject caused by an agent selected from the group consisting of a tumor, a bacteria, a virus, a mycoplasm, a fungus, a prion, an autoimmune disorder, and an infectious parasite.

48. The method of claim 46, wherein the antigen comprises an antigen selected from the group consisting of a tumor antigen, a bacterial antigen, a viral antigen, a mycoplasm antigen, a prion antigen, an autoimmune disorder related antigen, an an infectious parasite antigen.

49. The method of claim 28, wherein the antigen comprises an exogenous antigen.

50. The method of claim 49, wherein the exogenous antigen comprises an antigen substantially identical to an antigen arising from a disease state within a subject or from a disease related organism within the subject.

51. The method of claim 28, wherein said composition further comprising said antigen.

52. The method of claim 51, wherein a ratio of the number of molecules of chimeric moiety to number of molecules of antigen is approximately 1:1.

53. The method of claim 51, wherein a ratio of the number of molecules of chimeric moiety is substantially greater than the number of molecules of antigen.

54. The method of claim 51, wherein a ratio of the number of molecules of chimeric moiety to number of molecules of antigen is such that said chimeric moiety is substantially saturated by the antigen.

55. The method of claim 51, wherein the antigen and the antibody-immunostimulant fusion protein are incubated together for at least one hour.

56. The method of claim 28, wherein said composition further comprises a pharmaceutically acceptable excipient.

57. The method of claim 28, wherein the method comprises administering to the subject an effective amount of an antibody-immunostimulant fusion protein, administering a disease related antigen, wherein the fusion protein comprises an effective adjuvant of the disease related antigen

58. A method of prophylactically or therapeutically treating a disease state in a subject, the method comprising: administering to the subject an effective amount of an antibody-immunostimulant chimeric moiety, wherein the chimeric moiety comprises an effective adjuvant of a disease related antigen arising from the subject, arising from a disease state within the subject, or arising from a disease related organism within the subject and wherein such administration elicits an immune response within the subject against the disease related antigen.
